(19) United States
(12) Patent Application Publication
(10) Pub. No.: US 2008/0260824 A1
(43) Pub. Date: Oct. 23, 2008

(54) Bioadhesive Rate-Controlled Oral Dosage Formulations

(75) Inventors: Avinash Nangia, Sharon, MA (US); Jules Jacob, Taunton, MA (US); Peyman Moslemy, Mansfield, MA (US)

Correspondence Address: ROPES & GRAY LLP, PATENT DOCKETING 39/41, ONE INTERNATIONAL PLACE, BOSTON, MA, 02110-2624, US

(73) Assignee: Spherics, Inc., Mansfield, MA (US)

(21) Appl. No.: 11/661,540

(22) PCT Filed: Aug. 29, 2005
(86) PCT No.: PCT/US05/30681

§ 371 Date: Mar. 11, 2008

Related U.S. Application Data
(63) Continuation of application No. 11/009,327, filed on Dec. 9, 2004.
(60) Provisional application No. 60/605,200, filed on Aug. 27, 2004.
(60) Provisional application No. 60/604,990, filed on Aug. 27, 2004.
(60) Provisional application No. 60/605,201, filed on Aug. 27, 2004.
(60) Provisional application No. 60/605,198, filed on Aug. 27, 2004.
(60) Provisional application No. 60/605,199, filed on Aug. 27, 2004.
(60) Provisional application No. 60/604,991, filed on Aug. 27, 2004.
(60) Provisional application No. 60/607,905, filed on Sep. 8, 2004.
(60) Provisional application No. 60/528,042, filed on Dec. 9, 2003.
(60) Provisional application No. 60/605,201, filed on Aug. 27, 2004.
(60) Provisional application No. 60/605,199, filed on Aug. 27, 2004.
(60) Provisional application No. 60/607,905, filed on Sep. 8, 2004.
(60) Provisional application No. 60/604,990, filed on Aug. 27, 2004.
(60) Provisional application No. 60/635,812, filed on Dec. 13, 2004.
(60) Provisional application No. 60/650,191, filed on Feb. 4, 2005.
(60) Provisional application No. 60/650,375, filed on Feb. 4, 2005.
(60) Provisional application No. 60/676,383, filed on Apr. 29, 2005.

(51) Int. Cl. A61K 9/22; A61K 9/00; A61K 31/52; A61K 31/19; A61K 31/4436; A61P 43/00; A61K 31/17; A61K 9/14; A61K 9/32

(52) U.S. Cl. 424/468; 424/400; 514/263.3; 514/561; 514/342; 514/592; 514/557; 424/497; 424/482

(57) ABSTRACT

The present invention relates to a bioadhesive drug delivery system (BIOadhesive Rate controlled Oral Dosage (BIOROD) formulation) in which a drug containing core either alone or coated with a rate controlling membrane system is enveloped on its circumference by a bioadhesive coating, thereby yielding a monolithic system that allows for drug release in a regulated manner. Also described herein are polymers with improved bioadhesive properties and methods for improving bioadhesion of polymers.

### RELATED APPLICATIONS

This application claims the benefit of U.S. Provisional Application Nos. 60/604,990, filed Aug. 27, 2004, 60/604,991, filed Aug. 27, 2004, 60/605,198, filed Aug. 27, 2004, 60/605,199, filed Aug. 27, 2004, 60/605,200, filed Aug. 27, 2004, 60/605,201, filed Aug. 27, 2004, 60/607,905, filed Sep. 8, 2004, 60/635,812, filed Dec. 13, 2004, 60/650,191, filed Feb. 4, 2005, 60/650,375, filed Feb. 4, 2005 and 60/676,383, filed Apr. 29, 2005. This application is also a continuation-in-part of U.S. application Ser. No. 11/009,327, filed Dec. 9, 2004. The entire teachings of the above-referenced applications are incorporated herein by reference.

### BACKGROUND OF THE INVENTION

Controlled release systems for drug delivery are often designed to administer drugs in specific areas of the body. In the case of drug delivery to the gastrointestinal tract, it is important that the drug not be delivered substantially beyond the desired site of action or absorption, respectively, before it has had a chance to exert a topical effect or to pass into the bloodstream. A drug delivery system that adheres to the lining of the appropriate viscus will deliver its contents to the targeted tissue as a function of proximity and duration of contact.

Important to the safety and effectiveness of any pharmaceutical formulation is its ability to maintain a target blood level of the active pharmaceutical agent within the agent's therapeutic concentration range. The window of absorption for certain drugs presents a serious challenge to the development of effective modified-release preparations of these compounds. The poor or decreased absorption of these drugs may be attributed to a variety of barriers, which may be biological or physico-chemical in nature, and can be, but are not limited to poor solubility, low permeability, and saturable active absorption or influx mechanisms such as carrier-mediated transport. Poor solubility over a broad pH range is another barrier that inhibits absorption and overall bioavailability for a number of compounds. Furthermore, when solubility is limited at the higher pH's found in the distal gastrointestinal (GI) tract, a limited window of absorption is effectively created.

Such windows of absorption can significantly curtail the bioavailability of a compound and the extent to which Tₘₐₓ, the time at which the rate of absorption of an active agent into the bloodstream is equal to its rate of elimination from the bloodstream, can be extended using conventional modified release dosage forms known in the art.

For example, there is a need for developing gastroretentive bioadhesive formulations for blood glucose lowering drugs that are suitable for administration to patients suffering from type-2 diabetes, also known as non-insulin-dependent diabetes mellitus. For example, the absorption of the anti-diabetic agent, metformin, in humans is incomplete and the drug is excreted mainly in urine with a half life of 4 to 6 h. Metformin is protonated under physiological pH conditions. Ionized metformin is absorbed to the negatively charged intestinal epithelium. The absorption window is predominantly in the small intestine, and colonic absorption in healthy human subjects is poor (Marathe et al., Br J Clin Pharmacol 2000; 50: 325-332). A conventional oral sustained-release formulation releases the drug throughout the small intestine and the colon. However, the drug release after the small intestine would be of no therapeutic value and the conventional strategy of prolonging the metformin release from the dosage form throughout the gastrointestinal (GI) tract will not be effective. Evaluation of five commercial brands of Metformin HCl sustained release tablets, each containing 500 mg of Metformin, revealed no gastroretentive or bioadhesion tendency of these brands (Patel et al., Drug Delivery Technology 2005; 5:38-46). The lack of gastroretentive tendency of a controlled release oral formulation would result in the displacement of the dosage form from the site of absorption and erratic absorption as the drug passes to the colon. Metformin therapy with immediate-release or modified-release formulations, on the other hand, is associated with a high incidence of side effects such as diarrhea, nausea, vomiting, flatulence, etc.

Another drug used in the treatment of type 2 diabetes is glipizide. Gastrointestinal absorption of glipizide is uniform, rapid, and essentially complete. It has a short half-life (2-4 h) with no plasma accumulation upon repeated oral administration. Due to the short elimination half-life, an effective glipizide therapy requires twice daily dosing in a large number of patients (Berelowitz et al., Diabetes Care 1994; 17:1460-1464; Foster and Plosker, Pharmacoeconomics 2000; 18:289-306), which often leads to non-compliance.

It would therefore be useful to have dosage formulations that can deliver, for example, one or more anti-diabetic agents, such as metformin, glipizide, and/or other anti-diabetic agents, such as rosiglitazone and/or pioglitazone at a controlled rate in a substantially constant dose per unit time in the stomach or duodenum for its beneficial therapeutic effects and for better patient compliance. Moreover, there is a need for delivering more than one drug in a single dosage formulation that enhances the retention time of the drugs at a target absorption site within the GI tract.

An orally ingested product can adhere to either the epithelial surface or the mucus lining of the gastrointestinal tract. For the delivery of bioactive substances, it can be advantageous to have a polymeric drug delivery device adhere to the epithelium or to the mucous layer. Bioadhesion in the gastrointestinal tract proceeds in two stages: (1) viscoelastic deformation at the point of contact of the synthetic material into the mucus substrate, and (2) formation of bonds between the adhesive synthetic material and the mucus or the epithelial cells. In general, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., Van der Waals interactions) and stronger specific interactions, which include hydrogen bonds. The hydrophilic functional groups primarily responsible for forming hydrogen bonds are the hydroxyl and the carboxylic acid groups.

Duchene et al., in Drug Dev. EId. Pharm., 14:283-318 (1988), review the pharmaceutical and medical aspects of bioadhesive systems for drug delivery. Polycarbophils and acrylic acid polymers were noted as having the best adhesive properties. “Bioadhesion” is defined as the ability of a material to adhere to a biological surface for an extended period of time. Bioadhesion is one solution to the problem of inadequate residence time resulting from the stomach emptying and intestinal peristalsis, and from displacement by ciliary movement. Bioadhesion results from a confluence of factors, including an intimate contact between the bioadhesive and the receptor tissue, penetration of the bioadhesive into the crevice of the tissue surface and/or mucus, and formation of mechanical, electrostatic, or chemical bonds. Bioadhesive properties of polymers are affected by both the nature of the polymer and by the nature of the surrounding media.

Others have explored the use of bioadhesive polymers; however, the extent of bioadhesion achieved in these studies has been limited. In addition, these studies do not demonstrate how to prepare larger bioadhesive drug delivery devices, such as tablets. WO 93/21906 discloses methods for fabricating bioadhesive microspheres and for measuring bioadhesive forces between microspheres and selected segments of the gastrointestinal tract in vitro. Smart et al., J. Pharm. Pharmacol., 36:295-299 (1984), report a method to test adhesion to mucosa using a polymer-coated glass plate contacting a dish of mucosa. A variety of polymeric materials were tested, including sodium alginate, sodium carboxymethyl-cellulose, gelatin, pectin and polyvinylpyrrolidone. Gurney et al., Biomaterials, 5:336-340 (1984) report that adhesion may be affected by physical or mechanical bonds; secondary chemical bonds; and/or primary, ionic or covalent bonds. Park et al., “Alternative Approaches to Oral Controlled Drug Delivery: Bioadhesives and In-Situ Systems,” in J. M. Anderson and S. W. Kim, Eds., “Recent Advances in Drug Delivery,” Plenum Press, New York, 1984, pp. 163-183, report a study of the use of fluorescent probes in cells to determine adhesiveness of polymers to mucin/epithelial surface, which indicated that anionic polymers with high charge density appear to be preferred as adhesive polymers. Mikos et al., in J. Colloid Interface Sci., 143:366-373 (1991) and Lehr et al., J. Controlled Rel. Soc., 13:51-62 (1990) report a study of the bioadhesive properties of polyanhydrides and polyacrylic acid, respectively, in drug delivery. Lehr et al. screened microparticles formed of copolymers of acrylic acid using an in vitro system and determined that the copolymer “Polycarbophil” has increased adhesion.

Although bioadhesive-coated microparticles have been described, larger dosage forms such as tablets with the ability to adequately adhere to the gastrointestinal tract mucosa would be advantageous. Larger oral formulations cannot enter into an invagination in the mucosa, whereas microparticles are generally small enough to fit into an invagination. As a result, larger oral formulations contact a smaller surface area of the gastrointestinal tract particularly as a function of the ratio of contact surface area to volume of the formulation), which would be expected to weaken the interaction between the larger formulation and the gastrointestinal tract and subject the formulation to increased sheer.

Separately or in addition to the need to control the location at which a drug is released, there is also a need to control the duration over which a drug is released from a pharmaceutical formulation. In particular, certain drugs, especially neuroactive drugs, have side effects and lower efficacy if blood serum concentrations vary considerably. Standard immediate release formulations typically cause such fluctuations in blood serum concentrations, because they dump large quantities of drug at one time into the patient's gastrointestinal tract.

Thus, there is a need for methods for controlling or increasing the absorption of pharmaceutical agents from drug delivery systems such as tablets through mucosal membranes. There also is a need for methods for delaying transit of pharmaceutical formulations through gastrointestinal passages.

### BRIEF SUMMARY OF THE INVENTION

The present invention provides a bioadhesive drug delivery system (BIOadhesive Rate-controlled Oral Dosage (BIOROD) formulation) in which a bioadhesive coating is disposed over all or a portion of the surface of a core containing an agent, which core may optionally be coated with a rate-controlling membrane system, thus yielding a monolithic system that releases the agent in a regulated manner. Polymers with improved bioadhesive properties and methods for improving bioadhesion of polymers have been developed. For example, a compound containing an aromatic group which bears one or more hydroxyl groups may be grafted onto a polymer or coupled to individual monomers. The monomers may then be polymerized to form any type of polymer, including biodegradable and non-biodegradable polymers. In one embodiment, the polymer is a biodegradable polymer. In some embodiments, the polymer is a hydrophobic polymer. In one embodiment, the aromatic compound is a catechol or a derivative thereof and the polymer contains reactive functional groups. In a preferred embodiment, the polymer is a polyanhydride that includes moieties of DOPA, a catechol derivative. These materials display bioadhesive properties superior to conventional bioadhesives used in therapeutic and diagnostic applications. In certain embodiments, the bioadhesive coating swells slightly and adheres to the mucosa in the aqueous environment of the gastrointestinal tract. As a result, the bioavailability of therapeutic agents is enhanced through increased residence time at the target absorption site. In certain embodiments, the bioadhesive dosage formulations described herein maintain a constant surface area for release of therapeutic agents at the target site.

### BRIEF DESCRIPTION OF THE DRAWINGS

FIG. 1 is a bar graph showing the fracture strength of bonds (mN/cm²) formed with the bioadhesive materials, Spheromer™ II and Spheromer™ III, as compared to Carbopol 934P and Gantrez AN polymers and control (uncoated substrate).

FIG. 2 is a bar graph of the tensile work (nJ) required to rupture the bonds formed with the bioadhesive materials, Spheromer™ II and Spheromer™ III, as compared to Carbopol 934P and Gantrez AN polymers and control (uncoated substrate).

FIG. 3 is a longitudinal section of a longitudinally compressed tablet containing a drug and excipients, and optionally permeation and/or dissolution enhancers, combined in a single monolithic layer. The tablet is coated peripherally with a bioadhesive, such as Spheromer™ II (anhydride oligomers blended with pharmaceutical polymers).

FIG. 4 is a longitudinal section of a longitudinally compressed tablet containing two different drugs (or same drug in different amounts) and excipients, and optionally permeation and/or dissolution enhancers, composed in two monolithic layers. Each layer contains one drug. The tablet is coated peripherally with a bioadhesive polymer, such as Spheromer™ II (anhydride oligomers blended with pharmaceutical polymers).

FIG. 5 is a longitudinal section of a longitudinally compressed tablet containing drugs and excipients, and optionally permeation and/or dissolution enhancers, disposed in four monolithic layers. The tablet is coated peripherally with a bioadhesive polymer, such as Spheromer™ II (anhydride oligomers blended with pharmaceutical polymers).

FIG. 6 is a longitudinal section of a longitudinally compressed tablet containing drugs and excipients, and optionally permeation and/or dissolution enhancers, disposed in two monolithic layers with a slow dissolving or insoluble plug at one end. The tablet is coated peripherally with a bioadhesive polymer, such as Spheromer™ II (anhydride oligomers blended with pharmaceutical polymers).

FIG. 7 is a longitudinal section of a longitudinally compressed tablet containing drugs and excipients, and optionally permeation and dissolution enhancers, disposed in two monolithic layers, separated by one or more slow-dissolving passive matrices, with an insoluble plug at one end. The tablet is coated peripherally with a bioadhesive polymer, such as Spheromer™ II (anhydride oligomers blended with pharmaceutical polymers).

FIG. 8 is a longitudinal section of a longitudinally compressed tablet containing drugs and excipients, and optionally permeation and dissolution enhancers, disposed in two monolithic layers, which are separated by a fast-dissolving passive matrix. The tablet is coated peripherally with a bioadhesive polymer, such as Spheromer™ II (anhydride oligomers blended with pharmaceutical polymers).

FIG. 9 is a longitudinal section of a longitudinally compressed tablet containing drugs and excipients, and optionally permeation and/or dissolution enhancers, disposed in two monolithic layers, which are separated by a fast-dissolving active matrix containing one or more active drugs. The tablet is coated peripherally with a bioadhesive polymer, such as Spheromer™ II (anhydride oligomers blended with pharmaceutical polymers). The active matrix is left unsealed.

FIG. 10 is a longitudinal section of a longitudinally compressed tablet containing drugs and excipients, and optionally permeation and/or dissolution enhancers, combined in a single matrix in which three pre-compressed reservoirs of drugs are embedded. The tablet is coated peripherally with a bioadhesive polymer, such as Spheromer™ II (anhydride oligomers blended with pharmaceutical polymers).

FIG. 11 is a longitudinal section of a longitudinally compressed tablet that functions as an osmotic delivery system. Drugs and excipients, optionally including permeation and/or dissolution enhancers, are combined in a single core matrix. The periphery of the tablet is coated first entirely coated with a semi-permeable membrane, and then partially or completely coated with a bioadhesive polymer, such as Spheromer™ II (anhydride oligomers blended with pharmaceutical polymers). One side of the tablet has a micrometer-sized orifice or exit port for drug release.

FIG. 12 is a longitudinal section of a longitudinally compressed tablet that functions as a “push-pull” osmotic delivery system. The core contains one layer of drugs and another layer of swelling polymer to push the drug out of the tablet at controlled rates. Permeation and/or dissolution enhancers are optionally added to the core. The periphery of the tablet is first entirely coated with a semi-permeable membrane, and then partially or completely coated with a bioadhesive polymer, such as Spheromer™ II (anhydride oligomers blended with pharmaceutical polymers).

FIG. 13 is a longitudinal section of a longitudinally compressed tablet containing drugs and excipients, and optionally permeation and/or dissolution enhancers, combined in one monolithic layer, plugged from the two ends by slow dissolving passive matrices. The tablet is coated peripherally with a bioadhesive polymer, such as Spheromer™ II (anhydride oligomers blended with pharmaceutical polymers).

FIG. 14 is a longitudinal section of a longitudinally compressed tablet that functions as an osmotic delivery system. Drugs and excipients, optionally including permeation and/or dissolution enhancers, are combined in a single core matrix. The periphery of the tablet is first entirely coated with a semi-permeable membrane, and then partially or completely coated with a bioadhesive polymer, such as Spheromer™ II (anhydride oligomers blended with pharmaceutical polymers).

FIG. 15 is a longitudinal section of a longitudinally compressed tablet containing drugs and excipients, and optionally permeation and/or dissolution enhancers, disposed in multiparticulates embedded in a single monolithic layer. Particulates are optionally coated with release rate controlling polymer(s). The matrix may be optionally a passive matrix. The tablet is coated peripherally with a bioadhesive polymer, such as Spheromer™ II (anhydride oligomers blended with pharmaceutical polymers).

FIG. 16 is a graph showing the in vitro release profile of Valacyclovir Tablets, 250 mg (Lot #502-094).

FIG. 17 is a graph showing the in vitro release profile of Gabapentin Tablets, 400 mg in USP simulated gastric fluid conditions (Lots 411-104, 412-047, 412-006).

FIG. 18 is a graph showing the in vitro release profile of Gabapentin Tablets, 500 mg in USP simulated gastric fluid conditions (Lots 411-108, 411-106, 411-029).

FIG. 19 is a graph showing the in vitro release profile of Valacyclovir Tablets, 225 mg in USP simulated gastric fluid conditions (Lots 502-063, 502-065, 502-067).

FIG. 20 is a graph showing the in vitro release profile of Valacyclovir Tablets, 450 mg in USP simulated gastric fluid conditions (Lots 502-069, 502-071, 502-073).

FIG. 21 is a graph showing the in vitro dissolution of Metformin HCl tablets, 500 mg under USP simulated gastric fluid conditions (Lot #412-093).

FIG. 22 is a graph showing the in vitro dissolution of Pioglitazone HCl tablets, 30 mg under USP simulated gastric fluid conditions (Lot #503-036).

FIG. 23 is a graph showing the in vitro dissolution of Pioglitazone HCl tablets, 30 mg under USP simulated gastric fluid conditions (Lot #503-203).

FIG. 24 is a graph showing the in vitro release profile of Valacyclovir Tablets, 450 mg in USP simulated gastric fluid conditions (Lot #501-213).

FIG. 25 is a graph showing the in vitro dissolution of Glipizide-Metformin HCl tablets under USP simulated intestinal buffer (Lot #502-105).

FIG. 26 is a graph showing the in vitro dissolution of Gabapentin-Sodium Valproate tablets under USP simulated intestinal buffer pH 6.8 (Lot #412-010).

FIG. 27 is a graph showing the in vitro dissolution of Rosiglitazone-Metformin HCl tablets, 4 mg/500 mg under USP simulated gastric buffer containing additional KCL (Lot #503-167).

FIG. 28 is a graph showing the in vitro dissolution of Rosiglitazone HCl-Metformin HCL tablets, 4 mg/500 mg under USP simulated gastric fluid conditions (Lot #504-060).

FIG. 29 is a graph showing the in vitro dissolution of Pioglitazone HCl tablets, 30 mg under USP simulated gastric fluid conditions (Lot #503-038).

FIG. 30 is a graph showing the in vitro dissolution of three different lots of Pioglitazone HCl tablets, 45 mg under USP simulated gastric fluid conditions (Lot #502-045).

FIG. 31 is a graph showing the in vitro dissolution profile of Valacyclovir tablets under USP simulated gastric fluid conditions. (Lot #505-018).

FIG. 32 is a graph showing the in vitro dissolution profile of Valacyclovir tablets, 250 mg under USP simulated gastric fluid conditions (Lot #504-027).

FIG. 33 is a graph showing the in vitro dissolution profile of two different lots of Valacyclovir tablets, 250 mg, under USP simulated gastric fluid conditions (Lots #504-079, 504-081).

FIG. 34 is a longitudinal section of a longitudinally compressed tablet containing drugs and excipients, and optionally permeation and/or dissolution enhancers, disposed in three monolithic layers. The tablet is coated peripherally with a bioadhesive polymer, such as Spheromer™ II (anhydride oligomers blended with pharmaceutical polymers). Drug is released from one end of the tablet in this design.

FIG. 35 is a longitudinal section of a longitudinally compressed tablet that functions as a “push-rod” osmotic delivery system. The core contains one layer of drugs and another layer of swelling polymer to push the drug out of the tablet at controlled rates. Permeation and/or dissolution enhancers are optionally added to the core. The periphery of the tablet is first partially coated with a semi-permeable membrane, and then partially coated with a bioadhesive polymer, such as Spheromer™ II (anhydride oligomers blended with pharmaceutical polymers).

FIG. 36 is a longitudinal section of a longitudinally compressed tablet containing drugs, excipients, and bioadhesive polymer, and optionally permeation and/or dissolution enhancers, disposed in multiparticulates embedded in a single monolithic layer. The matrix may be optionally a passive matrix. The tablet is coated peripherally with a bioadhesive polymer, such as Spheromer™ II (anhydride oligomers blended with pharmaceutical polymers).

FIG. 37 is a longitudinal section of a longitudinally compressed tablet containing drugs and excipients, and optionally permeation and/or dissolution enhancers, disposed in multiparticulates embedded in a single monolithic layer. Particulates are coated with an enteric coating or other rate-controlling polymer. The matrix may be optionally a passive matrix. The tablet is coated peripherally with a bioadhesive polymer, such as Spheromer™ II (anhydride oligomers blended with pharmaceutical polymers).

FIG. 38 is a longitudinal section of a longitudinally compressed tablet containing drugs and excipients, and optionally permeation and/or dissolution enhancers, disposed in multiparticulates embedded in a single monolithic layer. Particulates are coated with bioadhesive polymer, which is further coated with an enteric coating or other rate-controlling polymer. The matrix may be optionally a passive matrix. The tablet is coated peripherally with a bioadhesive polymer, such as Spheromer™ II (anhydride oligomers blended with pharmaceutical polymers).

FIG. 39 is a longitudinal section of a bullet-shaped inner core containing drugs and excipients, and optionally permeation and/or dissolution enhancers. The inner core is coated peripherally with a bioadhesive polymer, such as Spheromer™ II (anhydride oligomers blended with pharmaceutical polymers).

FIG. 40 is a graph showing the in vitro dissolution of Levodopa—Carbidopa tablets, 200 mg/50 mg under USP simulated gastric fluid conditions (Lot #506-047).

FIG. 41 is a graph showing the in vitro dissolution of Levodopa—Carbidopa tablets, 200 mg/50 mg under USP simulated gastric fluid conditions (Lot #507-041).

### DETAILED DESCRIPTION OF THE INVENTION

Described herein are bioadhesive materials that may be used in a wide variety of drug delivery and diagnostic applications. In preferred embodiments, the bioadhesive material is applied as a coating to any longitudinally compressed core, which forms the inner core of a bioaadhesive, rate-controlled oral dosage formulation. The term “rate-controlled” refers to controlling the release of an agent to provide a pre-determined release profile of each agent. For example, a drug can be released over a period of 1 hour, 2 hours, or more than 3 hours and/or can be released after a period of 1 hour, 2 hours, or more than 3 hours. The inner core may comprise a therapeutic, prophylactic, or diagnostic agent and may further comprise excipients, permeation enhancers, and/or dissolution enhancers.

In one embodiment, the present invention is an oral dosage form, such as a tablet, for oral delivery of a drug, comprising a compressed core including a drug to be delivered gastrointestinally, and a bioadhesive polymeric coating applied to at least one surface of the dosage form. The coating preferably provides the dosage form with a fracture strength of at least 100 N/m² as measured on rat intestine, and the dosage form optimally has a gastrointestinal retention time of at least 4 hours in a fed beagle dog model during which the drug is released from the dosage form. In another embodiment, the dosage form optimally has a gastrointestinal retention time of at least 3 hours in a fasted beagle dog model during which the drug is released from the dosage form. In certain embodiments, the bioadhesive polymer coating further includes metal compounds, low molecular weight oligomers or a combination thereof that enhance the mucosal adhesion of the synthetic polymer coating. In preferred embodiments, a dosage form is prepared by extrusion of a bioadhesive cylinder followed by insertion of an inner core composition and fusing the two units together.

In another embodiment, the invention is a drug eluting device for oral delivery of a drug, which includes a reservoir having a drug-containing compressed core contained therein, one or more orifices or exit ports through which drug from the core can elute from the device, and a bioadhesive polymeric coating, applied to at least one surface of the device. The coating preferably provides the device with a fracture strength of at least 100 N/m² as measured on rat intestine, and the device optimally has a gastrointestinal retention time of at least 4 hours in a fed beagle dog model during which the drug is released from the device. In another embodiment, the tablet optimally has a gastrointestinal retention time of at least 3 hours in a fasted beagle dog model during which the drug is released from the device. In certain embodiments, the bioadhesive polymer coating further includes metal compounds, low molecular weight oligomers or a combination thereof that enhance the mucosal adhesion of the synthetic polymer coating. In a preferred embodiment, the bioadhesive polymeric coating does not substantially swell upon hydration.

In another embodiment, the present invention is an orally administrable, multi-layer, pharmaceutical tablet having an inner and one or more outer layers, each comprising a drug (e.g., a drug including a valproic moiety such as sodium valproate, divalproex sodium, valproic acid, etc.) admixed with one or more excipients, and a bioadhesive polymeric coating applied to at least one surface of the tablet. At least one of the excipients is hydrophobic, although such excipient is not required in each layer. Additional outer layers (i.e., layers other than the inner and outer layers specified above) are optionally free of the drug.

The present invention provides methods for improving the bioadhesive properties of drug delivery systems such as tablets and drug eluting devices. The invention also provides methods for improving the adhesion of drug delivery systems to mucosal membranes including membranes of the gastrointestinal tract. The polymeric drug delivery systems of the invention have an improved ability to bind to mucosal membranes, which can be used to deliver a wide range of drugs or diagnostic agents in a wide variety of therapeutic applications, and have an improved ability for uptake across the intestinal mucosa.

### Bioadhesive Oral Drug Delivery Formulations

BIOROD

Bioadhesive materials described herein may be used in a wide variety of drug delivery and diagnostic applications. Bioadhesive materials may be formed into microparticles, such as microspheres or microcapsules, or may be a coating on such microparticles. In preferred embodiments, the material is applied as a coating to any longitudinally compressed tablet.

The overall shape of the device is typically designed to be compatible with swallowing. For example, in one embodiment, the active agent core is longitudinally compressed to form a capsule-shaped tablet, which is encapsulated and sealed in a bioadhesive polymeric cylinder. The compressed tablet may comprise one or more than one active agent and may further comprise excipients and absorption enhancers (e.g., permeation and/or dissolution enhancers).

The subject formulas are suitable for the delivery of any kind of therapeutic, prophylactic or diagnostic agent in a wide variety of immediate and controlled-release formulations. Also, the subject invention provides for the delivery of more than one drug in a single dosage formulation. For example, different drugs may be included in one heterogenous layer of a solid dosage formulation. Alternatively, different drugs, for example, a highly aqueous-soluble drug and a poorly aqueous-soluble drug, may be included in discrete layers of a compressed, multilayer solid dosage formulation, such as a compressed, multilayer tablet. In some instances, a compressed, multilayer tablet, for example, may comprise a combination of homogeneous and heterogenous drug layers, such as a bilayer tablet that comprises one drug in one layer and a blend of two different drugs in the second layer.

The different drug formulations in each layer may be designed such that the drug release profiles are the same or differ to one another. For example, single dosage forms of the invention can comprise more than one drug (e.g., two), wherein each drug is formulated for release in a manner that differs to the other drug, such as dosage formulations that comprise a combination of immediate release, controlled release, and/or ascending or descending drug release formulations. In such embodiments, each individual drug may be segregated in its own layer, or two or more drugs may be present in the same layer. Even in the latter arrangement, different release rates may be possible, for example, if one drug is present as a soluble powder and the other is present in particles, which may be formulated to be slow releasing or coated with a rate-controlling membrane. Each layer may further be coated with one or more coatings or layers (e.g., polymers or other suitable materials), such as an impermeable layer that restricts release to certain defined ports, a permeable layer that controls the rate of release through the layer, a bioadhesive coating that may slow passage of that layer independent of other layers in the dosage form, or any combination thereof.

In some embodiments, a drug or drugs may be formulated for release in a pulsatile fashion. For example, two or more immediate-release formulations of a drug may be embedded within a slow-dissolving matrix, such that drug is released (e.g., in bursts) at discrete intervals as the surrounding matrix dissolves over time.

In any layer, the active agent may be dispersed evenly throughout, e.g., mixed as a powder with any excipients and additives to form a uniform, monolithic layer, or may be present as particles or granules dispersed in an excipient or other matrix. In addition to the active agent, layers may contain standard pharmaceutically acceptable excipients, biocompatible polymers (e.g., for controlling the rate of release), dispersants, disintegrants, superdisintegrants, or other additives that modify the physical, chemical, or biological properties of the layer. A layer may further contain additives that enhance the effect of the agents in that layer, such as permeability enhancers, efflux pump inhibitors, solubilizing agents, and other such adjuvants.

The solid dosage formulations of the invention preferably comprise a compressed inner core, on which bioadhesive polymer is disposed on at least one surface. It is expected that the bioadhesive polymer will promote retention of the dosage formulation at a target site of absorption in the gastrointestinal tract. The bioadhesive polymer can be applied in any suitable way. Preferably, a pre-compressed core of the invention is inserted into a bioadhesive polymer tube. Various embodiments of the instant invention are described in greater detail below.

One of skill in the art will understand that, as the term is used in this application, that when a surface is “coated” with a layer, it is not necessary that the entire surface be covered, only that at least a portion of that surface bears the specified layer, unless the context indicates that entire surface must be covered to achieve the desired effect.

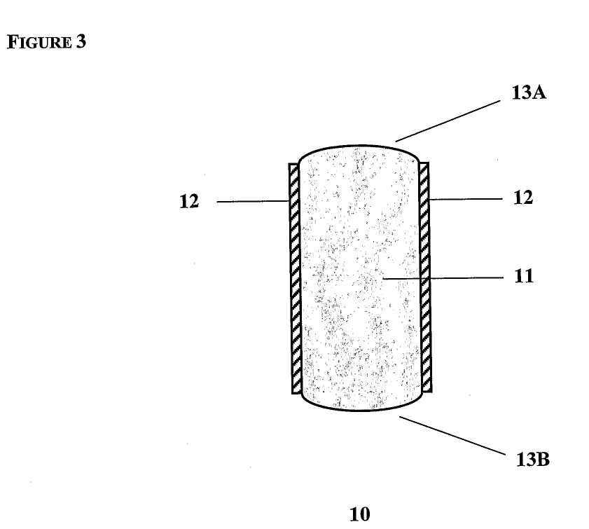

In a preferred embodiment, illustrated in FIG. 3, the solid oral dosage form is a longitudinally compressed tablet 10 containing a single drug or more than one drug, excipients, and optionally permeation and/or dissolution enhancers, combined in a single monolithic layer 11. The tablet is sealed peripherally with a layer of bioadhesive composition 12 leaving the upper and lower sides 13A and 13B of the tablet available for drug release. First-order and, more advantageously, zero-order release profiles are achievable with this tablet design. It is feasible to create different drug release rates by changing the composition of the core matrix.

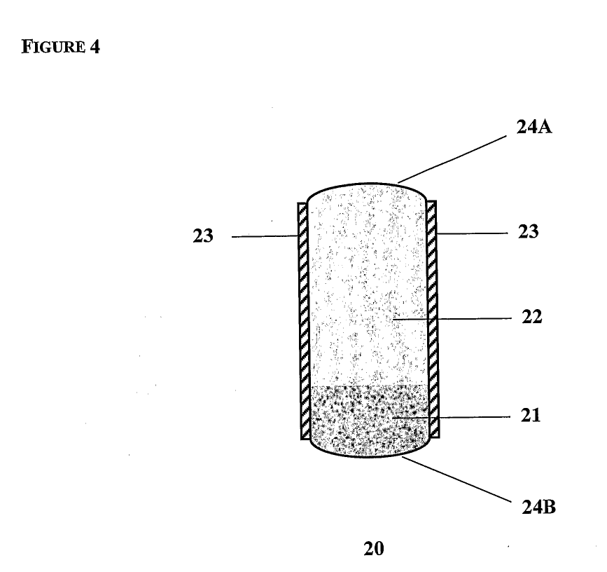

In another embodiment, illustrated in FIG. 4, the solid oral dosage form is a longitudinally compressed tablet 20 containing a drug or two different drugs or two different compositions of the same drug (e.g., concentration gradient), excipients, and optionally permeation and/or dissolution enhancers, disposed in two monolithic layers 21 and 22. The tablet is sealed peripherally with a layer of bioadhesive composition 23 leaving the upper and lower sides 24A and 24B of the tablet available for drug release. First-order and, more advantageously, zero-order release profiles are achievable with this tablet design. In this embodiment, the tablet can be designed to provide immediate release (IR) of one or more soluble, poorly soluble or insoluble drugs from one layer and extended release (ER) of one or more soluble drugs from the other layer. Various drug release rates can be achieved by changing the composition and/or configuration of IR and ER matrices.

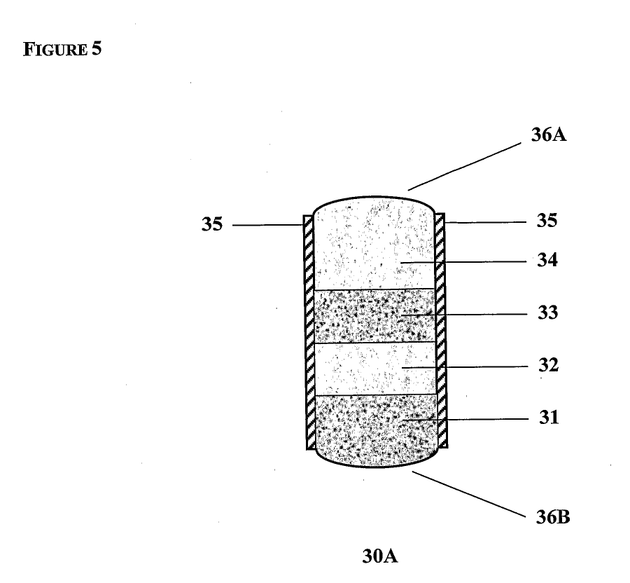

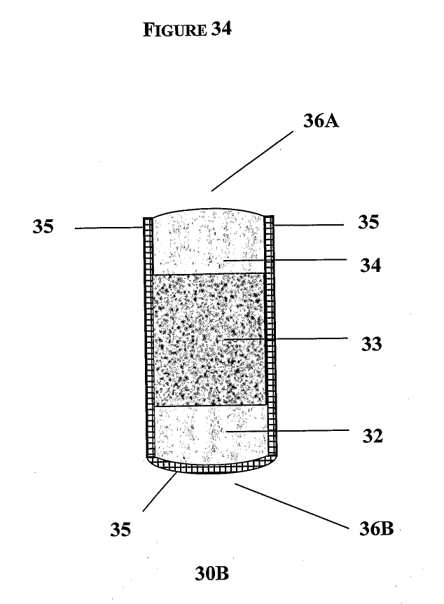

In another embodiment, illustrated in FIG. 5, the solid oral dosage form is a longitudinally compressed tablet 30A containing a single drug, two or more different drugs, or two different concentrations of a single drug, in combination with excipients, and optionally permeation and/or dissolution enhancers, in a single monolithic layer (not shown) or multiple monolithic layers 31-34. The tablet is sealed peripherally with a layer of bioadhesive composition 35 leaving the upper and lower sides 36A and 36B of the tablet available for drug release. First-order and, more advantageously, zero-order release profiles are achievable with this tablet design. The tablet can be designed to provide various extended release (ER) rates for the drugs by changing the composition of the core matrix or by changing the configuration of their respective layers. In a related embodiment, illustrated in FIG. 34, the tablet 30B is sealed peripherally with a layer of bioadhesive composition 35 leaving only the upper side 36A of the tablet available for drug release.

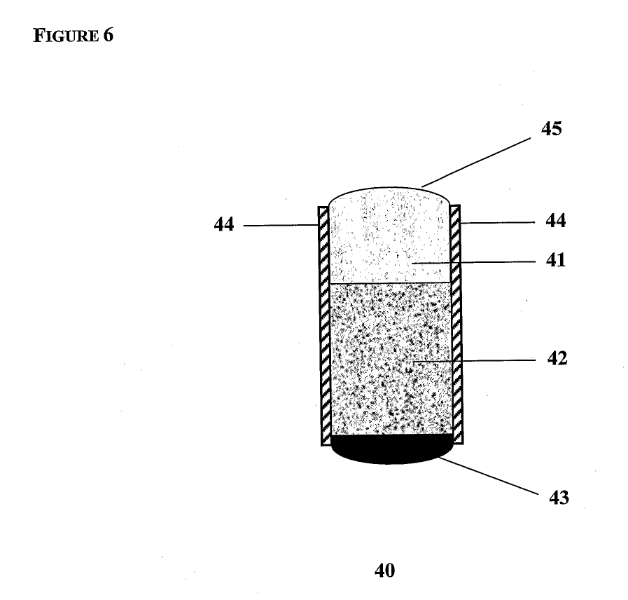

In another embodiment, illustrated in FIG. 6, the solid oral dosage form is a longitudinally compressed tablet 40 containing one or more drugs, excipients, and optionally permeation and/or dissolution enhancers, disposed in two or more monolithic layers 41 and 42 blocked at one end by a slow-dissolving or non-dissolving passive matrix (also referred to herein as “plug”) 43. The tablet is coated peripherally with a layer of bioadhesive composition 44 leaving the upper side 45 of the tablet available for drug release. First-order and, more advantageously, zero-order release profiles are achievable with this tablet design. The tablet can be designed to provide different immediate release or extended release rates for drugs by changing the composition of the drug layers, or by changing the formulation of the plug.

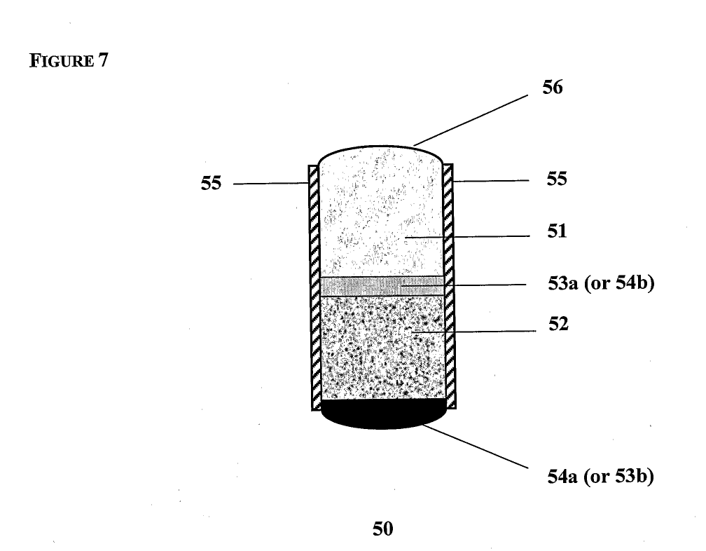

In another embodiment, illustrated in FIG. 7, the solid oral dosage form is a longitudinally compressed tablet 50 containing two drugs, excipients, and optionally permeation and/or dissolution enhancers, disposed in two or three (not shown) 51 and 52 monolithic layers which are separated by one or more slow dissolving passive matrices 53a and blocked from one end by one insoluble plug 54a. Alternatively, two monolithic layers 51 and 52 containing different drugs can be separated by one insoluble plug 54b and blocked from one end by one slow dissolving passive matrix 53b. The tablet is coated peripherally with a layer of bioadhesive composition 55 leaving the upper side 56 of the tablet available for drug release. First-order and, more advantageously, zero-order release profiles are achievable with this tablet design. The tablet can be configured to provide different immediate release or extended release rates for drugs in a two-pulse or three-pulse fashion by changing the composition or configuration of the drug layers, or by changing the formulation or configuration of the slow dissolving matrices and the insoluble plug.

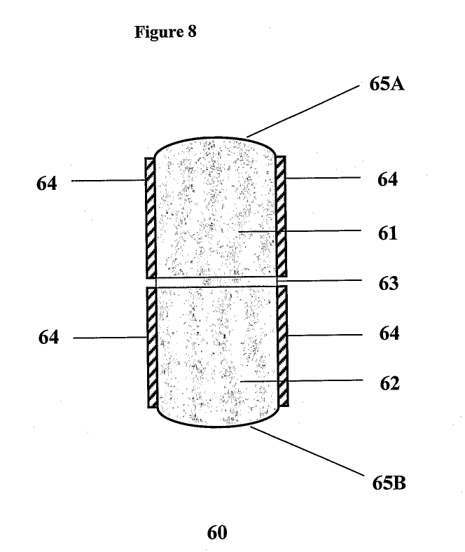

In the embodiment illustrated in FIG. 8, the solid oral dosage form is a longitudinally compressed tablet 60 containing one or more drugs, excipients, and optionally permeation and/or dissolution enhancers, disposed in two or three (not shown) monolithic layers 61 and 62 which are separated by one or more fast dissolving passive matrices 63. The tablet is coated peripherally with a bioadhesive composition 64 sealing the drug layers while leaving the passive matrice(s) unsealed. The upper and lower sides of the tablet 65A and 65B are available for drug release. The tablet splits into two or more segments upon the complete dissolution of the fast dissolving passive matrix 63, thereby creating new surfaces for dissolution, resulting in an increased rate of drug release from layers 61 and 62.

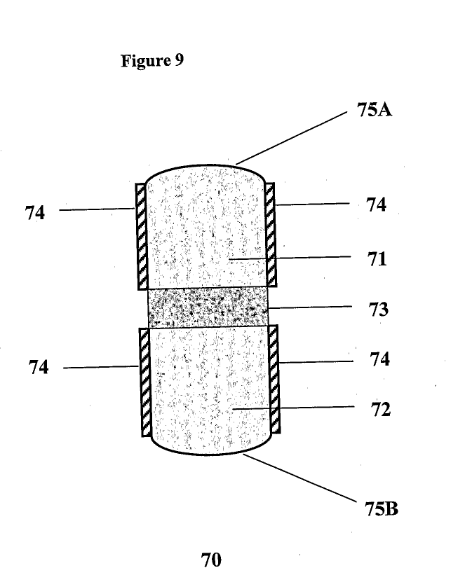

In another embodiment, illustrated in FIG. 9, the solid oral dosage form is a longitudinally compressed tablet 70 containing two or more different drugs, excipients, and optionally permeation and/or dissolution enhancers, composed in two or three (not shown) monolithic layers 71 and 72 which are separated by one or more drug-containing layers 73 the latter being capable of providing an immediate release (IR) of one or more soluble, poorly soluble or insoluble drugs. The tablet is coated peripherally with a bioadhesive composition 74 sealing the drug layers 71 and 72, while leaving the IR layer 73 unsealed. The upper and lower sides of the tablet 75A and 75B are available for drug release. The tablet splits into two or more segments upon the complete dissolution of the middle IR layer 73, thereby creating new surfaces for dissolution, resulting in an increased rate of drug release from layers 71 and 72.

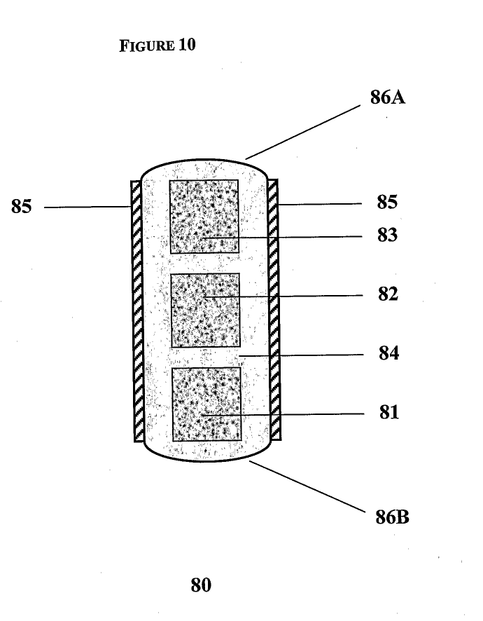

In yet another embodiment, illustrated in FIG. 10, the delivery system is a longitudinally compressed tablet 80 containing pre-compressed inserts 81-83 of a drug or two drugs, excipients, and optionally permeation and/or dissolution enhancers embedded in a matrix 84 of drugs, excipients, and optionally permeation and/or dissolution enhancers. The matrix may optionally be a passive matrix. The tablet is coated peripherally with a layer of bioadhesive composition 85 leaving the upper and lower edges 86A and 86B of the tablet available for drug release. The kinetics of drug release are controlled by the geometry of the inserts. Zero- and first-order release profiles are achievable with this tablet design, and it is possible to have different release rates for permeation enhancer and drug by changing the configuration of their respective inserts.

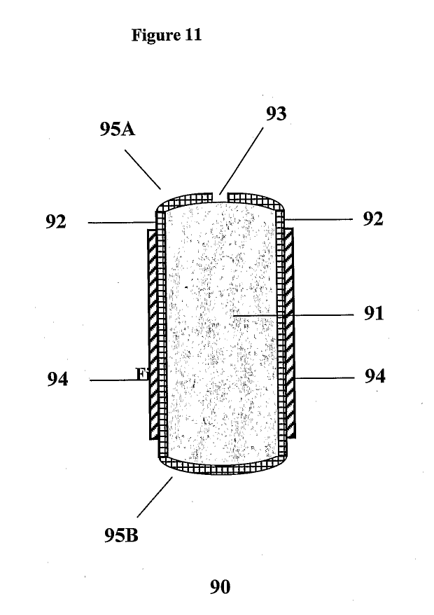

In still yet another embodiment, the drugs are delivered from an elementary osmotic delivery system. FIG. 11 illustrates the cross section of a longitudinally compressed tablet 90 based on osmotic controlled delivery containing drugs, excipients, and optionally permeation and/or dissolution enhancers, composed in a single core matrix 91. The tablet is coated with a semipermeable membrane 92. One or both sides of the tablet may be perforated, such as by using a micro-drill or a laser beam to make a micrometer-sized orifice 93. The tablet is sealed peripherally with a matrix of bioadhesive composition 94 leaving the orifice 93 and upper and/or lower sides 95A and 95B of the tablet available for water uptake. The semi-permeable membrane allows permeation of water into the matrix, leading to the dissolution of drug and creation of osmotic pressure. An increase of osmotic pressure will push the drug slurry out of the device through the one or more orifice(s) and membrane at controlled rates. Zero-order release profiles are achievable with this tablet design. The osmotic system may also be designed in such a manner that the drug release is from the entire surface 95A, and this system is referred to herein as a “Push Rod” system.

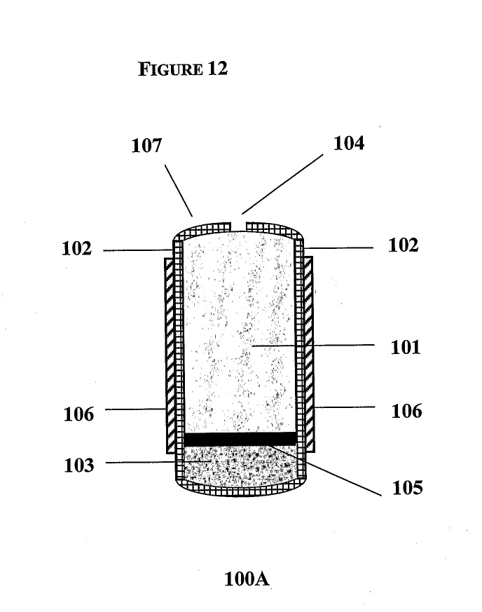

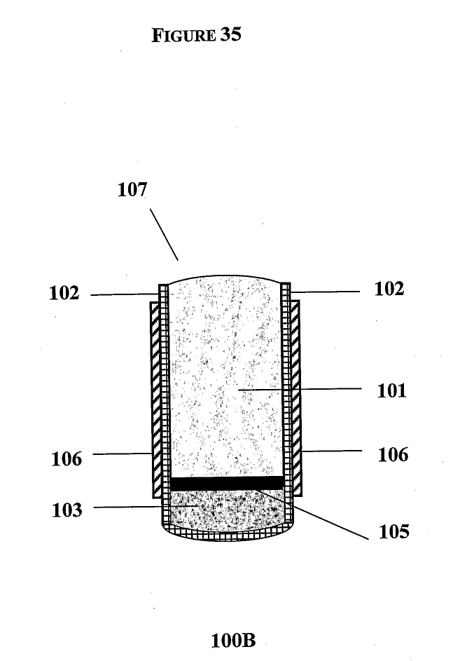

A cross section of an osmotic delivery system is illustrated in FIG. 12. The osmotic delivery system is of the “push-pull” design 100A and contains drugs and osmotic agents 101 to draw water across a semi-permeable membrane 102 and a swelling polymer 103 to push the drug out of the device at controlled rates. Drug sides of the tablet may be perforated, such as by using a micro-drill or a laser beam to make a micrometer-sized orifice 104. Optionally, the drug layer 101 is separated from the push layer 103 by an “insoluble plug” 105. The purpose of this insoluble plug is to ensure that there is complete drug release from the system. In a traditional “push-pull system”, the drug may get trapped within the swollen push layer resulting in high residual drug in the system. The device is peripherally coated with a bioadhesive composition 106, leaving the orifice 104, the upper edge 107, and lower edge of the tablet available for drug release. In a related embodiment, illustrated in FIG. 35, the osmotic delivery system is of a “push-rod” design 100B. In this embodiment, the upper edge 107 is not covered by the semi-permeable membrane 102.

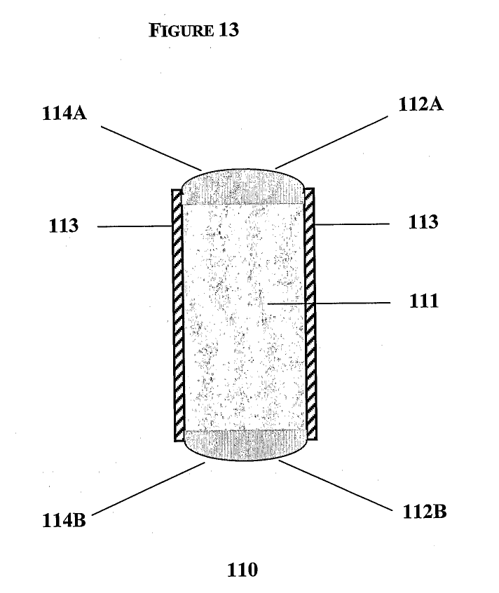

In another embodiment, illustrated in FIG. 13, the solid oral dosage form is a longitudinally compressed tablet 110 containing one or more drugs, excipients, and optionally permeation and/or dissolution enhancers, combined in one monolithic layer 111 which is plugged from the two ends by slow eroding passive matrices 112A and 112B. The tablet is coated peripherally with a layer of bioadhesive composition 113 leaving the upper and lower sides 114A and 114B of the tablet available for dissolution. Upon dissolution of the slow eroding passive layers 112A and 112B the active layer 111 is exposed to the surrounding environment, releasing the drug in an immediate release or extended release mode. The tablet can be designed to release its contents in a time-controlled manner with different delayed periods by changing the composition of the passive layers.

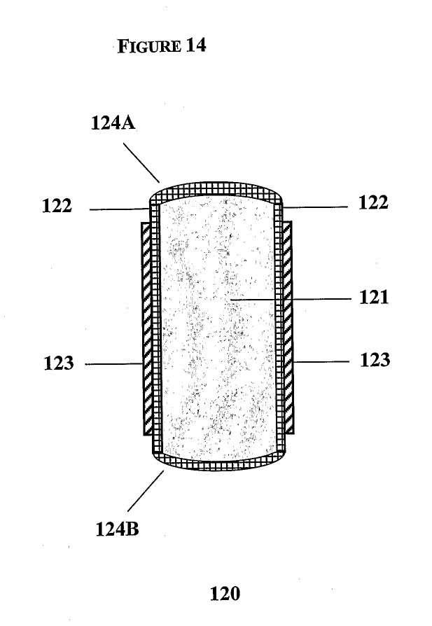

In another embodiment, the one or more drugs are delivered from a microporous/macroporous based osmotic delivery system. FIG. 14 illustrates the cross-section of a longitudinally compressed tablet 120 containing one or more drugs, excipients, and optionally permeation and/or dissolution enhancers, composed in a single core matrix 121. The tablet is coated with a semi-permeable membrane 122 comprising a hydrophobic polymer and one or more hydrophilic pore formers or wicking agents. The tablet is sealed peripherally with a matrix of bioadhesive composition 123 leaving the upper and lower sides 124A and 124B of the tablet available for water uptake and drug release. The hydrophilic pore formers or wicking agents in the semi-permeable membrane 122 dissolve in an aqueous environment, creating micro- or macro-pores or channels on the semi-permeable membrane. The micro- or macro-porous membrane allows permeation of water into the matrix, leading to the dissolution of drug and creation of osmotic pressure. The increase of osmotic pressure will push the drug out of the device through the micro- or macrochannels at controlled rates. Zero-order release profiles are achievable with this tablet design.

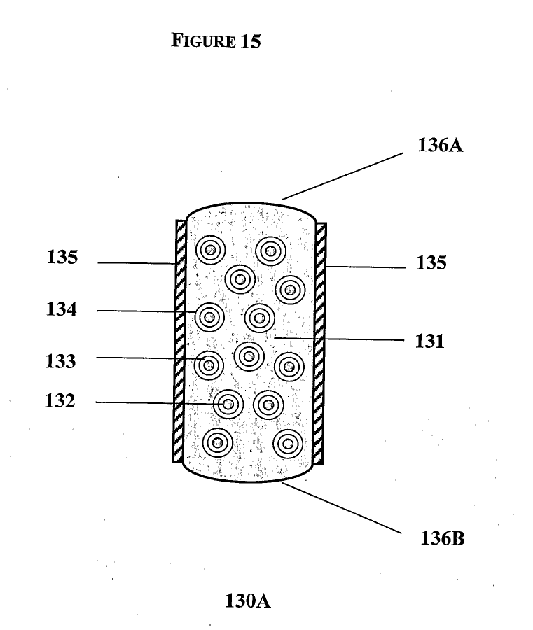

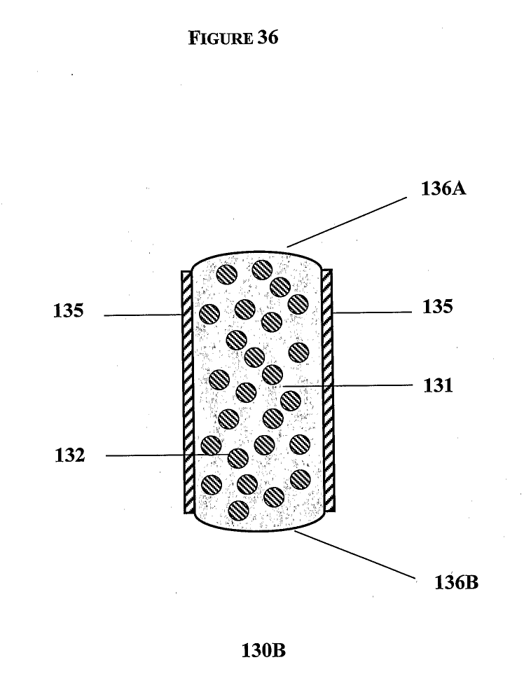

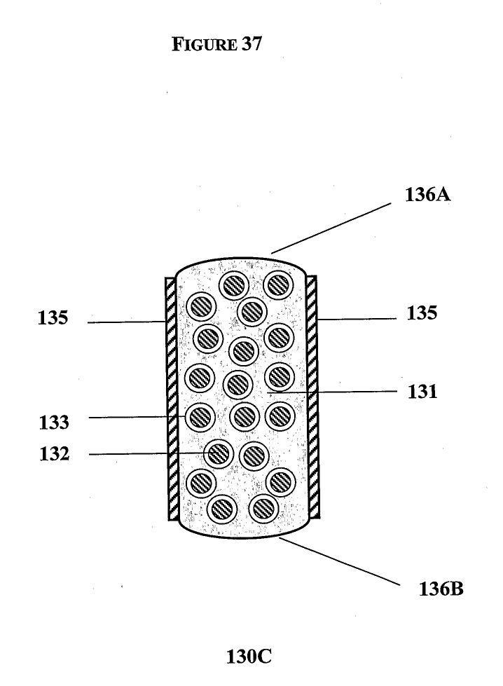

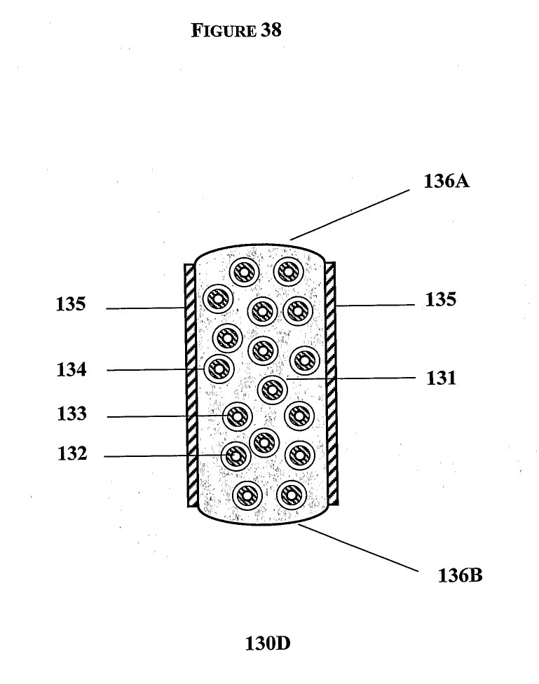

In yet another embodiment, illustrated in FIG. 15, the solid oral dosage form is a longitudinally compressed tablet 130A comprising one or more drugs, excipients, and optionally permeation and/or dissolution enhancers, in a single monolithic matrix 131. The matrix contains a plurality of particles 132 of a drug in combination with excipients, and optionally permeation and/or dissolution enhancers. The said particles are optionally coated with one or more layers of release rate-controlling polymers 133 and 134. The matrix may be optionally a passive matrix containing a filler and “cushioning component”. A cushioning material would preferentially absorb the forces exerted during the compaction of the inner core, thereby providing protection to the coated particles. The tablet is sealed peripherally with a layer of bioadhesive composition 135 leaving the upper and lower sides 136A and 136B of the tablet available for drug release. First-order and, more advantageously, zero-order release profiles are achievable with this tablet design. It is feasible to create different release rates for drugs by changing the composition of the core matrix, embedded core particles, or rate-controlling polymer-coated multiparticles. In a related embodiment, illustrated in FIG. 36, the particles 132 are formulated with bioadhesive polymers. In some embodiments, illustrated in FIG. 37, the particles further comprise an enteric coating or other rate-controlling polymer 133. In another embodiment, illustrated in FIG. 38, the particles comprise one or more drugs and excipients 132 and are coated with a bioadhesive polymer 133, which is coated with an enteric or other rate-controlling polymer 134.

The particles can be micro- or nanoparticles. The term “microparticles” is art-recognized, and includes microspheres and microcapsules, as well as structures that may not be readily placed into either of the above two categories, all with dimensions on average of less than about 1000 microns. The term “microspheres” is art-recognized, and includes substantially spherical colloidal structures having a size ranging from about one or greater up to about 1000 microns. In general, “microcapsules”, also an art-recognized term, may be distinguished from microspheres, because microcapsules are generally covered by a substance of some type, such as a polymeric formulation, e.g., a bioadhesive coating or rate-controlling polymer layer. If the structures are less than about one micron in diameter, then the corresponding art-recognized terms “nanoparticle,” “nanosphere,” and “nanocapsule” may be utilized. In certain embodiments, the nanospheres, nancapsules and nanoparticles have an average diameter of about 500, 200, 100, 50 or 10 nm.

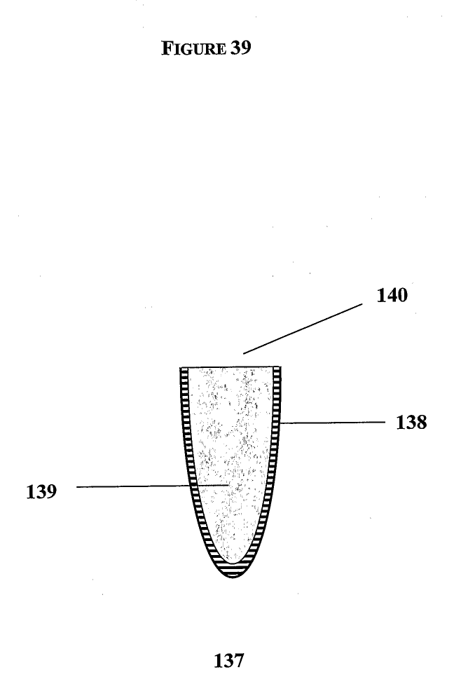

Other release rates that can be achieved with the dosage formulations of the present invention include ascending as well as descending drug release profiles. For example, in some embodiments, the inner core comprises two or more layers (e.g., a layer comprising the drug to be released and a “push” layer), from which the therapeutic agent is released at an increasing rate through an orifice or exit pore in the bioadhesive layer. In some embodiments, the bioadhesive layer is further coated with a drug formulation that dissolves quickly for immediate release of the drug while the drug disposed in the inner core is released at an escalating rate. In some embodiments, the geometry of the dosage formulation contributes to the ascending or descending drug release profile, for example, dosage formulations in a semi-ellipsoid shape (e.g., bullet-shaped). Such a dosage formulation is illustrated in FIG. 39.

In the embodiment illustrated in FIG. 39, the dosage form 137 is suitable as a suppository and comprises a bullet-shaped inner core 139 composed of one or more drugs, excipients, and optionally permeation and/or dissolution enhancers, combined in a single monolithic layer. The dosage formulation is sealed peripherally with a layer of bioadhesive composition 138, leaving the wide, upper side 140 of the dosage form available for drug release. As drug is released, the surface area of the inner core decreases, thereby releasing less drug over time. Thus, descending drug release profiles are achievable with this solid dosage form design. Alternatively, in some embodiments, the dosage formulation is sealed peripherally with a layer of bioadhesive composition, leaving the narrow, lower end available for drug release. In this embodiment, the surface area of the inner core increases as drug is released, thereby releasing increasing amounts of drug over time. Accordingly, ascending drug release profiles are achievable in this embodiment. In some embodiments, the inner core comprises more than one layer of drug, excipients and permeation and/or dissolution enhancers, e.g., a trilayer dosage form design.

### Drugs and Active Agents

A wide variety of drugs can be included in tablets and drug eluting devices of the invention. Such tablets and drug eluting devices typically contain at least 1 mg of a drug. These tablets and drug eluting devices can also contain at least 2 mg, at least 5 mg, at least 10 mg, at least 25 mg, at least 50 mg, at least 100 mg, at least 500 mg or at least 1000 mg of a drug (e.g., 2 mg to 1000 mg).

Drugs suitable for use herein can be any kind of therapeutic, prophylactic or diagnostic agent, including inorganic compounds, small organic molecules (e.g., non-polymeric molecules having a molecular weight of 2000 Da or less, such as 1000 Da or less), peptides or polypeptides, polysaccharides, and nucleic acids. Active agents suitable for use herein also include flavorants, nutraceuticals, and dietary supplements.

Drugs may be classified using the Biopharmaceutical Classification System (BCS), which separates pharmaceuticals for oral administration into four classes depending on their solubility and their absorbability through the intestinal cell layer. According to the BCS, drug substances are classified as follows:

Class I—High Permeability, High Solubility

Class II—High Permeability, Low Solubility

Class III—Low Permeability, High Solubility

Class IV—Low Permeability, Low Solubility

The interest in this classification system stems largely from its application in early drug development and then in the management of product change through its life-cycle. In the early stages of drug development, knowledge of the class of a particular drug is an important factor influencing the decision to continue or stop its development.

Class I drugs of the BCS system are highly soluble and highly permeable in the gastrointestinal (GI) tract.

Class II drugs are drugs that are particularly insoluble, or slow to dissolve, but that readily are absorbed from solution by the lining of the stomach and/or the intestine. Therefore, prolonged exposure to the lining of the GI tract is required to achieve absorption. Sometimes BCS Class II drugs may be micronized to sizes less than 2 microns to increase the rate of dissolution.

Many of the known Class II drugs are hydrophobic, and have historically been difficult to administer. Moreover, because of their hydrophobicity, there tends to be a significant variation in absorption depending on whether the patient is fed or fasted at the time of taking the drug. This in turn can affect the peak level of serum concentration, making calculation of dosage and dosing regimens more complex.

Class III drugs include biologic agents that have good water solubility and poor GI permeability, such as proteins, peptides, polysaccharides, nucleic acids, nucleic acid oligomers and viruses.

Class IV drugs are lipophilic drugs with poor GI permeability. Both Class III and IV drugs are often problematic or unsuitable for sustained release or controlled release. Class III and Class IV drugs are characterized by poor biomembrane permeability and are commonly delivered parenterally. Traditional approaches to parenteral delivery of poorly soluble drugs include using large volumes of aqueous diluents, solubilizing agents, detergents, non-aqueous solvents, or non-physiological pH solutions. These formulations, however, can increase the systemic toxicity of the drug composition or damage body tissues at the site of administration.

In one embodiment, one or more Class I, II, III, or IV drugs are included in a compressed core of a solid oral dosage formulation, and the core is surrounded on all or part of its exterior by one or more bioadhesive polymers.

In one example, the drug is selected from hormones, enzymes, antigens, digestive aids, ulcer treatments (e.g., bismuth subsalicylate optionally in combination with antibiotics effective against H. pylori), antihypertensives, enzyme inhibitors, antiparasitics (e.g., antimalarials such as atovaquone), spermicides, anti-hemorrhoidal treatments, and radiopaque compounds. In another example, the drug is an antifungal agent (e.g., itraconazole, fluoconazole, terconazole, ketoconazole, saperconazole, griseofulvin, griseoverdin). In a further example, the drug is an antineoplastic agent. In yet another example, the drug is an antiviral agent (e.g., acyclovir). Other classes of drugs suitable for inclusion in tablets and drug eluting devices of the invention include steroids (e.g., danazol), immunosuppressants (e.g., cyclosporine), CNS active agents, cardiovascular agents, anti-depressant agents, anti-psychotic agents, anti-epileptic agents (e.g., carbamazepine), agents for treating a movement disorder (e.g., valproic acid and salts thereof) and anti-migraine agents (e.g., triptans such as sumatriptan).

Preferred materials to be incorporated into the compressed tablets or drug eluting devices are drugs and imaging agents. Suitable drugs include antibiotics, antivirals (especially protease inhibitors alone or in combination with nucleosides for treatment of HIV or Hepatitis B or C), anti-parasitics (helminths, protozoans), anti-cancer agents (referred to herein as “chemotherapeutics”, including cytotoxic drugs such as cisplatin and carboplatin, BCNU, 5FU, methotrexate, adriamycin, camptothecin, and taxol), anti-TNF (tumor necrosis factor) agents, antibodies and bioactive fragments thereof (including humanized, single chain, and chimeric antibodies), antigen and vaccine formulations, peptide drugs, anti-inflammatories, and oligonucleotide drugs (including antisense, aptamers, ribozymes, external guide sequences for ribonuclease P, and triplex forming agents).

Other suitable drugs include valacyclovir, gabapentin, metformin, pioglitazone, glipizide, rosiglitazone, carbidopa, caffeine, fluvastatin, ketoprofen, metoprolol, naproxen, propranolol, theophylline, verapamil, diltiazem, levodopa CR, divalproex sodium, digoxin, spironolactone, ibuprofen, neomycin B, captopril, atenolol, caspofingin, clorothiazide, tobramycin, cyclosporin, tacrolimus, and paclitaxel.

Examples of other useful drugs include ulcer treatments such as Carafate™ from Marion Pharmaceuticals, neurotransmitters such as L-DOPA, antihypertensives or saluretics such as Metolazone from Searle Pharmaceuticals, carbonic anhydrase inhibitors such as Acetazolamide from Lederle Pharmaceuticals, insulin like drugs such as glyburide, a blood glucose lowering drug of the sulfonylurea class, synthetic hormones such as Android F from Brown Pharmaceuticals and Testred (methyltestosterone) from ICN Pharmaceuticals, and antiparasitics such as mebendzole (Vermox™, Jansen Pharmaceutical).

Examples of useful proteins include hormones such as insulin, growth hormones including somatomedins, transforming growth factors and other growth factors, antigens for oral vaccines, enzymes such as lactase or lipases, and digestive aids such as pancreatin.

Antigens can be microencapsulated in one or more types of bioadhesive polymer, and subsequently compressed into a tablet or filled into the reservoir of a drug eluting device, to provide a vaccine. The vaccines can be produced to have different retention times in the gastrointestinal tract. The different retention times, among other factors, can stimulate production of more than one type (IgG, IgM, IgA, IgE, etc.) of antibody.

In a preferred method for imaging, a radio-opaque material such as barium sulphate is coated with polymer. Radioactive materials or magnetic materials could be used in place of, or in addition to, radio-opaque materials. Examples of other materials include gases or gas-emitting compounds, which are radioopaque.

Bioadhesive tablets and drug-eluting devices of the invention are especially useful for treatment of inflammatory bowel diseases such as ulcerative colitis and Crohn's disease. In ulcerative colitis, inflammation is restricted to the colon, whereas in Crohn's disease, inflammatory lesions may be found throughout the gastrointestinal tract, from the mouth to the rectum. Sulfasalazine is one of the drugs that is used for treatment of the above diseases. Sulfasalazine is cleaved by bacteria within the colon to sulfapyridine, an antibiotic, and to 5-amino salicylic acid, an anti-inflammatory agent. The 5-amino salicylic acid is the active drug and it is needed locally. Direct administration of the degradation product (5-amino salicylic acid) may be more beneficial. A bioadhesive drug delivery system could improve the therapy by retaining the drug for a prolonged time in the intestinal tract. For Crohn's disease, retention of 5-aminosalicylic acid in the upper intestine is of great importance since bacteria cleave the sulfasalazin in the colon, the only way to treat inflammations in the upper intestine is by local administration of 5-aminosalicylic acid.

The oral dosage formulations described herein can also be used to treat type 2 diabetes, also known as non-insulin-dependent diabetes mellitus (NIDDM). The subject formulations have improved bioavailability over formulations that do not contain the bioadhesive polymers. The formulations of the instant invention are designed to facilitate diffusion of drug into intestinal tissue. In some embodiments, the oral dosage formulations of the invention combine two glucose lowering drugs, such as glipizide and metformin, rosiglitazone and metformin, or pioglitazone and metformin. The combination of two drugs in one formulation functions to improve the different metabolic defects found in type 2 diabetes. For example, glipizide lowers blood glucose primarily by causing more of the body's own insulin to be released, and metformin lowers blood glucose, in part, by helping the body use its own insulin more effectively.

A class of drugs that is particularly suitable for use in the compressed tablets and drug-eluting devices of the invention, particularly the multi-layer tablets of the invention that include a hydrophobic excipient, are hygroscopic and/or deliquescent drugs. The term “hygroscopic” as used herein refers to substances that absorb significant amounts of atmospheric moisture when exposed to conditions of normal ambient relative humidity (RH), for example 10-50% RH. The term “deliquescent” refers to substances that tend to undergo gradual dissolution and/or liquefaction due to attraction and/or absorption of moisture from air when exposed to these conditions. Those skilled in the art will appreciate that over the usual range of ambient temperatures used in drug formulation, hygroscopicity and the state of deliquescence are largely temperature-independent, and that there are varying degrees of hygroscopicity and deliquescence.

Non-limiting examples of hygroscopic and/or deliquescent drugs suitable for use in the present invention include acetylcholine chloride, acetylcamitine, actinobolin, aluminum methionate, aminopentamide, aminopyrine hydrochloride, ammonium bromide, ammonium valerate, amobarbital sodium, anthiolimine, antimony sodium tartrate, antimony sodium thioglycollate, aprobarbital, arginine, aspirin, atropine N-oxide, avoparcin, azithromycin monohydrate, betahistine mesylate, betaine, bethanechol chloride, bismuth subnitrate, bupropion, butamirate, buthalital sodium, butoctamide, cacodylic acid, calcium chloride, calcium glycerophosphate, calcium iodide, carbachol, camitine, caspofungin, ceruletide, chlorophyllin sodium-copper salt, choline alfoscerate, choline salicylate, choline theophyllinate, cilastatin, citicoline, cobalt dichloride, cromolyn disodium, cupric sulfate pentahydrate, cyanocobalamin, cyclobutyrol, cysteine hydrochloride, deaminooxytocin (L-isomer, anhydrous), deanol hemisuccinate, demecarium bromide, dexamethazone phosphate disodium salt, DL-dexpanthenol, dibucaine hydrochloride, dichlorophenarsine hydrochloride, diclofenac sodium, diethylcarbamazine citrate, dimethyl sulfoxidem, drotebanol, echinomycin, ephedrine (anhydrous), ergotamine, ethanolamine, fencamine hydrochloride, ferric chloride, ferrous iodide, ficin, gadobenate dimeglumine, gentamicin C complex sulfate, guanidine, heparin, hexadimethrine bromide, hexamethonium tartrate, hexobarbital sodium, histamine, hydrastine hydrochloride, hyoscyamine hydrobromide, S-[2-[(1-iminoethyl)amino]ethyl]-2-methyl-L-cysteine, imipramine N-oxide, isometheptene hydrochloride, isosorbide, levothyroxine sodium, licheniformins, lobeline sulfate, magnesium chloride hexahydrate, magnesium trisilicate, menadione, mercaptomerin sodium, mersalyl, metaraminol, methacholine chloride, methantheline bromide, methantheline chloride, methitural sodium, L-methyldopa sesquihydrate, methylmethioninesulfonium chloride, mildiomycin, minocycline hydrochloride, mitoxantrone dihydrochloride, morpholine, muscarine chloride, nafronyl acid oxalate, narceine, nicotine, nicotinyl alcohol, nolatrexed dihydrochloride, omeprazole, oryzacidin, oxalic acid, oxophenarsine hydrochloride, panthenol, pantothenic acid (sodium salt), papain, penicillamine hydrochloride, penicillin G (potassium salt), pentamethonium bromide, pentamidine isethionate, pepsin, perazine dihydrochloride, phenobarbital, sodium 5,5-diphenyl hydantoinate, phethenylate sodium, phosphocreatine (calcium salt tetrahydrate), physostigmine sulfate, pilocarpine hydrochloride, pipemidic acid, podophyllotoxin-beta-D-glucoside, potassium carbonate, potassium iodide, pralidoxime mesylate, prednisolone sodium phosphate, procainamide hydrochloride, procaine butyrate, L-proline, promazine hydrochloride, propamidine isethionate, prostacyclin sodium, pyridostigmine bromide, pyronaridine, quinacillin disodium, quinoline, radioactive sodium iodide, reserpilic acid dimethylaminoethyl ester dihydrochloride, secobarbital sodium, silver fluoride, sodium acetate, sodium bromide, sodium propionate, sodium dibunate, sodium dichromate (VI), sodium nitrite, sodium pentosan polysulfate, sodium valproate, soluble sulfamerazine, stibocaptate, streptomycin, succinylcholine bromide, succinylcholine iodide, sulfaquinoxaline, sulisatin disodium, suramin sodium, tamoxifen citrate, taurocholic acid, terazosin hydrochloride, thiobutabarbital sodium, thiopental sodium, ticarcillin disodium, 2,2,2-trichloroethanol, trientine, triethanolamine, triftazin, tolazoline hydrochloride, vinbarbital sodium, viomycin, vitamin B12, zinc iodide, and combinations thereof, and pharmaceutically acceptable hygroscopic and/or deliquescent salts and variants thereof.

The subject formulations can be designed to release drug slowly, quickly or in a step-wise (pulsatile) manner. The formulations may release at least 80% of the drug in 30 minutes, 90 minutes, 4 hours, 8 hours, 12 hours, 16 hours, or up to 24 hours in vitro.

More than one type of drug can be present in a tablet or a drug eluting device of the invention. The drugs can be evenly distributed throughout a medicament or can be heterogeneously distributed in a medicament, such that one drug is fully or partially released before a second drug.

In some embodiments, the drug is incorporated into the bioadhesive layer for delivery to a patient. In other embodiments, it is incorporated into the tablet or drug-eluting device, and a bioadhesive layer is added to at least a part of the exterior of the tablet or drug-eluting device.

The subject dosage formulations will typically comprise an inner core, which comprises one or more drugs, excipients, and/or aborption enhancers that have been compressed to a form a solid, such as a tablet. For example, powdered drug formulations of the invention can be compressed to form a solid, such as a longitudinally compressed tablet. In other embodiments, a drug can be used that in its pure form, under ambient conditions, is a liquid. In some embodiments, the liquid drug that is incorporated into a compressed inner core of the invention is present as a free base or free acid. In embodiments where the drug is a liquid drug (e.g., nicotine, valproic acid), the drug is preferably incorporated into a dosage form of the invention after it has been absorbed onto an absorbent material, such as kaolin clay or Cabosil (colloidal silicon dioxide).

In other embodiments, a solubilized form of an insoluble drug is incorporated into a dosage form of the invention. Solubilized forms of insoluble drugs may be aqueous-based or oil-based. For example, a water-insoluble drug may be dissolved in an organic solvent and then absorbed onto an absorbent material, such as a synthetic aluminosilicate or silicate, which can absorb certain organic solvents while still retaining the properties of a solid.

### Administration of Bioadhesive Materials to Patients

The bioadhesive materials can be administered in a suspension or in an ointment to the mucosal membranes, via the nose, mouth, rectum, or vagina. Pharmaceutically acceptable carriers for oral or topical administration are known and determined based on compatibility with the polymeric material. Other carriers include bulking agents such as METAMUCIL™.

The dosage formulations described herein are suitable for targeted delivery to the small intestine or colon. In embodiments where delivery to the small intestine is desired, the dosage formulations of the invention may be coated with an enteric polymer. In other embodiments, the dosage formulations may be formulated for rectal administration as a bioadhesive suppository.

In some embodiments, the dosage formulation is targeted for delivery to the colonic region of the gastrointestinal tract. It is expected that the extended residence time in the colon with such bioadhesive formulations will result in improved efficacy due to high local drug concentrations and minimum systemic absorption. Targeted delivery to the colon can be achieved, for example, by coating the dosage formulation with a time-dependent and/or pH dependent polymer layer.

For drugs requiring absorption in buccal and sublingual regions of the GI tract, bioadhesive tablets and particularly bioadhesive multiparticulates and nanoparticles are desirable. Drugs absorbed in these sites avoid first-pass metabolism by liver and degradation by GI tract enzymes and harsh pH conditions typically present in the stomach and small intestine. Drugs absorbed in the buccal and sublingual compartments benefit from rapid onset of absorption, typically within minutes of dosing. Particularly suitable are bioadhesive particulates in fast-dissolving dosage forms, e.g., OraSolv (Cima Labs) that disintegrate within 30 sec after dosing and release the bioadhesive particles. Target release profiles include immediate release (IR) and combinations of zero-order controlled release (CR) kinetics and first-order CR kinetics. Preferably, pharmaceutical formulations targeting the buccal and sublingual regions are constructed such that the formulation disintegrates before passing into the esophagus.

For drugs requiring absorption in the stomach and upper small intestine and/or topical delivery to these sites, particularly drugs with narrow absorption windows, bioadhesive, gastroretentive drug delivery systems are the option of choice. Bioadhesive tablets and multiparticulates are formulated to reside for durations greater than 3 hours and optimally greater than 4, 5, or 6 hours in the fed state. Drug release profiles from these systems are tailored to match the gastric residence times, so that greater than 85% of the encapsulated drug is released during the gastric residence time. Target release profiles include zero-order CR kinetics, first-order CR kinetics and combinations of IR and CR kinetics.

For drugs requiring absorption or topical delivery only in the small intestine, enteric-coated, bioadhesive drug delivery systems are a preferred method. Such systems are particularly well suited for topical delivery of therapeutics to Crohn's disease patients. Enteric-coated, bioadhesive tablets and multiparticulates are formulated to reside in the stomach for durations less than 3 hrs in the fed state and less than 1 hr in the fasted state, during which time less than 10% of the encapsulated drug is released, due to the enteric coating. Following gastric emptying, the enteric coating is “triggered” to dissipate, revealing the underlying bioadhesive coating. Suitable triggers include pH and time duration. Typical of enteric polymers utilizing pH as a trigger are Eudragit polymers manufactured by Rohm America: Eudragit L100-55 dissolves at pH values than 5.5, typically found in duodenum; Eudragit L100 dissolves at pH values exceeding 6.0, typically found in jejunum; Eudragit S100 dissolves at pH values exceeding 7.0, typically found in ileum and the ileocecal junction.

Time may be used as a trigger to unmask the bioadhesive coating. Coatings that dissolve after 3 hrs when the dosage form is administered in the fed state and after 1-2 hrs when the dosage form is administered in the fasted state are suitable for bioadhesive delivery systems to the small intestine. Erosion of soluble polymer layers is one means to achieve a time-triggered, enteric dissolution. Polymers such as HPMC, HPC, PVP, PVA or combinations of the above may be used as time-delayed, enteric coatings and timing of the dissolution of the coating can be increased by applying thicker coating weights.

Alternately, non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, triethyl citrate, triacetin, etc., at levels ranging from 0.5 to 50% w/w of the coating and most preferably from 5 to 25% w/w of the coating.

Also suitable are rupturable coating systems, e.g., Pulsincap, that use osmotic forces of swelling from hydrophilic polymers to rupture enteric membranes to reveal underlying bioadhesive coatings.

Target release profiles for the small intestine include: no more than 10% drug release during the first 3 hours post-dosing followed by either IR kinetics, zero-order CR kinetics, first-order CR kinetics and combinations of IR and CR kinetics.

Gastrointestinal Imaging Barium sulphate suspension is the universal contrast medium used for examination of the upper gastrointestinal tract, as described by D. Sutton, Ed., A Textbook of Radiology and Imaging, Vol. 2, Churchill Livingstone, London (1980), even though it has undesirable properties, such as unpalatability and a tendency to precipitate out of solution. Several properties are critical: (a) Particle size: the rate of sedimentation is proportional to particle size (i.e., the finer the particle, the more stable the suspension; (b) Non-ionic medium: charges on the barium sulphate particles influence the rate of aggregation of the particles, aggregation is enhanced in the presence of the gastric contents; (c) Solution pH: suspension stability is best at pH 5.3. However, as the suspension passes through the stomach, it is inevitably acidified and tends to precipitate. The encapsulation of barium sulfate in microspheres of appropriate size provides a good separation of individual contrast elements and may, if the polymer displays bioadhesive properties, help in coating, preferentially, the gastric mucosa in the presence of excessive gastric fluid. With bioadhesiveness targeted to more distal segments of the gastrointestinal tract, it may also provide a kind of wall imaging not easily obtained otherwise.

The double contrast technique, which utilizes both gas and barium sulphate to enhance the imaging process, especially requires a proper coating of the mucosal surface. Air or carbon dioxide must be introduced to achieve a double contrast. This is typically achieved via a nasogastric tube to provoke a controlled degree of gastric distension. Studies indicate that comparable results may be obtained by the release of individual gas bubbles in a large number of individual adhesive microspheres and that this imaging process may be used to image intestinal segments beyond the stomach.

### Methods of Making Bioadhesive Rate-Controlled Oral Dosages

As described above, one method of making dosage formulations according to the invention comprises inserting a core into an extruded bioadhesive polymer tube. The extruded bioadhesive polymer tube may comprise Spheromer™ I (p(FASA), as described in U.S. Pat. No. 5,955,096 to Mathiowitz et al.), Spheromer™ II (oligomers and metal oxides, as described in U.S. Pat. No. 5,985,312 to Jacob et al.), Spheromer™ III (DOPA side chains grafted onto a non-biodegradable polymeric backbone, see U.S. application Ser. No. 11/009,327, filed Dec. 9, 2004, and WO 2005/056708), or other bioadhesive polymers available commercially along with suitable plasticizers, pore-forming agents, and solvents. Other thermoplastic polymers can be added to modify the moldability and mechanical strength of the bioadhesive polymer tube.

In a preferred embodiment of the invention, the extruded polymer tube is prepared via a hot-melt extrusion process, where the desired bioadhesive polymer is fed into the extruder as a pellet, flake, powder, etc. along with plasticizer. In certain such embodiments, the materials are blended as they are propelled continuously along a screw through regions of high temperature and pressure to form the polymer extrudate. The extrudate may then be pushed from the extruder through a die having the desired shape and dimension to form a tube, e.g., a cylindrical tube. The tube may be cooled after extrusion. The dimensions of the tube can be varied to accommodate the inner core system. The inner diameter of the tube can be configured to conform to the desired circumferential dimension of the preformed, pre-pressed inner system containing the therapeutic agent (s). The thickness of the tube is determined in part by the polymer/plasticizer type as well its behavior with respect to the external fluid.

The bioadhesive nature of the polymer tube may also be controlled by utilizing different types of polymers and excipients. Inorganic metal oxides may be added to improve adhesion. Pore formers may also be added to control porosity. Entero-soluble polymers may be added to promote disintegration of the bioadhesive layer. For example, after drug release, the bioadhesive layer may become weak and disintegrate upon release of entero-soluble polymers from the layer. Drugs may also be added into the polymer layer either as a plasticizer or pore-forming agent. Once formed, the inner system, preferably in the form of a longitudinally compressed tablet, is inserted into the tube, and the two components are fused together to get a finished dosage form.

Methods for Production of the Hollow Bioadhesive Cylinder

Prior to hot-melt extrusion of the hollow cylinder, a polyanhydride polymer e.g., poly(fumaric-co-sebacic) acid or polyadipic acid and 20% triethyl citrate (based on polymer weight) are mixed in a planetary mixer. Extrusion can be performed using either a MP 19 TC25 laboratory scale co-rotating twin screw extruded of APV Baker (Newcastle-under-Lyme, UK) or a Killian extruder (Killian extruder Inc., Cedar Grove, N.J.). Both machines are equipped with a standard screw profile with two mixing sections, an annual die with metal insert for the production of the cylinder and twin screw powder feeder. Typical extrusion conditions are: a screw speed of 5 rpm, a powder feed rate of 0.14 kg/hr and a temperature profile of 125-115-105-80-65° C. from the powder feeder towards the die. The cylinders (internal diameter of 7 mm and wall thickness of 1 mm) are cut into 1 cm long cylinders.

Methods for Production of the Inner Core System

Longitudinally compressed core tablets containing the therapeutic agent and other components may be compressed onto a single or multilayer tableting machine equipped with deep fill or regular tooling. For example, the therapeutic agent either alone or in combination with a rate controlling polymer and other excipients is mixed by stirring, ball milling, roll milling or calendaring and pressed into a solid having dimensions conforming to an internal compartment defined by the extruded polymer cylinder. The inner core system may be a pre-fabricated osmotic system that is inserted into the bioadhesive cylinder with orifices aligned along the open ends of the cylinder.

One or more layers containing different therapeutic agents can be included in a multilayer tablet. For example, in certain embodiments, the invention relates to multi-layer tablets comprising a first, a second and a third layer, where each layer includes one or more drugs and one or more excipients, where the first layer forms the core of the tablet, the second layer is adjacent to one side of the first layer, and the third layer is adjacent to the opposite side of the first layer. At least one layer of the tablet includes a hydrophobic excipient and at least one drug in the tablet is hygroscopic, deliquescent or both. Preferably, at least one hygroscopic and/or deliquescent drug and at least one hydrophobic excipient are present (e.g., blended together) in at least one layer of a tablet.

Exemplary hydrophobic excipients include celluloses, particularly methylcellulose and ethylcellulose.

Such tablets optionally include one excipient present in an amount sufficient to be at least partially rate-controlling with respect to release of the drug from the tablet. Typically, tablets that include a rate-controlling excipient (e.g., a rate-controlling polymer) contain about 30% to about 60% by weight of the rate-controlling excipient. Alternatively, the amount of rate-controlling excipient is selected relative to the amount of drug in the tablet. In such cases, the weight of the rate-controlling excipient is about two times to about five times, such as about two times to about three times greater than the weight of the drug.

Typically, the inner and outer layers contain different proportions of each component (including the drug(s)), thereby establishing a gradient-type composition. In an exemplary embodiment, the first (inner) layer contains the greatest weight percentage of the drug(s). Accordingly, the second and third layers and any additional layers present contain lesser amounts of drug. In multi-layer tablets having more than three layers (e.g., those having a fourth and optionally a fifth layer), the additional layers can, for example, contain no drug or contain successively lesser amounts of drug. In general, layers the same distance away from the first or inner layer will contain approximately equal amount of drug, such that the tablet is essentially symmetrical about the inner layer. For tablets containing two or more drugs, the drugs can both be present in one or more layers or the different drugs are present in separate layers (i.e., the drugs are not mixed together in one layer).

In certain aspects, multi-layer compressed tablets of the invention exhibit an approximately zero-order release of drug in in vitro testing and/or in vivo administration. For formulations where delivery to the stomach is desired, zero-order release advantageously occurs over about 6-12 hours, particularly 8-10 hours. For formulations where delivery to the stomach and small intestine are desired, zero-order release advantageously occurs over about 8-16 hours, particularly 10-14 hours. For formulations where delivery to the small intestine and colon are desired, zero-order release advantageously occurs over about 16-30 hours, particularly 22-26 hours.

Multi-layer or gradient tablets can be assembled in several different ways. In one embodiment, the tablet comprises at least one solid inner layer and two solid outer layers, each comprising one or more drugs and one or more pharmaceutical polymers and/or pharmaceutical excipients. In order to produce a gradient effect, the amount of drug and/or excipient differs among the inner and outer layers. For example, the one or more inner layers can comprise at least 34% of the total amount of the drug in the tablet and one or more polymer(s) and/or excipients(s), and each of the two outer layers can comprise not more than 33% of the total amount of drug in the tablet and one or more polymer(s) and/or excipients(s). Such tablets can also be used to commence release of different drugs at different times, by inclusion of different drugs in separate layers.

The core may contain, in any desired order, discrete layers of different composition. For example, different layers may contain the same or different drugs with varying release profiles. For example, one layer may comprise a drug formulated for extended release, and in a second layer, the same or different drug may be formulated for immediate release (or two layers may have different profiles of controlled release of the same or different drugs). Similarly, different drugs may be present in different layers, with the formulation of each layer adjusted to account for characteristics of the particular drug in that layer. For example, multi-layer tablets can be designed to incorporate an insoluble drug in one layer, optionally with permeation and/or dissolution enhancers, and a highly soluble drug in another layer. The subject multi-layer formulations may also have two or more layers of the same or different drugs for release at the same or varying target sites of absorption. For example, one layer may comprise a drug for release in the stomach and another layer (e.g., coated with a further enteric coating) may comprise a drug targeted for release in the small intestine.

In another embodiment, the multi-layer tablet consists of a solid inner layer and two solid outer layers, each comprising a drug and one or more pharmaceutical polymers or pharmaceutical excipients, wherein at least one polymer or excipient is hydrophobic. Tablets of this embodiment preferably provide approximately zero-order or linear release kinetics. In still another embodiment, the multi-layer tablet is enteric coated.

One or more layers of the tablet can contain permeation enhancers and/or dissolution enhancers to provide permeability enhancement of drugs through mucosal lining of the gastrointestinal tract (GIT). An absorption enhancer facilitates the uptake of a drug across the gastrointestinal epithelium. Absorption enhancers include compounds that improve the ability of a drug to be solubilized in the aqueous environment in which it is originally released and/or in the lipophilic environment of the mucous layer lining of the intestinal walls. Absorption enhancers further include compounds that increase disorder of the hydrophobic region of the membrane exterior of intestinal cells, promote leaching of membrane proteins that results in increased transcellular transport, or widen the pore radius between cells for increased paracellular transport. Examples of absorption enhancers include sodium caprate, ethylenediamine tetra(acetic acid) (EDTA), citric acid, lauroylcarnitine, palmitoylcarnitine, tartaric acid and other agents known to increase GI permeability. Other suitable absorption enhancers include sodium salicylate, sodium 5-methoxysalicylate, indomethacin, diclofenac, polyoxyethylene ethers, sodium laurylsulfate, quaternary ammonium compounds, sodium deoxycholate, sodium cholate, octanoic acid, decanoic acid, glyceryl-1-monooctanoate, glyceryl-1-monodecanoate, DL-phenylalanine ethylacetoacetate enamine, chlorpromazine, D-myristoyl-L-propyl-L-prolyl-glycinate, concanavaline A, DL-á-glycerophosphate, and 3-amino-1-hydroxypropylidene-1,1-diphosphonate.

Alternatively, or in addition, the tablet is coated to provide additional control over diffusion of the drug or exposure of the tablet to the gastrointestinal tract (e.g., with an enteric coating). The diffusion-limiting coating can be a pharmaceutically-accepted polymeric coating material, such as methylmethacrylates (Eudragits™, Rohm and Hass; Kollicoat™, BASF), zein, cellulose acetate, cellulose phthalate and hydroxypropylmethylcellullose. The coatings can be applied using a variety of techniques including fluidized-bed coating, pan-coating and dip-coating.

Separately or in combination with the bioadhesive coating, a bioadhesive (such as those described above) can be included in one or more layers of a compressed inner core of the invention.

Multi-layer tablets of the invention are readily prepared. In one example, the drug(s) is/are mixed with a compressible sugar and granulated with a binder solution of compressible sugar in purified water. Subsequent to drying, the granules are mixed with different amounts of colloidal silicon dioxide (Cabosil™) and magnesium stearate. The granules are mixed in different proportions with stearic acid or monosterate (30, 50, 70%, for example) and then fed into a multilayer tableting machine (such as a Korsch or Fette tableting machine) to yield a trilayer tablet. Additional layers, often with varying amount of drug granules (e.g., greater drug concentration in the center layer and decreasing in each subsequent outer layer), can readily be added. In certain embodiments, the outermost layers do not include a drug.

In some embodiments, the compressed inner core is a tablet that is coated, either entirely or partially, with a gelatin, which is further coated with a bioadhesive polymer on at least one of its surfaces. In some embodiments, the gelatin layer itself may comprise a bioadhesive polymer.

Gelatin layers can be applied to a subject compressed inner core by an enrobing technique. For example, in some embodiments, a pre-compressed tablet is fully enrobed in a soft elastic film material, such as a gelatin film, in a relatively dry state and at a relatively low temperature. It is fully enrobed between two layers of the applied elastic film material of selected thickness and composition, which layers substantially conform to the contours of the pre-compressed tablet and which are sealed to each other along a single line encircling the pre-compressed tablet and lying substantially in a common plane. The film layers, when applied to the pre-compressed tablet, exhibit substantially low water activity and have an elastic plastic character. Typically, the film material applied in tablet-enrobing manner to the tablet is a gelatin-base film so formulated that, as applied to and sealed around the tablet, it conforms tightly to the tablet contours and bonds securely to the tablet surfaces and dries to a hard state. Various enrobing methods are described in U.S. Pat. No. 6,482,516 and U.S. Patent Application Nos. 20040161527, 20030215563, and 20030059614, each of which is incorporated herein by reference.

Typically, the hardness of a compressed tablet of the invention is between 2 kp-30 kp. In some embodiments, it is between 2 kp-5 kp, 2 kp-10 kp, 2 kp-15 kp, 2 kp-20 kp, or 2 kp-25 kp. In yet other embodiments, it is between 5 kp-15 kp, 10 kp-20 kp, or 15 kp-30 kp.

Tablets and drug eluting devices of the invention typically weigh at least 5 mg. Tablets and drug eluting devices can also weigh at least 10 mg, at least 15 mg, at least 25 mg, at least 50 mg, at least 100 mg, at least 500 mg or at least 1000 mg. Typically, such objects weigh 10 mg to 500 mg.

Tablets and drug eluting devices of the invention typically measure at least 2 mm in one dimension. For example, tablets and drug eluting devices can measure at least 5 mm, at least 10 mm, at least 15 mm or at least 20 mm in one dimension. Typically, the diameter of the tablets and drug eluting devices is 2 to 40 mm, preferably 10 to 30 mm such as 20 to 26 mm. Mini-tablets have a diameter of 2 mm to about 5 mm. Such tablets and devices can measure at least 2 mm, at least 5 mm, at least 10 mm, at least 15 mm or least 20 mm in a second dimension and, optionally, a third dimension. Preferably, the tablet or drug eluting device is of a size that facilitates swallowing by a subject.

The volume of a typical tablet or drug eluting device of the invention is at least 0.008 mL, at least 0.01 mL, at least 0.05 mL, at least 0.1 mL, at least 0.125 mL, at least 0.2 mL, at least 0.3 mL, at least 0.4 mL or at least 0.5 mL, such as from 0.008 mL to 0.5 mL.

Methods of Insertion of the Inner Core System into the Bioadhesive Cylinder

The preformed inner core with a diameter slightly smaller than the inner diameter of the tube may be either manually or mechanically inserted into the tube and heated to fuse the two units. Alternately, the core may be inserted into the tube by a positive placement core insertion mechanism on the tableting machine. Initially, the extruded tube may be placed into the die of the machine followed by insertion of the compressed core into the internal compartment of the tube, and the two components compressed to get the finished dosage form. Alternatively, the dosage form may be prepared via simultaneous extrusion of the bioadhesive tube and expandable inner composition using an extruder capable of such an operation.

In certain embodiments, the bioadhesive polymer is applied to the tablet by compression coating. In some embodiments, compression coating comprises adding one-half of the bioadhesive polymeric layer to a die, positioning the compressed tablet in a flat position in the die, adding the remaining half of the bioadhesive polymeric layer to the die, and compressing the bioadhesive polymeric layers and tablet together.

In other embodiments, compression coating comprises adding the bioadhesive polymeric layer to a die, loosely compressing the bioadhesive polymeric layer (e.g., with an upper shaped punch that creates room for insertion of the longitudinally compressed die), and inserting the tablet. Preferably, the upper punch has a telescopic core rod within it. The telescopic rod will displace the coating material and will compact it at the bottom and sides of the die. Once a space has been created, the pre-compressed inner core is inserted. Creating room for the inner core and insertion of the inner core can be done sequentially or in the same process. In certain embodiments, the pre-compressed inner core is itself used as a telescopic core rod.

In other embodiments, the pre-compressed inner core is fully coated with a bioadhesive coating. One or both ends of the coated tablet are then circumferentially perforated by a laser. In such embodiments, it is expected that when the dosage formulation takes up water, the perforated part of the dosage form will open up as a flap, thereby enabling release of the drug.

### Bioadhesives

As generally used herein “bioadhesives” or “bioadhesive materials” refer to polymers which have or are modified to have improved bioadhesion.

As used herein “bioadhesion” generally refers to the ability of a material to adhere to a biological surface for an extended period of time. Bioadhesion requires a contact between the bioadhesive material and the receptor surface, the bioadhesive material penetrates into the crevice of the surface (e.g., tissue and/or mucus) and chemical bonds form. Thus the amount of bioadhesive force is affected by both the nature of the bioadhesive material, such as a polymer, and the nature of the surrounding medium. Bioadhesive forces are measured in units of N/m², by methods defined in U.S. Pat. No. 6,197,346, which is herein incorporated by reference.

### Polymers

Suitable bioadhesive polymeric coatings are disclosed in U.S. Pat. Nos. 6,197,346, 6,217,908 and 6,365,187, the contents of which are incorporated herein by reference, and include soluble and insoluble, biodegradable and nonbiodegradable polymers. These can be hydrogels or thermoplastics, homopolymers, copolymers or blends, and/or natural or synthetic polymers. The preferred polymers are synthetic polymers, with controlled synthesis and degradation characteristics. Particularly preferred polymers are anhydride copolymers of fumaric acid and sebacic acid (P(FA:SA)), which have exceptionally good bioadhesive properties when administered to the gastrointestinal tract. Examples of P(FA:SA) copolymers include those having a 1:99 to 99:1 ratio of fumaric acid to sebacic acid, such as 5:95 to 75:25, for example, 10:90 to 60:40 or at least 15:85 to 25:75. Specific examples of such copolymers have a 20:80 or a 50:50 ratio of fumaric acid to sebacic acid.

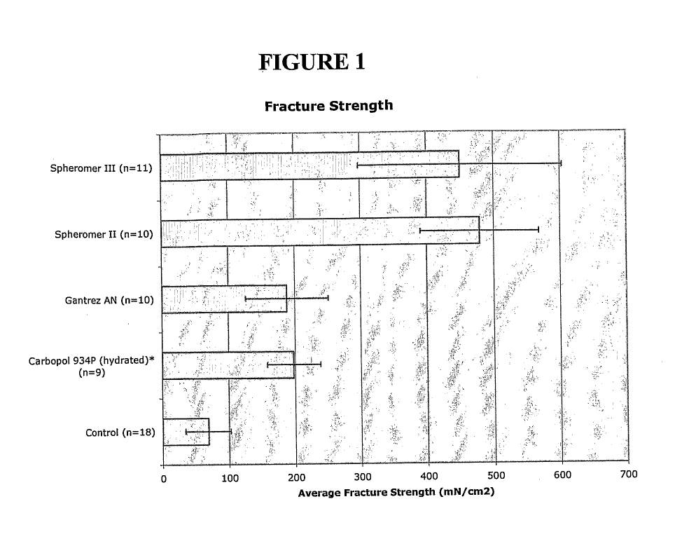

Polymers used in tablets and drug eluting devices of the invention preferably produce a bioadhesive interaction (fracture strength) of at least 100 N/m² (10 mN/cm²) when applied to the mucosal surface of rat intestine. The fracture strength of the tablets and drug eluting devices is advantageously at least 250 N/m², at least 500 N/m² or at least 1000 N/m². For example, the fracture strength of a polymer-containing tablet or drug eluting device can be from 100 to 500 N/m². The forces described herein refer to measurements made upon rat intestinal mucosa, unless otherwise stated. The same adhesive measurements made on a different species of animal will differ from those obtained using rats. This difference is attributed to both compositional and geometrical variations in the mucous layers of different animal species as well as cellular variations in the mucosal epithelium. However, the data shows that the same general trends prevail no matter what animal is studied (i.e., p(FA:SA) produces stronger adhesions than polylactic acid (PLA) in rats, sheep, pigs, etc.). See, for example, Example I and the results presented in FIG. 1. For example, the fracture strength of tablets and drug eluting devices of the invention on rat intestine is generally at least 125 N/m², such as at least 150 N/m², at least 250 N/m², at least 500 N/m² or at least 1000 N/m². For example, the fracture strength of tablets and drug eluting devices of the invention on pig intestine is generally at least 125 N/m², such as at least 150 N/m², at least 250 N/m², at least 500 N/m² or at least 1000 N/m².

The fracture strength of a tablet or drug eluting device can be measured according to the methods disclosed by Duchene et al. in Drug Dev. Ind. Pharm., 14:283-318 (1988). Briefly, the tablet is attached on one side to a tensile tester and is contacted with a testing surface (e.g., a mucosal membrane) on the opposite surface. The tensile tester measures the force required to displace the tablet or drug eluting device from the testing surface. Common tensile testers include a Texture Analyzer and the Instron tensile tester.

In the preferred method for mucoadhesive testing, tablets are pressed using flat-faced tooling, 0.3750″ (9.525 mm) in diameter. Tablet weight will depend on composition; in most cases, the tablets have a final weight of 200 mg. These tablets are then glued to a plastic 10 mm diameter probe using a common, fast-drying cyanoacrylate adhesive. Once the tablets are firmly adhered to the probe, the probe is attached to the Texture Analyzer. The Texture Analyzer is fitted with a 1 kg load cell for maximum sensitivity. The following settings are used:

| | | | |
|---|---|---|---|
| Pre-Test Speed | 0.4 mm/sec | Stop Plot At | Final Position |
| Test Speed | 0.1 mm/sec | Tare Mode | Auto |
| Post-Test Speed | 0.1 mm/sec | Delay Acquisition | Off |
| Applied Force | 20.0 g | Advanced Options | On |
| Return Distance | 0 mm | Proportional Gain | 0 |
| Contact Time | 420 s | Integral Gain | 0 |
| Trigger Type | Auto | Differential Gain | 0 |
| Trigger Force | 0.5 g | Max. Tracking | 0 mm/sec |
| | | Speed | |

The Test and Post-Test Speeds are as low as the instrument will allow, to ensure a maximum number of data points captured. The Pre-Test speed is used only until the probe encounters the Trigger Force; i.e., prior to contacting the tissue.

The Proportional, Integral, and Differential Gain are set to 0. These settings, when optimized, maintain the system at the Applied Force for the duration of the Contact Time. With soft tissue as a substrate, however, the probe and tablet are constantly driven into the deformable surface. This results in visible damage to the tissue. Thus, the probe and tablet are allowed to relax gradually from the Applied Force by setting these parameters to 0. The tracking speed, which is a measure of how rapidly the feedback is adjusted, is also set to 0.

The tissue on which the tablets are tested is secured in the Mucoadhesive Rig; the rig is then completely immersed in a 600 mL Pyrex beaker containing 375 mL of PBS. The tissue is maintained at approximately 37° C. for the duration of the test; no stirring is used as the machine can detect the oscillations from the stir bar.

In the past, two classes of polymers have shown useful bioadhesive properties, hydrophilic polymers and hydrogels. In the large class of hydrophilic polymers, those containing carboxylic groups (e.g., poly[acrylic acid]) exhibit the best bioadhesive properties. It is thus expected that polymers with the highest concentrations of carboxylic groups are preferred materials for bioadhesion on soft tissues. In other studies, the most promising polymers were sodium alginate, carboxymethylcellulose, hydroxymethylcellulose and methylcellulose. Some of these materials are water-soluble, while others are hydrogels.

Rapidly bioerodible polymers such as poly[lactide-co-glycolide], polyanhydrides, and polyorthoesters, whose carboxylic groups are exposed on the external surface as their smooth surface erodes, are particularly suitable for bioadhesive drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers suitable for the present invention include proteins (e.g., hydrophilic proteins), such as zein, modified zein, casein, gelatin, gluten, serum albumin, or collagen, and polysaccharides such as cellulose, dextrans, polyhyaluronic acid, polymers of acrylic and methacrylic esters and alginic acid. These are generally less suitable for use in bioadhesive coatings due to higher levels of variability in the characteristics of the final products, as well as in degradation following administration. Synthetically modified natural polymers include alkyl celluloses, hydroxyalkyl celluloses, cellulose ethers, cellulose esters, and nitrocelluloses.

Representative synthetic polymers for use in bioadhesive coatings include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other polymers suitable for use in the invention include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, and polyvinylphenol. Representative bioerodible polymers for use in bioadhesive coatings include polylactides, polyglycolides and copolymers thereof, poly(ethylene terephthalate), poly(butic acid), poly(valeric acid), poly(lactide-co-caprolactone), poly[lactide-co-glycolide], polyanhydrides (e.g., poly(adipic anhydride)), polyorthoesters, blends and copolymers thereof.

Polyanhydrides are particularly suitable for use in bioadhesive delivery systems because, as hydrolysis proceeds, causing surface erosion, more and more carboxylic groups are exposed to the external surface. However, polylactides erode more slowly by bulk erosion, which is advantageous in applications where it is desirable to retain the bioadhesive coating for longer durations. In designing bioadhesive polymeric systems based on polylactides, polymers that have high concentrations of carboxylic acid are preferred. The high concentrations of carboxylic acids can be attained by using low molecular weight polymers (MW of 2000 or less), because low molecular weight polymers contain a high concentration of carboxylic acids at the end groups.

The polymers listed above can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif., or can alternatively be synthesized from monomers obtained from these suppliers using standard techniques.

When the bioadhesive polymeric coating is a synthetic polymer coating, the synthetic polymer is typically selected from polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers of thereof. In an exemplary embodiment, the synthetic polymer is poly(fumaric-co-sebacic) anhydride.

Another group of polymers suitable for use as bioadhesive polymeric coatings are polymers having a hydrophobic backbone with at least one hydrophobic group pendant from the backbone. Suitable hydrophobic groups are groups that are generally non-polar. Examples of such hydrophobic groups include alkyl, alkenyl and alkynyl groups. Preferably, the hydrophobic groups are selected to not interfere and instead to enhance the bioadhesiveness of the polymers.

A further group of polymers suitable for use as bioadhesive polymeric coatings are polymers having a hydrophobic backbone with at least one hydrophilic group pendant from the backbone. Suitable hydrophilic groups include groups that are capable of hydrogen bonding or electrostatically bonding to another functional group. Example of such hydrophilic groups include negatively charged groups such as carboxylic acids, sulfonic acids and phosphonic acids, positively charged groups such as (protonated) amines and neutral, polar groups such as amides and imines. Preferably, the hydrophilic groups are selected to not interfere and instead to enhance the bioadhesiveness of the polymers. The hydrophilic groups can be either directly attached to a hydrophobic polymer backbone or attached through a spacer group. Typically, a spacer group is an alkylene group, particularly a C₁-C₈ alkyl group such as a C₂-C₆ alkyl group. Preferred compounds containing one or more hydrophilic groups include amino acids (e.g., phenyalanine, tyrosine and derivatives thereof) and amine-containing carbohydrates (sugars) such as glucosamine.

Polymers can be modified by increasing the number of carboxylic groups accessible during biodegradation, or on the polymer surface. The polymers can also be modified by binding amino groups to the polymer. The polymers can be modified using any of a number of different coupling chemistries available in the art to covalently attach ligand molecules with bioadhesive properties to the surface-exposed molecules of the polymeric microspheres.

The attachment of any positively charged ligand, such as polyethyleneimine or polylysine, to a polymer may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most polymers with the appropriate chemistry, such as with carbodiimidazole (CDI), and be expected to influence the binding to the gut. For example, polyclonal antibodies raised against components of mucin or else intact mucin, when covalently coupled to a polymer, would provide for increased bioadhesion. Similarly, antibodies directed against specific cell surface receptors exposed on the lumenal surface of the intestinal tract would increase the residence time when coupled to polymers using the appropriate chemistry. The ligand affinity need not be based only on electrostatic charge, but other useful physical parameters such as solubility in mucin or specific affinity to carbohydrate groups.

The covalent attachment of any of the natural components of mucin in either pure or partially purified form to the polymers generally increases the solubility of the polymer in the mucin layer. The list of useful ligands include but are not limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, such as polyaspartic acid and polyglutamic acid, may also increase bioadhesiveness. The polyamino chains would increase bioadhesion by means of chain entanglement in mucin strands as well as by increased carboxylic charge.

Polymer-Metal Complexes

As disclosed in U.S. Pat. Nos. 5,985,312, 6,123,965 and 6,368,586, the contents of which are incorporated herein by reference, polymers, such as those named above, having a metal compound incorporated therein have a further improved ability to adhere to tissue surfaces, such as mucosal membranes, and are suitable for use in the invention. The metal compound incorporated into the polymer can be, for example, a water-insoluble metal oxide. The incorporation of metal compounds into a wide range of different polymers, even those that are not normally bioadhesive, often improves their ability to adhere to tissue surfaces such as mucosal membranes.

Metal compounds that can be incorporated into polymers to improve their bioadhesive properties preferably are water-insoluble metal compounds, such as water-insoluble metal oxides and metal hydroxides, which are capable of becoming incorporated into and associated with a polymer to improve the bioadhesiveness of the polymer. As defined herein, a water-insoluble metal compound is defined as a metal compound with little or no solubility in water, for example, less than about 0.0 to 0.9 mg/ml.

The water-insoluble metal compounds can be derived from a wide variety of metals, including, but not limited to, calcium, iron, copper, zinc, cadmium, zirconium and titanium. The water insoluble metal compound preferably is a metal oxide or hydroxide. Water insoluble metal compounds of multivalent metals are preferred. Representative metal oxides suitable for use in the compositions described herein include cobalt (I) oxide (CoO), cobalt (III) oxide (CO₂O₃), selenium oxide (SeO₂), chromium (IV) oxide (CrO₂), manganese oxide (MnO₂), titanium oxide (TiO₂), lanthanum oxide (La₂O₃), zirconium oxide (ZrO₂), silicon oxide (SiO₂), scandium oxide (Sc₂O₃), beryllium oxide (BeO), tantalum oxide (Ta₂O₅), cerium oxide (CeO₂), neodymium oxide (Nd₂O₃), vanadium oxide (V₂O₅), molybdenum oxide (Mo₂O₃), tungsten oxide (WO), tungsten trioxide (WO₃), samarium oxide (Sm₂O₃), europium oxide (Eu₂O₃), gadolinium oxide (Gd₂O₃), terbium oxide (Tb₄O₇), dysprosium oxide (Dy₂O₃), holmium oxide (Ho₂O₃), erbium oxide (Er₂O₃), thulium oxide (Tm₂O₃), ytterbium oxide (Yb2O₃), lutetium oxide (Lu₂O₃), aluminum oxide (Al₂O₃), indium oxide (InO₃), germanium oxide (GeO₂), antimony oxide (Sb₂O₃), tellurium oxide (TeO₂), nickel oxide (NiO), and zinc oxide (ZnO). Other oxides include barium oxide (BaO), calcium oxide (CaO), nickel (III) oxide (Ni₂O₃), magnesium oxide (MgO), iron (II) oxide (FeO), iron (III) oxide (Fe₂O₃), copper (II) oxide (CuO), cadmium oxide (CdO), and zirconium oxide (ZrO₂).

Preferred characteristics of the metal compound include: (a) substantial insolubility in aqueous environments, such as acidic or basic aqueous environments (such as those present in the gastric lumen); and (b) ionizable surface charge at the pH of the aqueous environment.

The water-insoluble metal compounds can be incorporated into the polymer by one of the following mechanisms: (a) physical mixtures which result in entrapment of the metal compound; (b) ionic interaction between metal compound and polymer; (c) surface modification of the polymers which would result in exposed metal compound on the surface; and (d) coating techniques such as fluidized bed, pan coating, or any similar methods known to those skilled in the art, which produce a metal compound enriched layer on the surface of the device. In one embodiment, nanoparticles or microparticles of the water-insoluble metal compound are incorporated into the polymer.

In one embodiment, the metal compound is provided as a fine particulate dispersion of a water-insoluble metal oxide which is incorporated throughout the polymer or at least on the surface of the polymer which is to be adhered to a tissue surface. The metal compound also can be incorporated in an inner layer of the polymer and exposed only after degradation or else dissolution of a “protective” outer layer. For example, a tablet core containing a polymer and metal may be covered with an enteric coating designed to dissolve when exposed to gastric fluid. The metal compound-enriched core then is exposed and become available for binding to GI mucosa.

Fine metal oxide particles can be produced for example by micronizing a metal oxide by mortar and pestle treatment to produce particles ranging in size, for example, from 10.0 to 300 nm. The metal oxide particles can be incorporated into the polymer, for example, by dissolving or dispersing the particles into a solution or dispersion of the polymer.

Advantageously, metal compounds which are incorporated into polymers to improve their bioadhesive properties can be metal compounds which are already approved by the FDA as either food or pharmaceutical additives, such as zinc oxide.

Suitable polymers which can be used and into which the metal compounds can be incorporated include soluble and water-insoluble, and biodegradable and nonbiodegradable polymers, including hydrogels, thermoplastics, and homopolymers, copolymers and blends of natural and synthetic polymers, provided that they have the requisite fracture strength when mixed with a metal compound. In additional to those listed above, representative polymers which can be used in conjunction with a metal compound include hydrophilic polymers, such as those containing carboxylic groups, including polyacrylic acid. Bioerodible polymers including polyanhydrides, poly(hydroxy acids) and polyesters, as well as blends and copolymers thereof also can be used. Representative bioerodible poly(hydroxy acids) and copolymers thereof which can be used include poly(lactic acid), poly(glycolic acid), poly(hydroxy-butyric acid), poly(hydroxyvaleric acid), poly(caprolactone), poly(lactide-co-caprolactone), and poly(lactide-co-glycolide). Polymers containing labile bonds, such as polyanhydrides and polyorthoesters, can be used optionally in a modified form with reduced hydrolytic reactivity. Positively charged hydrogels, such as chitosan, and thermoplastic polymers, such as polystyrene also can be used.

Representative natural polymers which also can be used include proteins, such as zein, modified zein, casein, gelatin, gluten, serum albumin, or collagen, and polysaccharides such as dextrans, polyhyaluronic acid and alginic acid. Representative synthetic polymers include polyphosphazenes, polyamides, polycarbonates, polyacrylamides, polysiloxanes, polyurethanes and copolymers thereof. Celluloses also can be used. As defined herein the term “celluloses” includes naturally occurring and synthetic celluloses, such as alkyl celluloses, cellulose ethers, cellulose esters, hydroxyalkyl celluloses and nitrocelluloses. Exemplary celluloses include ethyl cellulose, methyl cellulose, carboxymethyl cellulose, hydroxymethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, cellulose triacetate and cellulose sulfate sodium salt.

Polymers of acrylic and methacrylic acids or esters and copolymers thereof can be used. Representative polymers which can be used include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other polymers which can be used include polyalkylenes such as polyethylene and polypropylene; polyarylalkylenes such as polystyrene; poly(alkylene glycols), such as poly(ethylene glycol); poly(alkylene oxides), such as poly(ethylene oxide); and poly(alkylene terephthalates), such as poly(ethylene terephthalate). Additionally, polyvinyl polymers can be used, which, as defined herein includes polyvinyl alcohols, polyvinyl ethers, polyvinyl esters and polyvinyl halides. Exemplary polyvinyl polymers include poly(vinyl acetate), polyvinyl phenol and polyvinylpyrrolidone.

Water soluble polymers can also be used. Representative examples of suitable water soluble polymers include polyvinyl alcohol, polyvinylpyrrolidone, methyl cellulose, hydroxypropyl cellulose, hydroxypropylmethyl cellulose and polyethylene glycol, copolymers of acrylic and methacrylic acid esters, and mixtures thereof. Water insoluble polymers also can be used. Representative examples of suitable water insoluble polymers include ethylcellulose, cellulose acetate, cellulose propionate (lower, medium or -higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene oxide), poly(ethylene terephthalate), poly(vinyl isobutyl ether), poly(vinyl acetate), poly(vinyl chloride), polyurethanes, and mixtures thereof. In one embodiment, a water insoluble polymer and a water soluble polymer are used together, such as in a mixture. Such mixtures are useful in controlled drug release formulations, wherein the release rate can be controlled by varying the ratio of water soluble polymer to water insoluble polymer.

Polymers varying in viscosity as a function of temperature or shear or other physical forces also may be used. Poly(oxyalkylene) polymers and copolymers such as poly(ethylene oxide)-poly(propylene oxide) (PEO-PPO) or poly(ethylene oxide)-poly(butylene oxide) (PEO-PBO) copolymers, and copolymers and blends of these polymers with polymers such as poly(alpha-hydroxy acids), including but not limited to lactic, glycolic and hydroxybutyric acids, polycaprolactones, and polyvalerolactones, can be synthesized or commercially obtained. For example, polyoxyalkylene copolymers are described in U.S. Pat. Nos. 3,829,506, 3,535,307, 3,036,118, 2,979,578, 2,677,700 and 2,675,619. Polyoxyalkylene copolymers are sold, for example, by BASF under the tradename PLURONICS™. These materials are applied as viscous solutions at room temperature or lower which solidify at the higher body temperature. Other materials with this behavior are known in the art, and can be utilized as described herein. These include KLUCEL™ (hydroxypropyl cellulose), and purified konjac glucomannan gum.

Other suitable polymers are polymeric lacquer substances based on acrylates and/or methacrylates, commonly called EUDRAGIT™ polymers (sold by Rohm America, Inc.). Specific EUDRAGIT™ polymers can be selected having various permeability and water solubility, which properties can be pH dependent or pH independent. For example, EUDRAGIT™ RL and EUDRAGIT™ RS are acrylic resins comprising copolymers of acrylic and methacrylic acid esters with a low content of quaternary ammonium groups, which are present as salts and give rise to the permeability of the lacquer films, whereas EUDRAGIT™ RL is freely permeable and EUDRAGIT™ RS is slightly permeable, independent of pH. In contrast, the permeability of EUDRAGIT™ L is pH dependent. EUDRAGIT™ L is an anionic polymer synthesized from methacrylic acid and methacrylic acid methyl ester. It is insoluble in acids and pure water, but becomes increasingly soluble in a neutral to weakly alkaline solution by forming salts with alkalis. Above pH 5.0, the polymer becomes increasingly permeable.

Polymer solutions that are liquid at an elevated temperature but solid or gelled at body temperature can also be utilized. A variety of thermoreversible polymers are known, including natural gel-forming materials such as agarose, agar, furcellaran, beta-carrageenan, beta-1,3-glucans such as curdlan, gelatin, or polyoxyalkylene containing compounds, as described above. Specific examples include thermosetting biodegradable polymers for in vivo use described in U.S. Pat. No. 4,938,763, the contents of which are incorporated herein by reference.

Polymer Blends with Monomers and/or Oligomers

Polymers with enhanced bioadhesive properties are provided by incorporating anhydride monomers or oligomers into one of the polymers listed above by dissolving, dispersing, or blending, as taught by U.S. Pat. Nos. 5,955,096 and 6,156,348, the contents of which are incorporated herein by reference. The polymers may be used to form drug delivery systems which have improved ability to adhere to tissue surfaces, such as mucosal membranes. The anhydride oligomers are formed from organic diacid monomers, preferably the diacids normally found in the Krebs glycolysis cycle. Anhydride oligomers which enhance the bioadhesive properties of a polymer have a molecular weight of about 5000 or less, typically between about 100 and 5000 daltons, or include 20 or fewer diacid units linked by anhydride linkages and terminating in an anhydride linkage with a carboxylic acid monomer.

The oligomer excipients can be blended or incorporated into a wide range of hydrophilic and hydrophobic polymers including proteins, polysaccharides and synthetic biocompatible polymers, including those described above. In one embodiment, anhydride oligomers may be combined with metal oxide particles, such as those described above, to improve bioadhesion even more than with the organic additives alone. Organic dyes, because of their electronic charge and hydrophobicity or hydrophilicity, can either increase or decrease the bioadhesive properties of polymers when incorporated into the polymers.

As used herein, the term “anhydride oligomer” refers to a diacid or polydiacid linked by anhydride bonds, and having carboxy end groups linked to a monoacid such as acetic acid by anhydride bonds. The anhydride oligomers have a molecular weight less than about 5000, typically between about 100 and 5000 daltons, or are defined as including between one to about 20 diacid units linked by anhydride bonds. In one embodiment, the diacids are those normally found in the Krebs glycolysis cycle. The anhydride oligomer compounds have high chemical reactivity.

The oligomers can be formed in a reflux reaction of the diacid with excess acetic anhydride. The excess acetic anhydride is evaporated under vacuum, and the resulting oligomer, which is a mixture of species which include between about one to twenty diacid units linked by anhydride bonds, is purified by recrystallizing, for example, from toluene or other organic solvents. The oligomer is collected by filtration, and washed, for example, in ethers. The reaction produces anhydride oligomers of mono and poly acids with terminal carboxylic acid groups linked to each other by anhydride linkages.

The anhydride oligomer is hydrolytically labile. As analyzed by gel permeation chromatography, the molecular weight may be, for example, on the order of 200-400 for fumaric acid oligomer (FAPP) and 2000-4000 for sebacic acid oligomer (SAPP). The anhydride bonds can be detected by Fourier transform infrared spectroscopy by the characteristic double peak at 1750 cm⁻¹ and 1820 cm⁻¹, with a corresponding disappearance of the carboxylic acid peak normally at 1700 cm⁻¹.

In one embodiment, the oligomers may be made from diacids described for example in U.S. Pat. Nos. 4,757,128, 4,997,904 and 5,175,235, the disclosures of which are incorporated herein by reference. For example, monomers such as sebacic acid, bis(p-carboxy-phenoxy)propane, isophthalic acid, fumaric acid, maleic acid, adipic acid or dodecanedioic acid may be used.

Organic dyes, because of their electronic charge and hydrophilicity or hydrophobicity, may alter the bioadhesive properties of a variety of polymers when incorporated into the polymer matrix or bound to the surface of the polymer. A partial listing of dyes that affect bioadhesive properties include, but are not limited to: acid fuchsin, alcian blue, alizarin red s, auramine o, azure a and b, Bismarck brown y, brilliant cresyl blue ald, brilliant green, carmine, cibacron blue 3GA, congo red, cresyl violet acetate, crystal violet, eosin b, eosin y, erythro sin b, fast green fcf, giemsa, hematoylin, indigo carmine, Janus green b, Jenner's stain, malachite green oxalate, methyl blue, methylene blue, methyl green, methyl violet 2b, neutral red, Nile blue a, orange II, orange G, orcein, paraosaniline chloride, phloxine b, pyronin b and y, reactive blue 4 and 72, reactive brown 10, reactive green 5 and 19, reactive red 120, reactive yellow 2,3, 13 and 86, rose bengal, safranin o, Sudan III and IV, Sudan black B and toluidine blue.

Polymers Functionalized with Hydroxy-Substituted Aromatic Groups

Polymers having an aromatic group which contains one or more hydroxyl groups grafted onto them or coupled to individual monomers are also suitable for use in the bioadhesive coatings of the invention, as described in U.S. Provisional Application No. 60/528,042, filed Dec. 9, 2003, U.S. application Ser. No. 11/009,327, filed Dec. 9, 2004, and WO 2005/056708, the contents of which are incorporated herein by reference. Such polymers can be biodegradable or non-biodegradable polymers. The polymer can be hydrophobic. Preferably, the aromatic group is catechol or a derivative thereof and the polymer contains reactive functional groups, so that a hydroxyl-substituted aromatic group can be readily attached. Typically, the polymer is a polyanhydride and the aromatic compound is the catechol derivative DOPA. These materials display bioadhesive properties superior to conventional bioadhesives used in therapeutic and diagnostic applications.

As used herein “catechol moiety” refers to a moiety with the following generic structure:

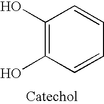

The molecular weight of the suitable polymers and percent substitution of the polymer with the aromatic group may vary greatly. The degree of substitution varies based on the desired adhesive strength, it may be as low as 10%, 25%, 30%, 40%, or 50%, or up to 100% substitution. Generally, about 10% to about 40%, such as about 20% to about 30% of the monomers in the polymeric backbone are substituted with at least one aromatic group. Preferably, about 100% of the monomers in the polymeric backbone are substituted with at least one aromatic group. The resulting polymer typically has a molecular weight ranging from about 1 to 2,000 kDa.

The polymer that forms that backbone of the bioadhesive material can be a biodegradable polymer. Examples of preferred biodegradable polymers include synthetic polymers such as poly hydroxy acids, such as polymers of lactic acid and glycolic acid, polyanhydrides, poly(ortho)esters, polyesters, polymethanes, poly(butyric acid), poly(valeric acid), poly(caprolactone), poly(hydroxybutyrate), poly(lactide-co-glycolide) and poly(lactide-cocaprolactone), and natural polymers such as alginate and other polysaccharides, collagen and chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, chitin, chitosan, pectin, amylopectin, copolymers and mixtures thereof. In general, these materials degrade either by enzymatic hydrolysis or exposure to water in vivo and by surface or bulk erosion. The foregoing materials may be used alone, as physical mixtures (blends), or as co-polymers.

Suitable polymers can formed by first coupling the aromatic compound to the monomer and then polymerizing. In this example, the monomers may be polymerized to form a polymer backbone, including biodegradable and non-biodegradable polymers. Suitable polymer backbones include, but are not limited to, polyanhydrides, polyamides, polycarbonates, polyalkylenes, polyalkylene oxides such as polyethylene glycol, polyalkylene terephthalates such as poly(ethylene terephthalate), polyvinyl alcohols, polyvinyl ethers, polyvinyl esters, polyethylene, polypropylene, poly(vinyl acetate), poly(vinyl chloride), polystyrene, polyvinyl halides, polyvinylpyrrolidone, polyhydroxy acids, polysiloxanes, polyurethanes and copolymers thereof, alkyl cellulose, hydroxyalkyl celluloses, cellulose ethers, cellulose esters, nitrocellulloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, and polyacrylates such as poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadccyl acrylate).

A suitable polymer backbone can be a known bioadhesive polymer that is hydrophilic or hydrophobic. Hydrophilic polymers include CARBOPOL™, polycarbophil, cellulose esters, and dextran.

Non-biodegradable polymers, especially hydrophobic polymers are also suitable as polymer backbones. Examples of preferred non-biodegradable polymers include ethylene vinyl acetate, poly(methacrylic acid), copolymers of maleic anhydride with other unsaturated polymerizable monomers, poly(butadiene maleic anhydride), polyamides, copolymers and mixtures thereof and dextran, cellulose and derivatives thereof.

Hydrophobic polymer backbones include polyanhydrides, poly(ortho)esters, and polyesters such as polycaprolactone. Preferably, the polymer is sufficiently hydrophobic that it is not readily water soluble, for example the polymer should be soluble up to less than about 1% w/w in water, preferably about 0.1% w/w in water at room temperature or body temperature. In the most preferred embodiment, the polymer is a polyanhydride, such as a poly(butadiene maleic anhydride) or another copolymer of maleic anhydride. Polyanhydrides may be formed from dicarboxylic acids as described in U.S. Pat. No. 4,757,128 to Domb et al., incorporated herein by reference. Suitable diacids include aliphatic dicarboxylic acids, aromatic dicarboxylic acids, aromatic-aliphatic dicarboxylic acid, combinations of aromatic, aliphatic and aromatic-aliphatic dicarboxylic acids, aromatic and aliphatic heterocyclic dicarboxylic acids, and aromatic and aliphatic heterocyclic dicarboxylic acids in combination with aliphatic dicarboxylic acids, aromatic-aliphatic dicarboxylic acids, and aromatic dicarboxylic acids of more than one phenyl group. Suitable monomers include sebacic acid (SA), fumaric acid (FA), bis(p-carboxyphenoxy)propane (UP), isophthalic acid (IPh), and dodecanedioic acid (DD).

A wide range of molecular weights are suitable for the polymer that forms the backbone of the bioadhesive material. The molecular weight may be as low as about 200 Da (for oligomers) up to about 2,000 kDa. Preferably the polymer has a molecular weight of at least 1,000 Da, more preferably at least 2,000 Da, most preferably the polymer has a molecular weight of up to 20 kDa or up to 200 kDa. The molecular weight of the polymer may be up to 2,000 kDa (e.g., 20 kDa to 1,000 kDa or 2,000 kDa).

The range of substitution on the polymer can vary greatly and depends on the polymer used and the desired bioadhesive strength. For example, a butadiene maleic anhydride copolymer that is 100% substituted with DOPA will have the same number of DOPA molecules per chain length as a 67% substituted ethylene maleic anhydride copolymer. Typically, the polymer has a percentage substitution ranging from 10% to 100%, preferably ranging from 50% to 100%.

The polymers and copolymers that form the backbone of the bioadhesive material include reactive functional groups that interact with the functional groups on the aromatic compound.

It is important that the polymer or monomer that forms the polymeric backbone contains accessible functional groups that easily react or interact with molecules contained in the aromatic compounds, such as amines and thiols. In a preferred embodiment, the polymer contains amino reactive moieties, such as aldehydes, ketones, carboxylic acid derivatives, cyclic anhydrides, alkyl halides, aryl asides, isocyanates, isothiocyanates, succinimidyl esters or a combination thereof.

Preferably, the aromatic compound containing one or more hydroxyl groups is catechol or a derivative thereof. Optionally the aromatic compound is a polyhydroxy aromatic compound, such as a trihydroxy aromatic compound (e.g., phloroglucinol) or a multihydroxy aromatic compound (e.g., tannin). The catechol derivative may contain a reactive group, such as an amino, thiol, or halide group. The preferred catechol derivative is 3,4-dihydroxyphenylalanine (DOPA), which contains a primary amine. Tyrosine, the immediate precursor of DOPA, which differs only by the absence of one hydroxyl group in the aromatic ring, can also be used. Tyrosine is capable of conversion (e.g., by hydroxylation) to the DOPA form. A particularly preferred aromatic compound is an amine-containing aromatic compound, such as an amine-containing catechol derivative (e.g., dopamine).

Two general methods are used to form the polymer product. In one example, a compound containing an aromatic group which contains one or more hydroxyl groups is grafted onto a polymer. In this example, the polymeric backbone is a biodegradable polymer. In a second example, the aromatic compound is coupled to individual monomers and then polymerized.

Any chemistry which allows for the conjugation of a polymer or monomer to an aromatic compound containing one or more hydroxyl groups can be used, for example, if the aromatic compound contains an amino group and the monomer or polymer contains an amino reactive group, this modification to the polymer or monomer is performed through a nucleophilic addition or a nucleophilic substitution reaction, such as a Michael-type addition reaction, between the amino group in the aromatic compound and the polymer or monomer. Additionally, other procedures can be used in the coupling reaction. For example, carbodiimide and mixed anhydride based procedures form stable amide bonds between carboxylic acids or phosphates and amino groups, bifunctional aldehydes react with primary amino groups, bifunctional active esters react with primary amino groups, and divinylsulfone facilitates reactions with amino, thiol, or hydroxy groups.

The aromatic compounds are grafted onto the polymer using standard techniques to form the bioadhesive material. In one example, L-DOPA is grafted to maleic anhydride copolymers by reacting the free amine in L-DOPA with the maleic anhydride bond in the copolymer.

A variety of different polymers can be used as the backbone of the bioadhesive material, as described above. Additional representative polymers include 1:1 random copolymers of maleic anhydride with ethylene, vinyl acetate, styrene, or butadiene. In addition, a number of other compounds containing aromatic rings with hydroxy substituents, such as tyrosine or derivatives of catechol, can be used in this reaction.

In another embodiment, the polymers are prepared by conjugate addition of a compound containing an aromatic group that is attached to an amine to one or more monomers containing an amino reactive group. In a preferred method, the monomer is an acrylate or the polymer is acrylate. For example, the monomer can be a diacrylate such as 1,4-butanediol diacrylate, 1,3-propanediol diacrylate, 1,2-ethanediol diacrylate, 1,6-hexanediol diacrylate, 2,5-hexanediol diacrylate or 1,3-propanediol diacrylate. In an example of the coupling reaction, the monomer and the compound containing an aromatic group are each dissolved in an organic solvent (e.g., THF, CH₂Cl₂, methanol, ethanol, CHCl₃, hexanes, toluene, benzene, CCl₄, glyme, diethyl ether, etc.) to form two solutions. The resulting solutions are combined, and the reaction mixture is heated to yield the desired polymer. The molecular weight of the synthesized polymer can be controlled by the reaction conditions (e.g., temperature, starting materials, concentration, solvent, etc) used in the synthesis.

For example, a monomer, such as 1,4-phenylene diacrylate or 1,4-butanediol diacrylate having a concentration of 1.6 M, and DOPA or another primary amine containing aromatic molecule are each dissolved in an aprotic solvent such as DMF or DMSO to form two solutions. The solutions are mixed to obtain a 1:1 molar ratio between the diacrylate and the amine group and heated to 56° C. to form a bioadhesive material.

### Coatings

Preferred bioadhesive coatings do not appreciably swell upon hydration, such that they do not substantially inhibit or block movement (e.g., of ingested food) through the gastrointestinal tract, as compared to the polymers disclosed by Duchene et al. Generally, polymers that do not appreciably swell upon hydration include one or more hydrophobic regions, such as a polymethylene region (e.g., (CH₂)ₙ, where n is 4 or greater). The swelling of a polymer can be assessed by measuring the change in volume when the polymer is exposed to an aqueous solution. Polymers that do not appreciably swell upon hydration expand in volume by 50% or less when fully hydrated. Preferably, such polymers expand in volume by less than 25%, less than 20%, less than 15%, less than 10% or less than 5%. Even more preferably, the bioadhesive coatings are mucophilic.

In one embodiment, a polymer that does not appreciably swell upon hydration (e.g., a hydrophobic polymer) is mixed or blended with a polymer that does swell or a hydrophilic substance (e.g., Carbopol™, poly(acrylic acid), small organic acids such as citric acid, maleic acid, fumaric acid, hydrophilic drugs, ionic and non-ionic detergents, sugars, salts such as NaCl, disintegrants), provided that the amount of swelling or hydration in the polymer does not substantially interfere with bioadhesiveness. Generally, the amount of swellable polymer or hydrophilic substance is selected to sufficiently hydrate the non-swellable polymer to enhance its bioadhesiveness. The weight ratio of swellable to non-swellable polymer or hydrophilic substance to non-swellable polymer can be varied in order to obtain a coating that combines a desired amount of swelling (e.g., for faster adhesion) with longer-lasting adhesion, such as from 5:1 to 1:5 or 2:1 to 1:2. For example, the swellable polymer and/or hydrophilic substance can comprise about 1% to about 30% by weight of a bioadhesive coating.

In one embodiment, the bioadhesive polymeric coating consists of two layers, an inner bioadhesive layer that does not substantially swell upon hydration and an outer bioadhesive layer that is readily hydratable and optionally bioerodable, such as one comprised of Carbopol™.

The bioadhesive polymers discussed above can be mixed with one or more plasticizers or thermoplastic polymers. Such agents typically increase the strength and/or reduce the brittleness of polymeric coatings. Examples of plasticizers include dibutyl sebacate, polyethylene glycol, triethyl citrate, dibutyl adipate, dibutyl fumarate, diethyl phthalate, ethylene oxide-propylene oxide block copolymers such as Pluronic™ F68 and di(sec-butyl)fumarate. Examples of thermoplastic polymers include polyesters, poly(caprolactone), polylactide, poly(lactide-co-glycolide), methyl methacrylate (e.g., EUDRAGIT™), cellulose and derivatives thereof such as ethyl cellulose, cellulose acetate and hydroxypropyl methyl cellulose (HPMC) and large molecular weight polyanhydrides. The plasticizers and/or thermoplastic polymers are mixed with a bioadhesive polymer to achieve the desired properties. Typically, the proportion of plasticizers and thermoplastic polymers, when present, is from 0.5% to 40% by weight.

In one embodiment, the bioadhesive polymer coating, in a dry packaged form of a tablet, is a hardened shell.

A tablet or a drug eluting device can have one or more coatings in addition to the bioadhesive polymeric coating, e.g., covering the surface of the bioadhesive coating. These coatings and their thickness can, for example, be used to control where in the gastrointestinal tract the bioadhesive coating becomes exposed. In one example, the additional coating prevents the bioadhesive coating from contacting the mouth or esophagus. In another example, the additional coating remains intact until reaching the small intestine (e.g., an enteric coating).

Examples of coatings include methylmethacrylates, zein, cellulose acetate, cellulose phthalate, HMPC, sugars, enteric polymers, gelatin and shellac. Premature dissolution of a tablet in the mouth can be prevented with hydrophilic polymers such as HPMC or gelatin.

Coatings used in tablets of the invention typically include a pore former such that the coating is permeable to the drug.

### Excipients

The cores of tablets and drug eluting devices of the invention may contain one or more excipients, carriers or diluents. These excipients, carriers or diluents can be selected, for example, to control the disintegration rate of a tablet or drug eluting device. In particular, it is advantageous for the disintegration time to be less than the gastric (or small/large intestinal) retention time. In one embodiment, the disintegration time of a tablet is at least 25% of the gastric retention time, at least 50% of the gastric retention time or at least 75% of the gastric retention time.

It will be understood by those skilled in the art that any vehicle or carrier conventionally employed and which is inert with respect to the active agent, and preferably does not interfere with bioadhesiveness, may be utilized for preparing and administering the pharmaceutical compositions of the present invention. Illustrative of such vehicles and carriers are those described, for example, in Remington's Pharmaceutical Sciences, 18th ed. (1990), the disclosure of which is incorporated herein by reference.

The formulations of the present invention for use in a subject comprise one or more drugs, optionally together with one or more acceptable carriers or diluents therefor and optionally other therapeutic ingredients. The carriers or diluents must be “acceptable” in the sense of being compatible with the other ingredients of the formulation and not deleterious to the recipient thereof. The formulations can conveniently be presented in unit dosage form and can be prepared by any of the methods well known in the art of pharmacy. All methods include the step of bringing into association the drug with the carrier or diluent which constitutes one or more accessory ingredients. In general, the formulations are prepared by uniformly and intimately bringing into association the agent with the carriers and then, if necessary, dividing the product into unit dosages thereof.

Examples of carriers and diluents include pharmaceutically accepted hydrogels such as alginate, chitosan, methylmethacrylates, cellulose and derivatives thereof (microcrystalline cellulose, hydroxypropyl cellulose, hydroxypropyl methyl cellulose, carboxymethylcellulose, ethylcellulose), agarose and Povidone™, kaolin, magnesium stearate, starch, lactose, sucrose, density-controlling agents such as barium sulfate and oils, dissolution enhancers such as aspartic acid, citric acid, glutamic acid, tartartic acid, sodium bicarbonate, sodium carbonate, sodium phosphate, glycine, tricine and TRIS.

For multi-layer tablets in particular, the tablet typically includes at least one polymer or excipient. The polymer may be degradable or non-degradable. Suitable degradable polymers include polyesters, such as poly(lactic acid) (p[LA]), poly(lactide-co-glycolide) (p[LGA]), poly(caprolactone) (p[CL]); polyanhydrides such as poly(fumaric-co-sebacic anhydride) (p[FASA]) in molar ratios of 20:80 to 90:10, poly(carboxyphenoxypropane-co-sebacic anhydride) (p[CPPSA]), poly(adipic anhydride) (p[AA]); polyorthoesters; polyamides; and polyimides. Other suitable polymers include hydrogel-based polymers such as agarose, alginate, and chitosan. Suitable non-degradable polymers include polystyrene, polyvinylphenol, and polymethylmethacrylates (Eudragits™).

The excipients, carriers or diluents can also be selected to control the time until a tablet or drug eluting device detaches from a mucosal membrane. In particular, the addition of one or more disintegrating agents will reduce the time until a tablet or drug eluting device detaches. Alternatively or in combination with the disintegrating agents, an agent that interferes with the mucosa-tablet/device adhesion can be used to control the time until detachment occurs.

Suitable excipients include stabilizers, plasticizers, wetting agents, antitack agents, tack agents, foam agents, antifoam agents, binders, fillers, extenders, flavorants, dispersants, surfactants, solubilizers, solubilization inhibitors, glidants, lubricants, antiadherents, adherents, coatings, protective agents, sorbents, suspending agents, crystallization inhibitors, recrystallization inhibitors, disintegrants, acidulants, diluents, alkalizing agents, antioxidants, preservatives, colorants, electrolytes, solvents, antisolvents, accelerating agents, and/or retarding agents. Examples include alginate, chitosan, methylmethacrylates (Eudragits™), celluloses (especially microcrystalline cellulose, hydroxypropylmethylcellulose, ethylcellulose etc), agarose, Povidone™, lactose, microcrystalline cellulose, kaolin starch, magnesium stearate, stearic acid, glycerol monostearate, sucrose, compressible sugar, lactose and barium sulfate.

The present invention can be further understood by reference to the following non-limiting examples.

### EXEMPLIFICATION

### Example 1

### Bioadhesion Assay

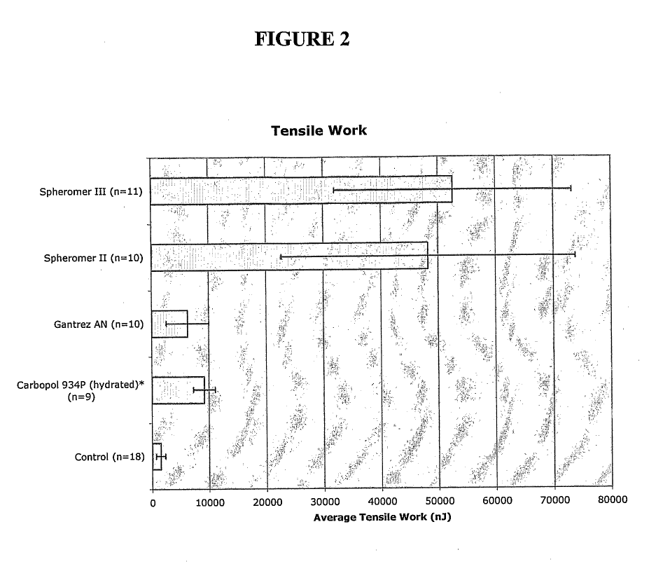

The bioadhesion of Spheromer™ II, Spheromer™ III, Gantrez® AN-119 BF (a copolymer of 2,5-Furandione and methoxyethene) and hydrated Carbopol® 934P NF (a cross-linked polyacrylic acid homopolymer) films, prepared by dip-coating on nylon supports, was tested using a Texture Analyzer TA XT II tensile tester, with pig intestine as the biological substrate. The parameters measured were fracture strength (peak force of detachment normalized for cross-sectional surface area) and tensile work (area under the deformation vs. load curve). Results are depicted in FIGS. 1 and 2.

Polymer films on supports were prepared by dip-coating in concentrated polymers solution and drying. Twenty percent (w/v) solutions were made for all test materials except for Carbopol® 934P, which was a 2% (w/v) solution in water. Spheromer™ II was dissolved with an equal amount of Eudragit® RL 100 in dichloromethane. The films on supports were air-dried for 24 hrs after dipping and lyophilized overnight to remove residual solvents.

Pig intestine was cut into at least 1 in 2 sections, mounted into a perforated, plastic holder with the mucus side up and submerged in phosphate buffered saline (PBS, pH 6.8). A fresh piece of tissue was used for each test. A polymer-coated support was mounted on the Texture Analyzer and brought into contact with the pig intestine sample. An uncoated support was used as the control. After 7 minutes, the support was lifted away from the sample tissue and the load vs. deformation curve was plotted. Instrumental settings are listed in the table below:

Texture Analyzer Settings

| CAPTION | VALUE | UNITS |
|---|---|---|
| Pre-Test Speed | 0.50 | mm/sec |
| Test Speed | 0.50 | mm/sec |
| Post-Test Speed | 0.50 | mm/sec |
| Force | 5.0 | g |
| Time | 420.00 | sec |
| Trigger Type | Button | — |
| Trigger Force | 5.0 | g |
| Trigger Distance | 0.000 | mm |

### Example 2

### Longitudinally Compressed Tablets Containing 250 mg Valacyclovir HCl (Lot #502-094)

Longitudinally compressed core tablets (LCT) were prepared by using a pair of 0.2618″ dies (Natoli Engineering). The compound die was filled with 250 mg of a Valacyclovir immediate release (IR) dry blend. The tablets were prepared by direct compaction at 500 psi for 1 second using standard 0.2618″ upper/lower punches and the GlobePharma Manual Tablet Compaction Machine (MTCM-1). Each tablet contained 250 mg Valacyclovir. The composition of core tablets is provided in Table 2.

The core tablets were first coated peripherally with an impermeable, solvent-cast film of polycaprolactone (PCL, MW 200 kDA) that was attached to the tablet by heat sealing at 40-60° C. Then, a second film comprising bioadhesive Spheromer II™ (Fumaric Anhydride Oligomer) blended in polycaprolactone was applied over the first film. Fumaric anhydride oligomer, polycaprolactone (MW 200 kDa) and dibutyl sebacate, in the 40%:50%:10% w/w ratio, were co-dissolved in dichloromethane and a film was prepared by solvent-casting on a tray. After 24 hrs of drying, the dry, cut film was applied over the existing, impermeable, PCL coating and the edges of the film were joined by heating. The longitudinal-section of these dosage forms is illustrated in FIG. 3.

TABLE 2
Composition of Valacyclovir HCI 250 mg Core Tablet Formulation

| Ingredients | IR Layer |
|---|---|
| Valacyclovir* | 277.8 |
| Hydroxypropyl cellulose | 75.0 |
| Compressible Sugar | 132.7 |
| Crospovidone, NF | 12.5 |
| Magnesium Stearate | 2.0 |
| Total | 500.0 |

*277.8 mg Valacyclovir HCl is equivalent to 250 mg Valacyclovir

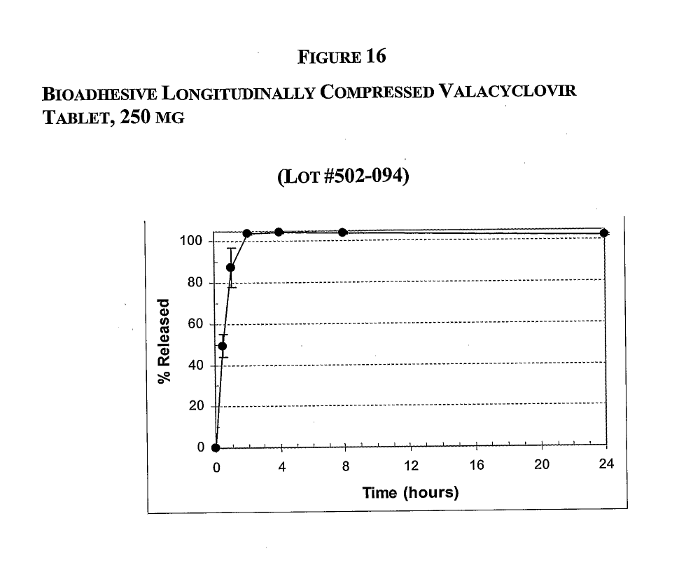

The bioadhesive-coated tablets were tested for release profile in 0.1 N HCl at 37±0.5° C., in the USP II dissolution apparatus at 100 rpm. The in vitro dissolution profile of tablets is shown in FIG. 16. As shown in FIG. 16, 50% of the valacyclovir is released in 30 minutes, and all of it is released by 2 hours.

It should be evident to those skilled in the art that drug release from LCT's covered with an impermeable first coat of PCL (or other impermeable polymer such as ethyl cellulose, cellulose acetate, zein etc) and a second, overlying, bioadhesive coat of PCL and Spheromer™ II should be identical to that of LCT's coated only with a single layer of impermeable polymer. The bioadhesive coating facilitates intimate contact of the dosage form with GI mucosa, enabling prolonged retention in GIT and shortened diffusion distances for drug absorption.

### Example 3

### Longitudinally Compressed Tablets Containing 400 mg Gabapentin (Lot # 411-104, 412-047 and 412-006)

Longitudinally compressed core tablets (LCTs) were prepared by using a pair of 0.2900″ dies (Natoli Engineering). The dies were carefully aligned and firmly joined together to form a compound die. The compound die was filled with 800 mg of a dry blend of drug and excipients. The tablets were prepared by direct compaction at 4000 psi for 1 second using standard 0.2900″ upper/lower punches and a GlobePharma Manual Tablet Compaction Machine (MTCM-1). Each tablet contained 400 mg Gabapentin. The composition of core tablets is provided in Table 3.

The LCTs were coated peripherally first with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of these dosage forms is illustrated in FIG. 3.

TABLE 3
Composition of Gabapentin 400 mg LCT Core Tablet Formulations

| | Weight (mg) | | |
|---|---|---|---|
| Ingredients | 411-104 | 412-047 | 412-006 |
| Gabapentin | 400.0 | 400.0 | 400.0 |
| Hydroxypropyl cellulose | 200.0 | 200.0 | 200.0 |
| Polyethylene oxide | 120.0 | 80.0 | 80.0 |
| Hydroxypropylmethyl | 0 | 40.0 | 60.0 |
| cellulose | | | |
| Lactose | 72.8 | 72.8 | 52.8 |
| Polyvinylpyrrolidone | 4.0 | 4.0 | 4.0 |
| Magnesium Stearate | 3.2 | 3.2 | 3.2 |
| Total | 800.0 | 800.0 | 800.0 |

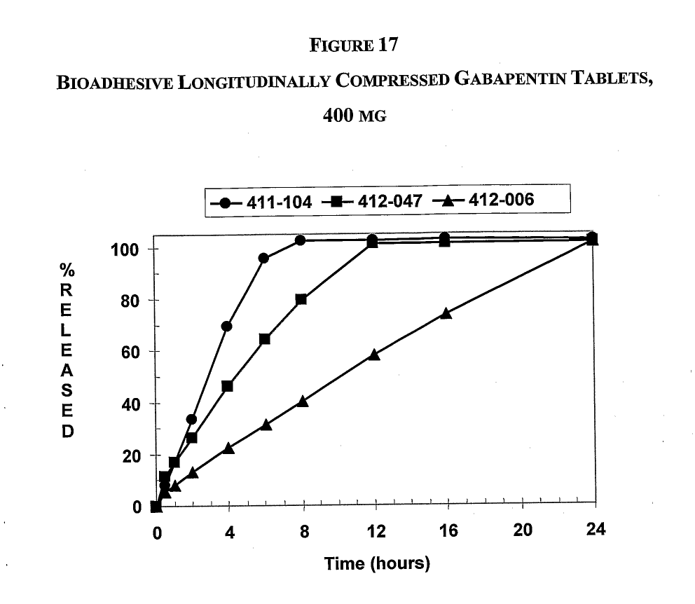

Coated Gabapentin tablets were tested for release profile in 0.1HCl at 37±0.5° C., in the USP II dissolution apparatus at 100 rpm. The in vitro release profile of tablets is shown in FIG. 17. As shown in FIG. 17, about 35% of the gabapentin in the Lot # 411-104 formulation is released in 2 hours, 70% by 4 hours, about 95% by 6 hours, and all of it is released by 8 hours. The release profile of the Lot # 412-047 gabapentin formulation is about 25% of the drug by 2 hours, almost 50% by 4 hours, 80% by 8 hours, and all of it by 12 hours. Gabapentin is released from the third formulation (Lot #412-006) in the following manner: 20% by 4 hours, about 40% by 8 hours, about 70% by 16 hours, and 100% by 24 hours.

### Example 4

### Longitudinally Compressed Tablets Containing 500 mg Gabapentin (Lot #411-029, 411-106 and 411-108)

Longitudinally compressed core tablets were prepared by using a pair of 0.2900″ dies (Natoli Engineering). The compound die was filled with 800 mg of a dry blend of drug and excipients. The tablets were prepared by direct compaction at 4000 psi for 1 second using standard 0.2900″ upper/lower punches and the GlobePharma Manual Tablet Compaction Machine (MTCM-1). Each tablet contained 500 mg Gabapentin. The compositions of core tablets are provided in Table 4.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers) or combinations of these polymers can also be applied over the impermeable coating without affecting drug release. The longitudinal-section of these dosage forms is illustrated in FIG. 3.

TABLE 4
Composition of Gabapentin 500 mg Core Tablet Formulations

| | Weight (mg) | | |
|---|---|---|---|
| Ingredients | 411-029 | 411-106 | 411-108 |
| Gabapentin | 500.0 | 500.0 | 500.0 |
| Hydroxypropyl cellulose | 200.0 | 200.0 | 200.0 |
| Hydroxypropylmethyl | 60.0 | 40.0 | 20.0 |
| cellulose | | | |
| Polyethylene oxide | 36.8 | 56.8 | 76.8 |
| Magnesium Stearate | 3.2 | 3.2 | 3.2 |
| Total | 800.0 | 800.0 | 800.0 |

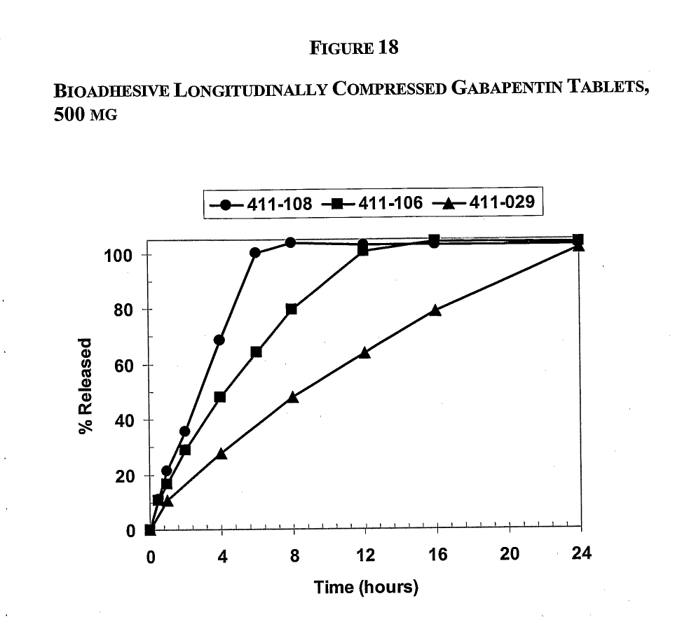

Coated Gabapentin tablets were tested for release profile in 0.1 N HCl at 37±0.5° C., in the USP II dissolution apparatus at 100 rpm. The in vitro release profiles of tablets are shown in FIG. 18. As shown in FIG. 18, a little more than 20% of the gabapentin in the Lot # 411-108 formulation is released in 1 hour, 35% by 2 hours, about 70% by 4 hours, and all of it is released by 6 hours. The release profile of the Lot # 411-106 gabapentin formulation is about almost 30% of the drug by 2 hours, almost 50% by 4 hours, almost 80% by 8 hours, and all of it by 12 hours. Gabapentin is released from the third formulation (Lot # 411-029) in the following manner: 10% by 1 hour, a little more than 25% by 4 hours, almost 50% by 8 hours, a little more than 60% by 12 hours, about 75% by 16 hours, and 100% by 24 hours.

### Example 5

### Longitudinally Compressed Tablets Containing 225 mg Valacyclovir (Lot #502-063, 502-065 and 502-067)

Longitudinally compressed core tablets were prepared by using a pair of 0.2618″ dies (Natoli Engineering). The compound die was filled with 500 mg of a dry blend of drug and excipients. The tablets were prepared by direct compaction at 4000 psi for 1 second using standard 0.2618″ upper/lower punches and the GlobePharma Manual Tablet Compaction Machine (MTCM-1). Each tablet contained 225 mg Valacyclovir. The compositions of core tablets are provided in Table 5.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of these dosage forms is illustrated in FIG. 3.

TABLE 5
Composition of Valacyclovir 225 mg LCT Core Tablet
Formulations

| | Weight (mg) | | |
|---|---|---|---|
| Ingredients | 502-063 | 502-065 | 502-067 |
| Valacyclovir HCl* | 250.0 | 250.0 | 250.0 |
| Hydroxypropyl cellulose | 125.0 | 125.0 | 125.0 |
| Lactose | 58.0 | 45.5 | 33.0 |
| Polyethylene oxide | 50.0 | 50.0 | 50.0 |
| Hydroxypropylmethyl | 12.5 | 25.0 | 37.5 |
| cellulose | | | |
| Polyvinylpyrolidone | 2.5 | 2.5 | 2.5 |
| Magnesium Stearate | 2.0 | 2.0 | 2.0 |
| Total | 500.0 | 500.0 | 500.0 |

*Equivalent to 225 mg Valacyclovir

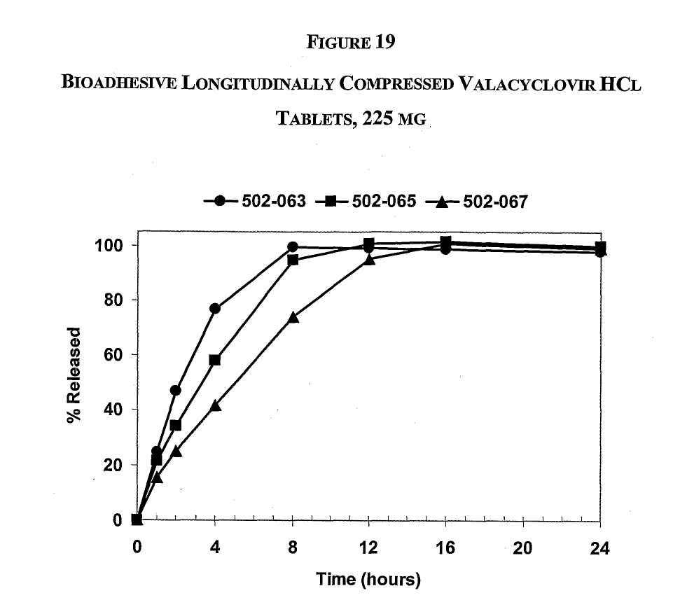

Coated Valacyclovir HCl tablets were tested for release profiles in 0.1 N HCl at 37±0.5° C., in the USP II dissolution apparatus at 100 rpm. The in vitro release profiles of tablets are shown in FIG. 19. As shown in FIG. 19, about 25% of the valacyclovir in the Lot # 502-063 formulation is released in 1 hour, almost 50% by 2 hours, about 75% by 4 hours, and all of it is released by 8 hours. The release profile of the Lot # 502-065 valacyclovir formulation is a little more than 20% of the drug by 1 hour, a little more than 30% by 2 hours, almost 60% by 4 hours, about 95% by 8 hours, and all of it by 12 hours. Valacyclovir is released from the third formulation (Lot # 502-067) in the following manner: about 15% by 1 hour, almost 25% by 2 hours, a little more than 40% by 4 hours, about 75% by 8 hours, and 100% by 16 hours.

### Example 6

### Longitudinally Compressed Tablets Containing 450 mg Valacyclovir (Lot #502-069, 502-071 and 502-073)

Longitudinally compressed core tablets were prepared by using a pair of 0.2900″ dies (Natoli Engineering). The compound die was filled with 800 mg of a dry blend of drug and excipients. The tablets were prepared by direct compaction at 4000 psi for 1 second using standard 0.2900″ upper/lower punches and the GlobePharma Manual Tablet Compaction Machine (MTCM-1). Each tablet contained 500 mg Valacyclovir HCl. The compositions of core tablets are provided in Table 6.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of these dosage forms is illustrated in FIG. 3.

TABLE 6
Composition of Valacyclovir 450 mg Core Tablet Formulations

| | Weight (mg) | | |
|---|---|---|---|
| Ingredients | 502-069 | 502-071 | 502-073 |
| Valacyclovir HCl* | 500.0 | 500.0 | 500.0 |
| Hydroxypropyl cellulose | 200.0 | 200.0 | 200.0 |
| Polyethylene oxide | 96.8 | 76.8 | 56.8 |
| Hydroxypropylmethyl | — | 20.0 | 40.0 |
| cellulose | | | |
| Magnesium Stearate | 3.2 | 3.2 | 3.2 |
| Total | 800.0 | 800.0 | 800.0 |

*Equivalent to 450 mg Valacyclovir

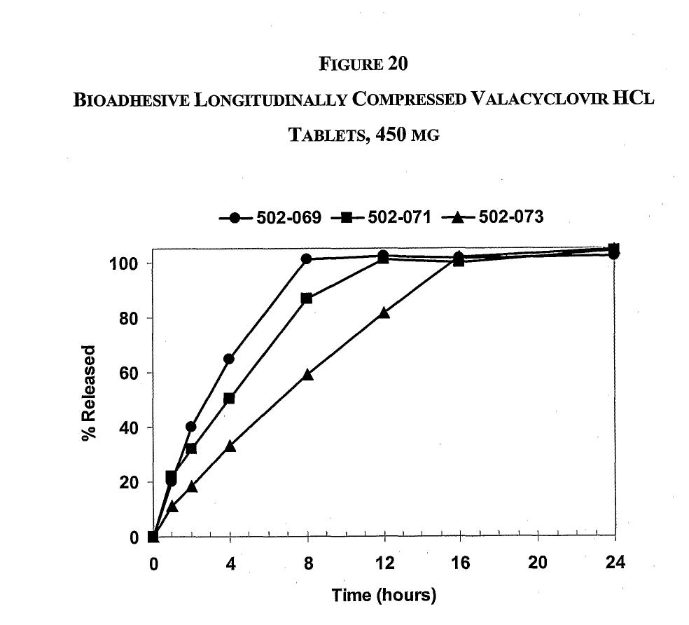

Coated Valacyclovir HCl tablets were tested for release profile in 0.1 N HCl at 37±0.5° C., in the USP II dissolution apparatus at 100 rpm. The in vitro release profiles of tablets are shown in FIG. 20. As shown in FIG. 20, about 20% of the valacyclovir in the Lot # 502-069 formulation is released in 1 hour, about 40% by 2 hours, about 65% by 4 hours, and all of it is released by 8 hours. The release profile of the Lot # 502-071 valacyclovir formulation is about 20% of the drug in 1 hour, about 50% by 4 hours, almost 90% by 8 hours, and all of it by 12 hours. Valacyclovir is released from the third formulation (Lot # 502-073) in the following manner: about 10% by 1 hour, a little more than 30% by 4 hours, 80% in 12 hours, and 100% by 16 hours.

### Example 7

### Longitudinally Compressed Tablets containing 500 mg Metformin HCl (Lot # 412-089, 412-091, 412-093 and 412-099)

Longitudinally compressed core tablets were prepared by using a pair of 0.2900″ dies (Natoli Engineering). The die was filled with 800 mg of a dry blend of drug and excipients. The tablets were prepared by direct compaction at 4000 psi for 1 second using standard 0.2900″ upper/lower punches and a GlobePharma Manual Tablet Compaction Machine (model MTCM-1). Each tablet contained 500 mg Metformin HCl. The core compositions of tablets are provided in Table 7.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers) or combinations of these polymers can also be applied over the impermeable coating. The cross-section of this dosage form is illustrated in FIG. 3.

TABLE 7
Composition of Metformin HCl 500 mg Core Tablet
Formulations

| | Weight (mg) | | | |
|---|---|---|---|---|
| Ingredients | 412-089 | 412-091 | 412-093 | 412-099 |
| Metformin HCl | 500.0 | 500.0 | 500.0 | 500.0 |
| Hydroxypropyl cellulose | 200.0 | 200.0 | 200.0 | 200.0 |
| Hydroxypropylmethyl cellulose | 76.8 | 56.8 | 60.0 | 60.0 |
| Polyethylene oxide | 20.0 | 40.0 | 36.8 | — |
| Polyvinylpyrrolidone (Povidone) | — | — | — | 36.8 |
| Magnesium Stearate | 3.2 | 3.2 | 3.2 | 3.2 |
| Total | 800.0 | 800.0 | 800.0 | 800.0 |

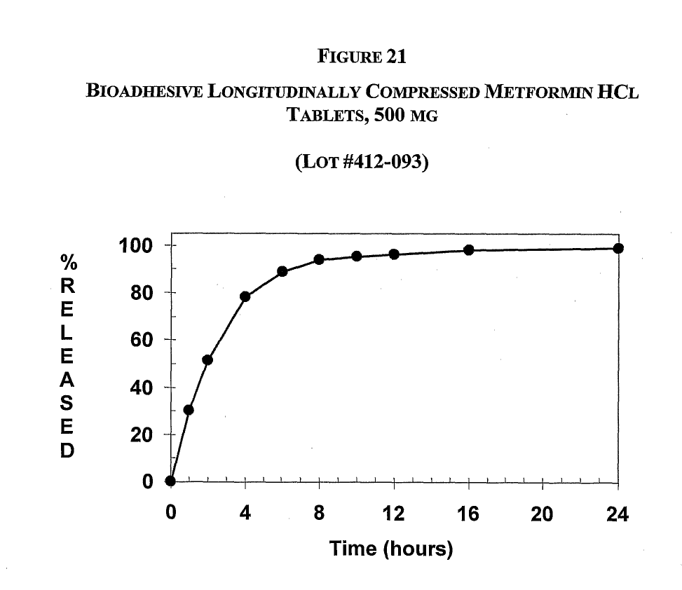

Coated metformin tablets were tested for release profiles in 0.1 N HCl at 37±0.5° C., in the USP II dissolution apparatus at 100 rpm. The in vitro dissolution profile of tablets from Lot #412-093 is shown in FIG. 21. As shown in FIG. 21, 30% of the metformin HCl in the Lot #412-093 formulation is released in 1 hour, a little more than 50% by 2 hours, almost 80% by 4 hours, almost 90% by 6 hours, and all of it is released by 24 hours.

### Example 8

### Longitudinally Compressed Tablets containing 30 mg Pioglitazone HCl (Lot #503-036)

Longitudinally compressed tablets were prepared by using a pair of 0.2618″ dies joined together in a similar fashion to the method described in Example 2. The compound die was filled with 500 mg of a dry blend of drug and excipients. The tablets were prepared by direct compaction at 500 psi for 1 second using a standard 0.2618″ lower punch, a special 0.2618″ upper punch (2″ tip length), and a manual tablet press in a similar fashion to the method described in Example 2. Each tablet contained 30 mg Pioglitazone HCl. The core composition of tablet is provided in Table 8.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release.

TABLE 8
Composition of Pioglitazone HCl 30 mg Core Tablet Formulation

| | Weight |
|---|---|
| Ingredients | (mg) |
| Pioglitazone HCl | 30.0 |
| Lactose | 325.0 |
| Hydroxypropyl cellulose | 125.0 |
| Hydroxypropylmethyl | 17.5 |
| cellulose | |
| Magnesium Stearate | 2.5 |
| Total | 500.0 |

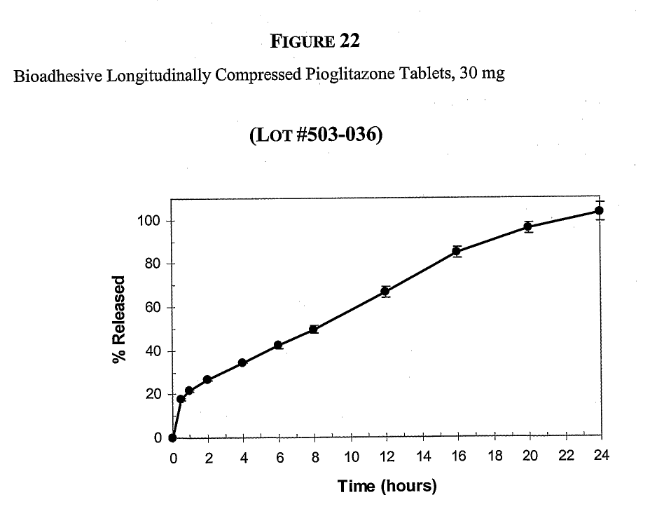

Coated pioglitazone tablets were tested for release profiles in 0.1 N HCl buffer containing 0.3 N KCl at 37±0.5° C., in the USP II dissolution apparatus at 100 rpm. The in vitro dissolution profiles of tablets containing 30 mg of Pioglitazone HCl are shown in FIG. 22. As shown in FIG. 22, almost 20% of the Pioglitazone in the Lot # 503-036 formulation is released in 30 minutes, a little more than 20% in 1 hour, 50% by 8 hours, 80% by 16 hours, and all of it is released by 24 hours.

### Example 9

### Longitudinally Compressed Tablet containing 30 mg Pioglitazone HCl (Lot #503-203)

Longitudinally compressed tablets were prepared by using a pair of 0.2618″ dies joined together in a similar fashion to the method described in Example 2. The compound die was filled with 300 mg of a dry blend of drug and excipients. The tablets were prepared by direct compaction at 500 psi for 1 second using a standard 0.2618″ lower punch, a special 0.2618″ upper punch (2″ tip length), and a manual tablet press described in Example 2. Each tablet contained 30 mg Pioglitazone HCl.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 3. The core composition of tablet is provided Table 9.

TABLE 9
Composition of Pioglitazone HCl 30 mg Core Tablet Formulation

| Ingredients | Weight (mg) |
|---|---|
| Pioglitazone HCl | 30.0 |
| Lactose | 261.0 |
| Croscarmellose Sodium | 7.5 |
| Magnesium Stearate | 1.5 |
| Total | 300.0 |

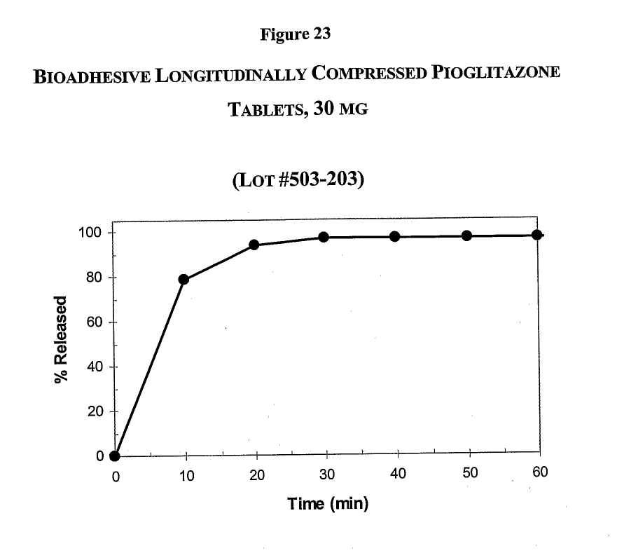

Coated pioglitazone tablets were tested for release profile in 0.1 N HCl buffer containing 0.3 N KCl at 37±0.5° C., in the USP II dissolution apparatus at 100 rpm. The in vitro dissolution profile of tablets containing 30 mg of Pioglitazone is shown in FIG. 23. As shown in FIG. 23, almost 80% of the Pioglitazone is released in 10 minutes, a little more than 90% in 20 minutes, and the release profile plateaus at 30 minutes.

### Example 10

### Longitudinally Compressed Tablets Containing 500 mg Valacyclovir HCl (Lot #501-213)

Longitudinally compressed bilayer core tablets were prepared by using a pair of 0.2900″ dies (Natoli Engineering). The compound die was filled first with 650 mg of a Valacyclovir controlled release (CR) dry blend, and then with 150 mg of a Valacyclovir immediate release (IR) dry blend. The tablets were prepared by direct compaction at 4000 psi for 1 second using standard 0.2900″ upper/lower punches and the GlobePharma Manual Tablet Compaction Machine (MTCM-1). Each tablet contained 450 mg Valacyclovir HCl.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of these dosage forms is illustrated in FIG. 4. The core composition of tablet is provided in Table 10.

TABLE 10
Composition of Valacyclovir HCl 500 mg Core Tablet Formulation

Weight (mg)

| Ingredients | CR Layer | IR Layer |
|---|---|---|
| Valacyclovir HCl* | 400.00 | 100.00 |
| Hydroxypropyl cellulose | 162.50 | — |
| Hydroxypropylmethyl cellulose | 48.75 | — |
| Polyethylene oxide | 36.14 | — |
| Compressible Sugar | — | 26.90 |
| Polyvinypyrrolidone | — | 22.50 |
| Magnesium Stearate | 2.60 | 0.60 |
| Total | 650.0 | 150.0 |

*Equivalent to 450 mg Valacyclovir

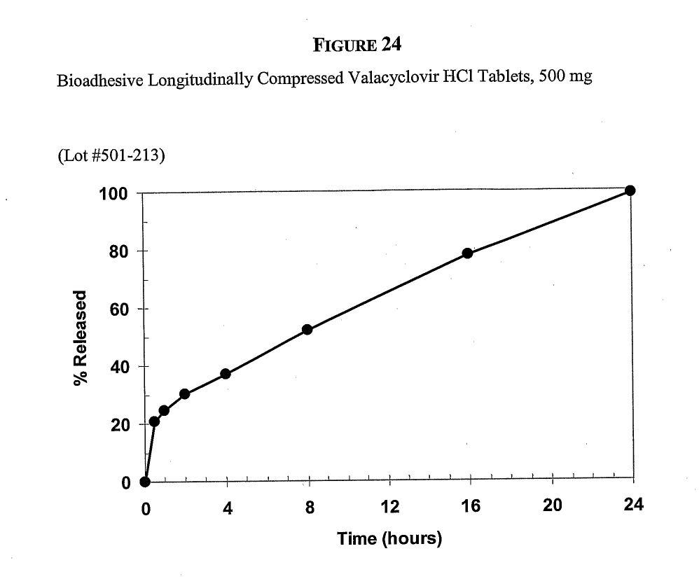

Coated tablets were tested for release profile in 0.1 N HCl at 37±0.5° C., in the USP II dissolution apparatus at 100 rpm. The in vitro dissolution profile of tablets is shown in FIG. 24. As shown in FIG. 24, about 20% of the valacyclovir in the Lot # 501-213 formulation is released in 30 minutes, about 25% in 1 hour, 50% by 8 hours, almost 80% by 16 hours, and all of it is released by 24 hours.

### Example 11

### Longitudinally Compressed Bilayer Tablets containing 2.5 mg Glipizide and 500 mg Metformin HCl (Lot # 502-105)

Longitudinally compressed bilayer tablets were prepared by using a pair of 0.2900″ dies, standard 0.2900″ upper/lower punches, and a manual tablet press described in Example 2. The compound die was filled first with 700 mg of a dry blend of Metformin HCl and excipients (layer 1), and then with 100 mg of a dry blend of Glipizide and excipients (layer 2). The bilayer core tablets were compacted at 4000 psi for 1 second. Each tablet contained 2.5 mg Glipizide and 500 mg Metformin HCl.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 4. The core composition of tablet is provided in Table 11.

TABLE 11
Composition of Glipizide/Metformin HCl, 2.5 mg/500 mg
Core Tablet Formulation

Weight (mg)

| Ingredients | Layer 1 | Layer 2 |
|---|---|---|
| Metformin HCl | 500.0 | — |
| Glipizide | — | 2.5 |
| Hydroxypropyl cellulose | 140.0 | 20.0 |
| Hydroxypropylmethyl cellulose | 35.0 | — |
| Polyethylene oxide | 22.2 | — |
| Compressible Sugar | — | 74.6 |
| Polyvinylpyrrolidone (Crospovidone) | — | 2.5 |
| Magnesium Stearate | 2.8 | 0.4 |
| Total | 700.0 | 100.0 |

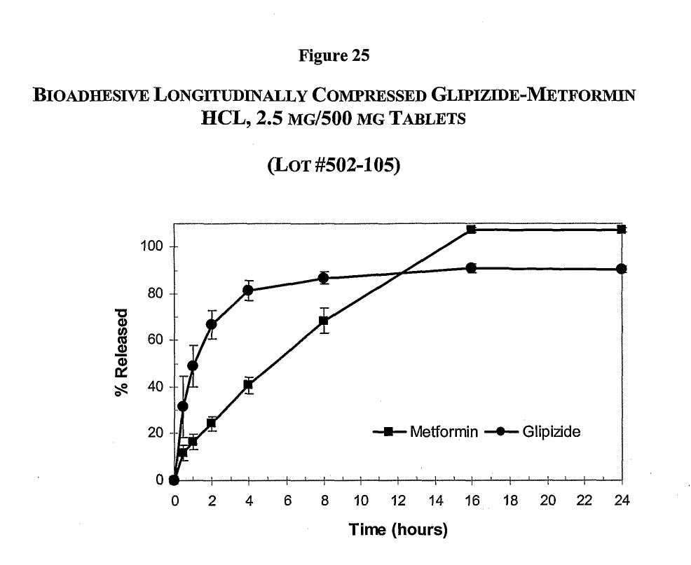

Bioadhesive longitudinally compressed Glipizide-Metformin HCl tablets were tested for release profile in phosphate buffered saline (PBS) solution (pH 6.8) at 37±0.5° C., in the USP II dissolution apparatus at 100 rpm. The in vitro dissolution profile of tablets containing 2.5 mg of Glipizide and 500 mg of Metformin HCl is shown in FIG. 25. As shown in FIG. 25, about 30% of the glipizide in the Lot # 502-105 formulation is released in 30 minutes, almost 70% by 2 hours, and 80% by 4 hours, and plateaus at almost 90% by 8 hours. The release profile for metformin is as follows: a little more than 10% in 30 minutes, about 25% in 2 hours, about 70% in 8 hours, and 100% by 16 hours.

### Example 12

### Longitudinally Compressed Tablets Containing 250 mg Gabapentin and 250 mg Sodium Valproate (Lot #412-010)

Longitudinally compressed core tablets were prepared by using a pair of 0.2900″ dies (Natoli Engineering). The die was filled first with 400 mg of a dry blend of Gabapentin and excipients, and then with 400 mg of a dry blend of Sodium Valproate and excipients. The tablets were prepared by direct compaction at 4000 psi for 1 second using standard 0.2900″ upper/lower punches and the GlobePharma Manual Tablet Compaction Machine (MTCM-1). Each tablet contained 250 mg Gabapentin and 250 mg Sodium Valproate. The composition of core tablets is provided in Table 12.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of these dosage forms is illustrated in FIG. 4.

TABLE 12
Composition of Gabapentin/Sodium Valproate 250 mg/
250 mg Core Tablet Formulation

Weight (mg)

| Ingredients | Gabapentin Layer | Sod. Valproate |
|---|---|---|
| Gabapentin | 250.0 | — |
| Sodium Valproate | — | 250.0 |
| Hydroxypropyl cellulose | 100.0 | 100.0 |
| Polyvinylpyrrolidone | — | 23.6 |
| Hydroxypropylmethyl | 30.0 | 12.0 |
| Polyethylene oxide | 18.4 | — |
| Lactose | — | 12.8 |
| Magnesium Stearate | 1.6 | 1.6 |
| Total | 400.0 | 400.0 |

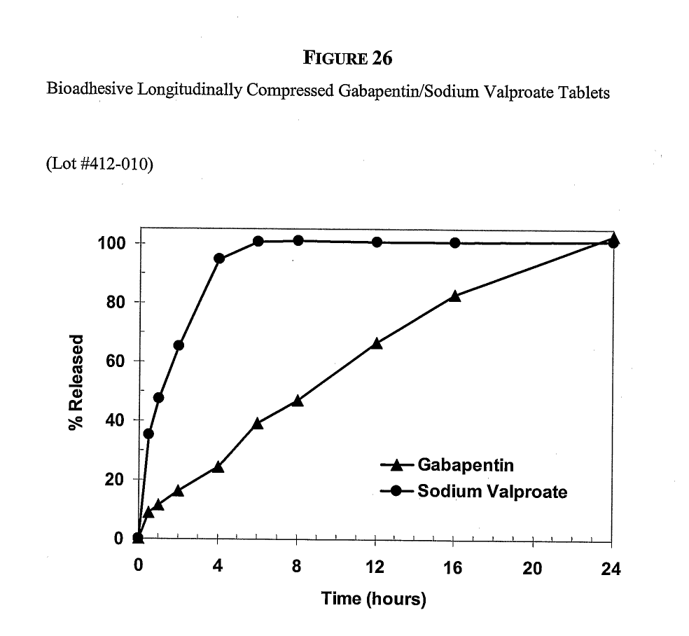

Coated tablets were tested for release profile in phosphate buffered saline (PBS) solution, pH 6.8, at 37±0.5° C., in the USP II dissolution apparatus at 100 rpm. The in vitro release profile of tablets is shown in FIG. 26. As shown in FIG. 26, about 35% of the sodium valproate in the Lot # 412-010 formulation is released in 30 minutes, about 65% by 2 hours, and 100% by 6 hours. The release profile for gabapentin is as follows: almost 10% in 30 minutes, almost 25% in 2 hours, about 45% in 8 hours, a little more than 80% by 16 hours, and 100% by 24 hours.

### Example 13

### Longitudinally Compressed Bilayer Tablets containing 4 mg Rosiglitazone Maleate and 500 mg Metformin HCl (Lot # 503-167)

Longitudinally compressed bilayer tablets were prepared by using a pair of 0.2900″ dies joined together as described previously. The compound die was filled first with 800 mg of a dry blend of Metformin HCl and excipients (layer 1), and then with 100 mg of a dry blend of Rosiglitazone Maleate and excipients (layer 2). The tablets were prepared by direct compaction at 500 psi for 1 second using a standard 0.2900″ lower punch, a special 0.2900″ upper punch (2″ tip length) using a manual tablet press.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 4.

Each tablet contained 4 mg Rosiglitazone Maleate and 500 mg Metformin HCl. The core composition of tablet is provided in Table 13.

TABLE 13
Composition of Metformin HC1/Rosiglitazone Maleate, 4 mg/
500 mg Core Tablet Formulation

Weight (mg)

| Ingredients | Layer 1 | Layer 2 |
|---|---|---|
| Metformin HCl | 500.0 | — |
| Rosiglitazone Maleate | — | 4.0 |
| Hydroxypropyl cellulose | 256.0 | 8.0 |
| Hydroxypropylmethyl cellulose | 40.0 | — |
| Compressible Sugar | — | 85.0 |
| Croscarmellose Sodium | — | 2.5 |
| Magnesium Stearate | 4.0 | 0.5 |
| Total | 800.0 | 100.0 |

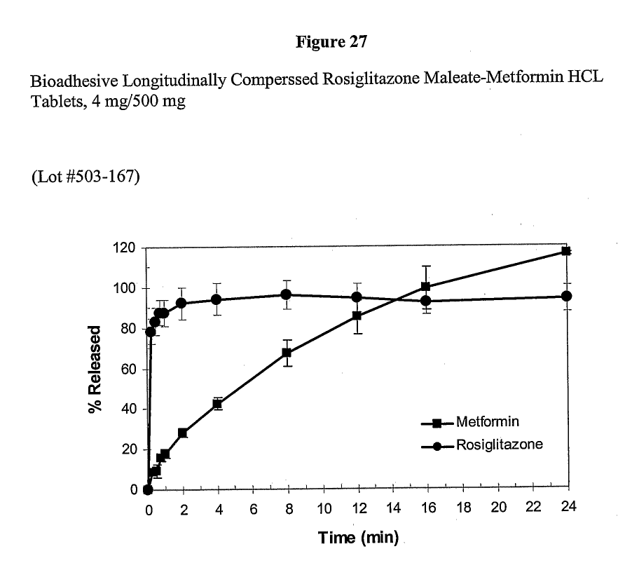

Coated tablets were tested for dissolution in 0.1 N HCl buffer at 37±0.5° C., in the USP II dissolution apparatus at 100 rpm. The in vitro dissolution profiles of tablets are shown in FIG. 27. As shown in FIG. 27, about 90% of the rosiglitazone in the Lot # 503-167 formulation is released in 30 minutes, and it plateaus at about 95% at 2 hours. The release profile for metformin is as follows: almost 20% in 1 hour, about 30% in 2 hours, almost 70% in 8 hours, and 100% by 16 hours.

### Example 14

### Longitudinally Compressed Bilayer Tablets containing 45 mg Pioglitazone HCl and 500 mg Metformin HCl (Lot # 501-237)

Longitudinally compressed bilayer tablets were prepared by using a pair of 0.2900″ dies joined together as described in Example 2. The compound die was filled first with 700 mg of a dry blend of Metformin HCl and excipients (layer 1), and then with 100 mg of a dry blend of Pioglitazone HCl and excipients (layer 2). The tablets were prepared by direct compaction at 500 psi for 1 second using a standard 0.2900″ lower punch, a special 0.2900″ upper punch (2″ tip length), and a manual tablet press described in Example 2.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 4. Each tablet contained 45 mg Pioglitazone HCl and 500 mg Metformin HCl. The core composition of tablet is provided in Table 14:

TABLE 14
Composition of Pioglitazone HC1/Metformin HC1, 45 mg/
500 mg Core Tablet Formulation

Weight (mg)

| Ingredients | Layer 1 | Layer 2 |
|---|---|---|
| Metformin HCl | 500.0 | — |
| Pioglitazone HCl | — | 45.0 |
| Hydroxypropyl cellulose | 140.0 | 15.0 |
| Hydroxypropylmethyl cellulose | 35.0 | — |
| Polyethylene oxide | 22.2 | — |
| Compressible Sugar | — | 37.6 |
| Polyvinylpyrrolidone (Crospovidone) | — | 2.0 |
| Magnesium Stearate | 2.8 | 0.4 |
| Total | 700.0 | 100.0 |

### Example 15

### Bioadhesive Longitudinally Compressed Bilayer Tablets containing 4 mg Rosiglitazone Maleate and 500 mg Metformin HCl (Lot 504-060)

Longitudinally compressed bilayer tablets were prepared by using a pair of 0.2900″ dies (Natoli Engineering). The dies were carefully aligned and firmly joined together to form a compound die. The compound die was filled first with 800 mg of a dry blend of Metformin HCl and excipients (layer 1), and then with 100 mg of a dry blend of Rosiglitazone Maleate and excipients (layer 2). The tablets were prepared by direct compaction at 250 psi for 1 second using a standard 0.2900″ lower punch, a special 0.2900″ upper punch (2″ tip length), and a GlobePharma Manual Tablet Compaction Machine (model MTCM-1).

A 0.4375″ set of die and upper/lower punches was used for press-coating the core tablet. The tablet was placed vertically in the center of 0.4375″ die and 600 mg of a Spheromer™ III polymer blend with ethylcellulose was poured around the tablet inside the die. The core tablet and the polymer were pressed together at 3000 psi for 1 s. The longitudinal-section of this dosage form is illustrated in FIG. 4.

Each tablet contained 4 mg Rosiglitazone Maleate and 500 mg Metformin HCl. The core and coating compositions of tablet are provided in the following tables.

### Core Tablet Formulation

Weight (mg)

| Ingredients | Layer 1 | Layer 2 |
|---|---|---|
| Metformin HCl | 500.0 | — |
| Rosiglitazone Maleate | — | 4.0 |
| Hydroxypropyl cellulose | 276.0 | 4.0 |
| (Klucel EF Pharm) | | |
| Hydroxypropylmethyl cellulose | 20.0 | — |
| (Hypromellose 4K cps) | | |
| Compressible Sugar | — | 89.0 |
| Croscarmellose Sodium | — | 2.5 |
| Mg Stearate | 4.0 | 0.5 |
| Total | 800.0 | 100.0 |

### Coating Formulation

| Ingredients | Weight (mg) |
|---|---|
| Spheromer ™ III, L-Dopa/BMA | 352.0 |
| Ethylcellulose (Ethocel STD 100 FP) | 85.8 |
| Mg Stearate | 2.2 |
| Total | 440.0 |

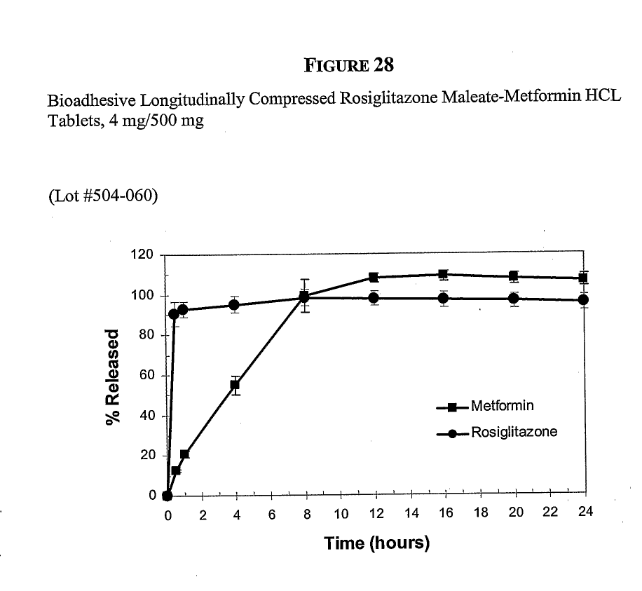

Bioadhesive longitudinally compressed Rosiglitazone Maleate-Metformin HCl tablets were placed in 0.1N HCl at 37±0.5° C., in the USP II dissolution apparatus at 100 rpm. The in vitro dissolution profile of tablets containing 4 mg of Rosiglitazone Maleate and 500 mg of Metformin HCl is shown in FIG. 28. As shown in FIG. 28, about 90% of the rosiglitazone in the Lot # 504-060 formulation is released in 30 minutes, and it plateaus at about 95% at 1 hour. The release profile for metformin is as follows: almost 20% in 1 hour, about 55% in 4 hours, and almost 100% by 8 hours.

### Example 16

### Longitudinally Compressed Trilayer Tablet Formulation containing 30 mg Pioglitazone HCl (Lot # 503-038)

Longitudinally compressed trilayer core tablets were prepared by using a pair of 0.2618″ dies joined together in a similar fashion to the method described in Example 2. The compound die was filled first with 250 mg of a dry blend of Pioglitazone and excipients (layer 1), second with 100 g of compressible sugar (layer 2), and then with 250 mg of a dry blend of Pioglitazone and excipients (layer 3). The tablets were prepared by direct compaction at 4000 psi for 1 second using standard 0.2618″ upper/lower punches and a manual tablet press in a similar fashion to the method described in Example 2. Each tablet contained a total of 30 mg Pioglitazone HCl. The core composition of tablet is provided in Table 16.

The core tablets were coated peripherally on layer 1 and layer 3 with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. Layer 2 was left uncoated. The longitudinal-section of this dosage form is illustrated in FIG. 8.

TABLE 16
Composition of Pioglitazone HC1, 30 mg Core Tablet Formulation

| | Weight (mg) | | |
|---|---|---|---|
| Ingredients | Layer 1 | Layer 2 | Layer 3 |
| Pioglitazone HCl | 15.0 | — | 15.0 |
| Lactose | 162.5 | — | 162.5 |
| Hydroxypropyl cellulose | 62.5 | — | 62.5 |
| Hydroxypropylmethyl cellulose | 8.75 | — | 8.75 |
| Magnesium Stearate | 1.25 | — | 1.25 |
| Compressible sugar | — | 100.0 | — |
| Total | 250.0 | 100.0 | 250.0 |

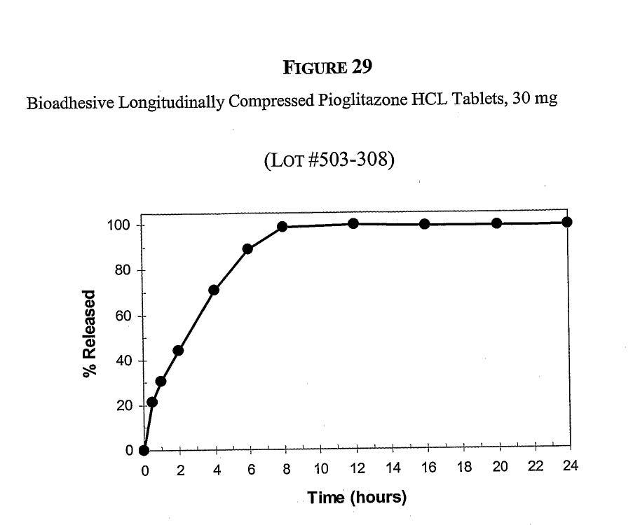

Coated tablets were tested for dissolution in 0.1 N HCl buffer containing 0.3 N KCl at 37±0.5° C., in the USP II dissolution apparatus at 100 rpm. The in vitro dissolution profile of tablets containing 30 mg of Pioglitazone HCl is shown in FIG. 29. As shown in FIG. 29, about 20% of the pioglitazone in the Lot #503-308 formulation is released in 30 minutes, about 45% in 2 hours, almost 70% in 4 hours, about 90% by 6 hours, and 100% by 8 hours.

### Example 17

### Longitudinally Compressed Tablets containing 45 mg Pioglitazone HCl (Lot #502-041, 502-043, 502-045 and 502-047)

Longitudinally compressed tablets were prepared by using a pair of 0.2618″ dies, standard 0.2618″ upper/lower punches, and a manual tablet press in a similar fashion to the method described in Example 2. Each core tablet weighed 750 mg.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 3. Each tablet contained 45 mg Pioglitazone HCl. The core compositions of tablets are provided in Table 17.

TABLE 17
Composition of Pioglitazone HCl 45 mg Core Tablet Formulations

| | Weight (mg) | | | |
|---|---|---|---|---|
| Ingredients | 502-041 | 502-043 | 502-045 | 502-047 |
| Pioglitazone HCl | 45.00 | 45.00 | 45.00 | 45.00 |
| Lactose | 536.25 | 461.25 | 585.00 | 510.00 |
| Hydroxypropyl cellulose | 150.00 | 150.00 | 75.00 | 75.00 |
| Polyvinylpyrrolidone | 15.00 | 15.00 | — | — |
| (Crospovidone) | | | | |
| Croscarmellose sodium | — | — | 41.25 | 41.25 |
| Citric Acid | — | 75.00 | — | 75.00 |
| Mg Stearate | 3.75 | 3.75 | 3.75 | 3.75 |
| Total | 750.00 | 750.00 | 750.00 | 750.00 |

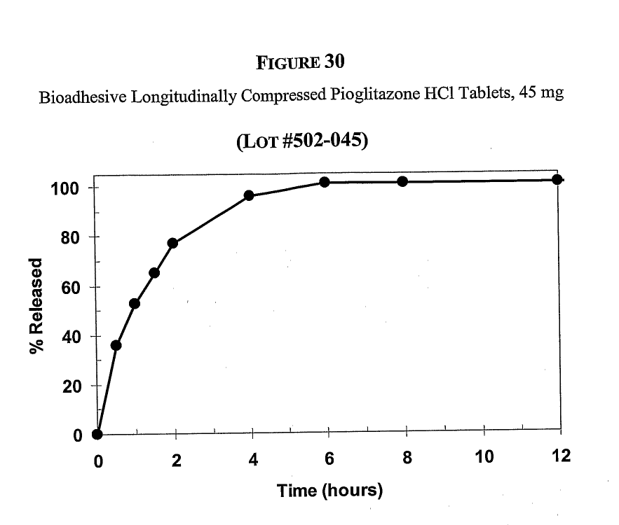

Coated tablets were tested for release profiles in 0.1 N HCl buffer containing 0.3 N KCl at 37±0.5° C., in the USP II dissolution apparatus at 100 rpm. The in vitro dissolution profile of tablets from lot # 502-045 are shown in FIG. 30. As shown in FIG. 30, a little more than 35% of the pioglitazone in the Lot # 502-045 formulation is released in 30 minutes, almost 80% in 2 hours, and nearly 100% by 4 hours.

### Example 18

### Longitudinally Compressed Tablets with Two Pre-compressed Inserts containing 30 mg Piogitazone HCl and 500 mg Metformin HCl (Lot # 503-053)

Longitudinally compressed tablets were prepared by using a pair of 0.2900″ dies (and a 0.2618″ die), standard 0.2900″ upper/lower (and 0.2618″) punches, and a manual tablet press described in Example 2. The 0.2618″ die was filled with 100 mg of a dry blend of Pioglitazone and excipients, and the blend was compacted at 250 psi at 1 s. The 0.2900″ die was filled first with 200 mg of a dry blend of Metformin HCl and excipients. The first pre-compressed Pioglitazone tablet (insert) was placed horizontally on the Metformin layer in the die and 300 mg of Metformin blend was added. The second pre-compressed Pioglitazone tablet was placed horizontally on the top of Metformin layer and another 250 mg of Metformin blend was added. The tablets were compacted at 4000 psi for 1 sec. according to the method described in Example 2.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 10, although the tablet contains two inserts instead of three.

Each tablet contained 30 mg Pioglitazone HCl and 500 mg Metformin HCl. The core composition of tablet is provided in Table 18.

TABLE 18
Composition of Pioglitazone HC1/Metformin HC1, 30 mg/500 mg
Core Tablet Formulation

| | Weight (mg) | | |
|---|---|---|---|
| Ingredients | Matrix | Insert 1 | Insert 2 |
| Metformin HCl | 500.0 | — | — |
| Pioglitazone HCl | — | 15.0 | 15.0 |
| Hydroxypropyl cellulose | 200.0 | 20.0 | 20.0 |
| Hydroxypropylmethyl cellulose | 20.0 | — | — |
| Polyethylene oxide | 76.8 | — | — |
| Compressible sugar | — | 54.6 | 54.6 |
| Croscarmellose sodium | — | 10.0 | 10.0 |
| Magnesium Stearate | 3.2 | 0.4 | 0.4 |
| Total | 800.0 | 100.0 | 100.0 |

### Example 19

### Longitudinally Compressed Tablets with Pre-compressed Insert containing 15 mg Piolgitazone HCl and 250 mg Metformin HCl (Lot # 503-051)

Longitudinally compressed core tablets were prepared by using a 0.2900″ (and 0.2618″) die, standard 0.2900″ (and 0.2618″) upper/lower punches, and a manual tablet press described in Example 2. The 0.2618″ die was filled with 100 mg of a dry blend of Pioglitazone and excipients, and the blend was compacted at 250 psi at 1 s. The 0.2900″ die was filled first with 200 mg of a dry blend of Metformin HCl and excipients. The pre-compressed Pioglitazone tablet (insert) was placed horizontally on the Metformin layer in the die and another 200 mg of Metformin blend was added. The tablets were compacted at 1000 psi for 1 second according to the method described in Example 2.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 10, although the tablet contains two inserts instead of three.

Each tablet contained 15 mg Pioglitazone HCl and 250 mg Metformin HCl. The core composition of tablet is provided in Table 19.

TABLE 19
Composition of Pioglitazone HC1/Metformin HC1, 15 mg/
250 mg Core Tablet Formulation

Weight (mg)

| Ingredients | Matrix | Insert |
|---|---|---|
| Metformin HCl | 250.0 | — |
| Pioglitazone HCl | — | 15.0 |
| Hydroxypropyl cellulose | 100.0 | 20.0 |
| Hydroxypropylmethyl cellulose | 10.0 | — |
| Polyethylene oxide | 38.4 | |
| Compressible sugar | — | 54.6 |
| Croscarmellose sodium | — | 10.0 |
| Mg Stearate | 1.6 | 0.40 |
| Total | 400.0 | 100.0 |

### Example 20

### Longitudinally Compressed Osmotic Tablets Containing 500 mg Valacyclovir (Lot # 505-018)

Longitudinally compressed tablets were prepared by using a special 0.2900″ die, two times longer than ordinary dies (Natoli Engineering). The die was filled with 700 mg of a dry blend of drug and excipients. A special punch, 2″ tip length, was used as the upper punch and to dislodge the tablets from the die. The tablets were prepared by direct compaction at 500 psi for 1 second using the GlobePharma Manual Tablet Compaction Machine (MTCM-1). Each tablet contained 500 mg Valacyclovir. The compositions of core tablets are provided in Table 20.

The longitudinally compressed tablets were first coated completely with a cellulose acetate (CA 398-10) plus PEG 400 based semi-permeable coating. A passageway, 500 μm in size, on the cellulose acetate film was made on each side of the tablet by using a micro-drill.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 11.

TABLE 20
Composition of Valacyclovir HC1, 500 mg Core Tablet Formulation

| Ingredients | Weight (mg) |
|---|---|
| Valacyclovir HCl* | 555.8 |
| Hydroxypropyl cellulose | 141.4 |
| Magnesium Stearate | 2.8 |
| Total | 700.0 |

*Equivalent to 500 mg Valacyclovir

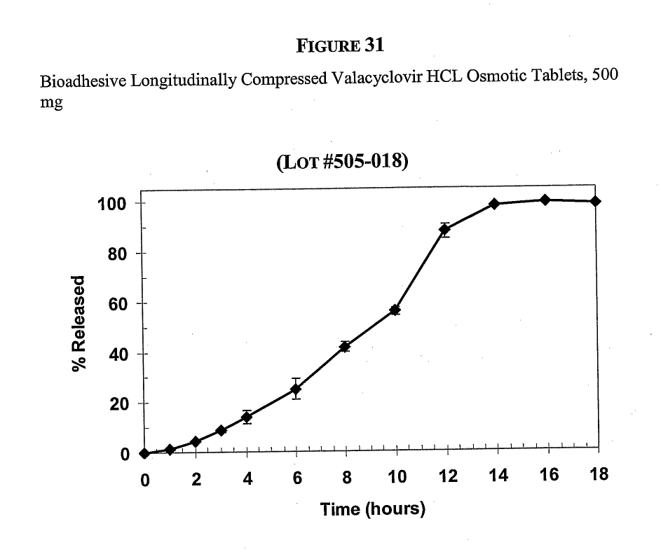

Coated tablets were tested for release profile in 0.1 N HCl at 37±0.5° C., in the USP II dissolution apparatus at 100 rpm. The in vitro dissolution profile of tablets is shown in FIG. 31. As shown in FIG. 31, almost 15% of the valacyclovir in the Lot # 5085-018 formulation is released in 3 hours, about 25% in 6 hours, almost 55% in 10 hours, about 95% by 12 hours, and 100% by 14 hours.

### Example 21

### Longitudinally Compressed Tablets Containing 250 mg Valacyclovir (Lot # 504-027)

Longitudinally compressed core tablets were prepared by using a pair of 0.2900″ dies (Natoli Engineering). The die was filled initially with 100 mg of ethylcellulose composition (Plug II), subsequently with 250 mg of a Valacyclovir immediate release (IR II) dry blend, followed with 100 mg of hydroxypropyl cellulose (Plug I), and finally with 150 mg of a Valacyclovir immediate release (IR I) dry blend. A special punch, 2″ tip length, was used as the upper punch to dislodge the tablets from the die. The tablets were prepared by direct compaction at 500 psi for 1 second using the GlobePharma Manual Tablet Compaction Machine (MTCM-1). Each tablet contained 250 mg Valacyclovir HCl. The compositions of core tablets are provided in Table 21.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer II™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 7.

TABLE 21
Composition of Valacyclovir, 250 mg Core Tablet Formulation

| | Weight (mg) | | | |
|---|---|---|---|---|
| Ingredients | IR I | Plug I | IR II | Plug II |
| Valacyclovir HCl* | 138.9 | — | 138.9 | — |
| Compressible Sugar | 103.85 | — | 103.85 | — |
| Croscarmellose Sodium | 6.25 | — | 6.25 | — |
| Magnesium Stearate | 1.00 | — | 1.00 | — |
| Hydroxypropyl Cellulose | — | 100.0 | — | — |
| Ethylcellulose | — | — | — | 100.0 |
| Total | 250.0 | 100.0 | 250.0 | 100.0 |

*Equivalent to 250 mg Valacyclovir

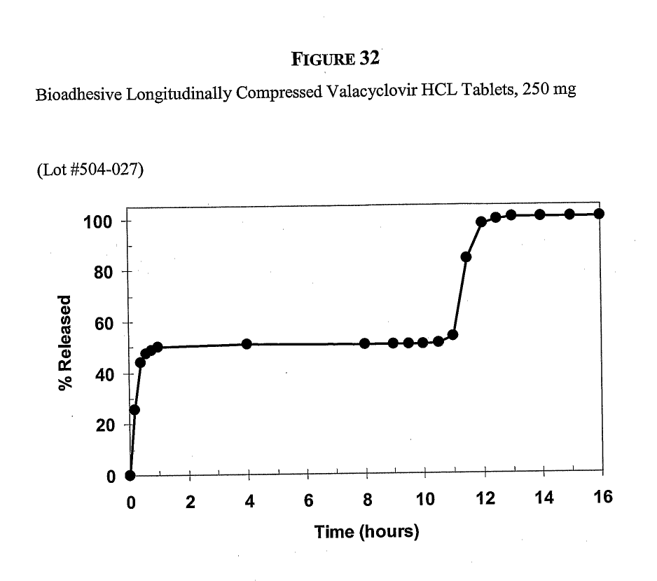

Coated tablets were tested for release profile in 0.1 N HCl at 37±0.5° C., in the USP II dissolution apparatus at 100 rpm. The in vitro dissolution profile of tablets is shown in FIG. 32. As shown in FIG. 32, almost 50% of the valacyclovir in the Lot # 504-027 formulation is released in 30 minutes, and the dissolution profile remains at 50% until 11.5 hours, at which point about 85% of drug has been released. By 12 hours, all the drug has been released.

### Example 22

### Longitudinally Compressed Tablets Containing 250 mg Valacyclovir (Lot # 504-079 and 504-081)

Longitudinally compressed core tablets were prepared by using a special 0.2900″ die. The dies were filled initially with 100 mg of hydroxypropyl cellulose (Plug II), subsequently with 500 mg of a Valacyclovir immediate release (IR) dry blend, and finally with 100 mg of hydroxypropyl cellulose (Plug I). A special punch, 2″ tip length, was used as the upper punch to dislodge the tablets from the die. The tablets were prepared by direct compaction at 500 psi for 1 second using the GlobePharma Manual Tablet Compaction Machine (MTCM-1). Each tablet contained 250 mg Valacyclovir. The compositions of core tablets are provided in Table 22.

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer I (anhydride polymers), Spheromer II (anhydride oligomers blended with pharmaceutical polymers), Spheromer III (catechol-grafted anhydride polymers), or combinations of these polymers can also be applied over the impermeable coating without affecting drug release. The longitudinal-section of this dosage form is illustrated in FIG. 13.

TABLE 22
Composition of Valacyclovir HC1, 250 mg Core Tablet Formulation

| | Weight (mg) | | | | | |
|---|---|---|---|---|---|---|
| | 504-079 | | | 504-081 | | |
| Ingredients | Plug I | IR | Plug II | Plug I | IR | Plug II |
| Valacyclovir HCl* | — | 277.8 | — | — | 277.8 | — |
| Compressible Sugar | — | 207.7 | — | — | 207.7 | — |
| Croscarmellose | — | 12.5 | — | — | 12.5 | — |
| Sodium | | | | | | |
| Magnesium Stearate | — | 2.0 | — | — | 2.0 | — |
| Hydroxypropyl | 100.0 | — | 100.0 | — | — | — |
| Cellulose | | | | | | |
| (Klucel LF Pharm) | | | | | | |
| Hydroxypropyl | — | — | — | 100.0 | — | 100.0 |
| Cellulose | | | | | | |
| (Klucel EF Pharm) | | | | | | |
| Total | 100.0 | 500.0 | 100.0 | 100.0 | 500.0 | 100.0 |

*Equivalent to 250 mg Valacyclovir

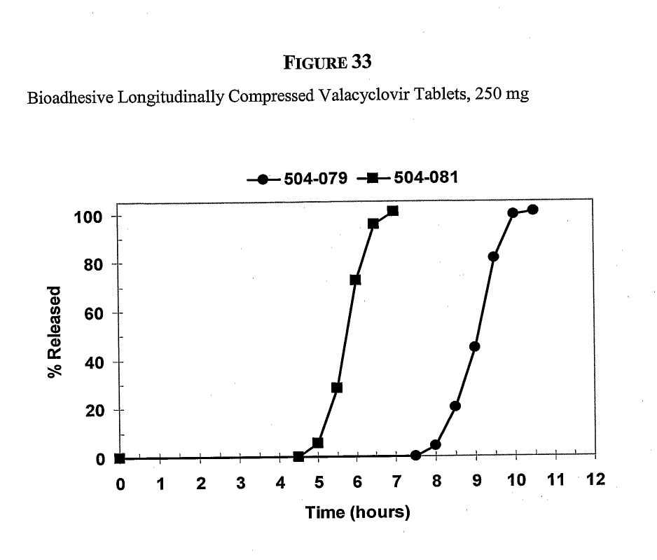

Coated tablets were tested for release profile in 0.1 N HCl at 37±0.5° C., in the USP II dissolution apparatus at 100 rpm. The in vitro release profiles of tablets are shown in FIG. 33. As shown in FIG. 33, no valacyclovir in Lot # 504-081 is released is released until about 5 hours, at which point about 5% of drug is released. Almost 30% of drug is released by 5.5 hours, 75% by 6 hours, and 100% by 7 hours. The valacyclovir in Lot # 504-079 is not released until 8 hours, at which point about 5% of drug is released. Almost 20% is released in 8.5 hours, about 45% in 9 hours, 80% in 9.5 hours, and all the drug by 10 hours.

### Example 23

### Bioadhesive Longitudinally Compressed Tablets Containing 200 mg Levodopa and 50 mg Carbidopa (Lot 507-041)

Longitudinally compressed tablets were prepared by using a special 0.2618″ die, two times longer than ordinary dies (Natoli Engineering). The die was filled with 800 mg of a dry blend of spheronized drug particulates and excipients. Levodopa-Carbidopa particulates ranged from 250 to 425 μm in diameter. A special punch, 2″ tip length, was used as the upper punch and to dislodge the tablets from the die. The tablets were prepared by direct compaction at 2000 psi for 1 second using the GlobePharma Manual Tablet Compaction Machine (MTCM-1).

The LCTs were coated peripherally with a single layer of impermeable PCL film that was heat-sealed to the tablet core. A second film comprising bioadhesive Spheromer II™ (Fumaric Anhydride Oligomer) blended in polycaprolactone was applied over the first film as in Example 2. Optionally, bioadhesive Spheromer™ polymer layers comprising either Spheromer™ I (anhydride polymers), Spheromer™ II (anhydride oligomers blended with pharmaceutical polymers), Spheromer™ III (catechol-grafted anhydride polymers) or else combinations of these polymers layers can also be applied over the impermeable coating without affecting drug release. The longitudinal-section of this dosage form is illustrated in FIG. 15.

Each tablet contained 200 mg Levodopa and 50 mg Carbidopa anhydrous. The composition of particulates and that of core matrix are provided in Table 23a and Table 23b, respectively.

TABLE 23A
Composition of Levodopa-Carbidopa Multiparticulate Formulation

| Ingredients | Wt % |
|---|---|
| Levodopa | 44.0 |
| Carbidopa, Monohydrate | 11.9 |
| Emocel 90M (Microcrystalline cellulose) | 26.0 |
| Ac-Di-Sol (Croscarmellose Sodium) | 6.1 |
| Klucel EF Pharm (HPC) | 5.0 |
| Fast-Flo no. 316 (Lactose, Monohydrate) | 5.0 |
| Citric acid | 1.0 |
| SLS | 1.0 |
| Total | 100.0 |

TABLE 23B
Composition of Levodopa-Carbidopa Spheres in Tablet Formulation

| Ingredients | Wt % | Weight (mg) |
|---|---|---|
| Levodopa-Carbidopa Spheres | 58.25 | 466 |
| Polyox WSR 301 | 41.25 | 330 |
| Mg Stearate | 0.50 | 4 |
| Total | 100.00 | 800.00 |

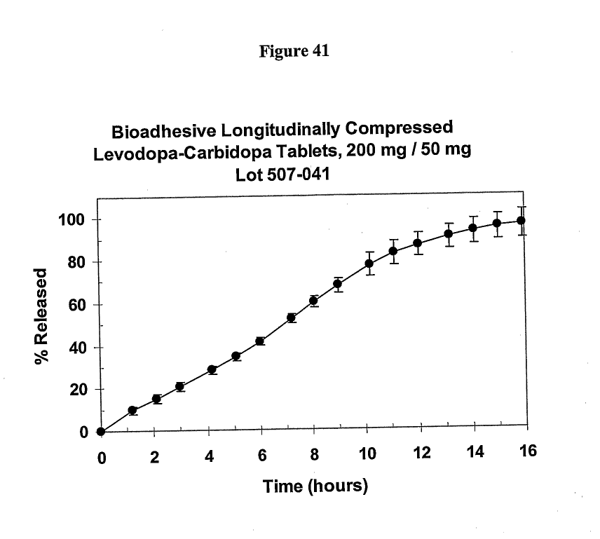

Bioadhesive longitudinally compressed Levodopa-Carbidopa tablets were placed in 0.1N HCl at 37±0.5° C., in the USP II dissolution apparatus at 50 rpm. The in vitro dissolution profile of tablets containing 200 mg Levodopa and 50 mg Carbidopa is shown in FIG. 41. As shown in FIG. 41, about 20% of drug is release in 1 hour, 40% in 4 hours, almost 80% in 10 hours, and almost all of it by 16 hours.

### Example 24

### Bioadhesive Longitudinally Compressed Trilayer Tablets containing 200 mg Levodopa and 50 mg Carbidopa (Lot 506-047)

Longitudinally compressed trilayer tablets were prepared by using a special 0.2618″ die (Natoli Engineering), two times longer than the ordinary dies. The die was filled with three layers of different dry blend formulations of Levodopa, Carbidopa and excipients. The tablets were prepared by direct compaction at 250 psi for 1 second using a standard 0.2618″ lower punch, a special 0.2618″ upper punch (2″ tip length), and a GlobePharma Manual Tablet Compaction Machine (model MTCM-1).

A 0.3287″×0.8937″ capsule shape punch set was used for press-coating the core tablet. A first layer of Spheromer™ III polymer blend with ethylcellulose comprising 200 mg dry powder was added to the die. The pre-compressed tablet was placed horizontally on the polymer bed such that one end of the tablet (layer 1) was positioned in the center in line with the concave edge of the die. A second layer of bioadhesive polymer comprising 240 mg polymer powder was then added to cover the tablet. The core tablet and the polymer layers were then compressed together at 3500 psi for 1 s. The longitudinal-section of this dosage form is illustrated in FIG. 34.

Each tablet contained 200 mg Levodopa and 50 mg Carbidopa anhydrous. The core and coating compositions of tablets are provided in the following tables.

TABLE 24A
Core Tablet Formulation

| | Weight (mg) | | |
|---|---|---|---|
| Ingredients | Layer 1 | Layer 2 | Layer 3 |
| Levodopa | 40.0 | 120.0 | 40.0 |
| Carbidopa, Monohydrate | 10.8 | 32.4 | 10.8 |
| Hypromellose 100 cps (HPMC) | — | 10.0 | — |
| Methocel E5 Prem LV (HPMC) | — | 90.0 | — |
| Glutamic Acid HCl | — | 3.1 | — |
| Corn Starch | — | 3.1 | — |
| Ludipress ® | 98.45 | — | 98.45 |
| Mg Stearate | 0.75 | 1.4 | 0.75 |
| Total | 150.0 | 260.0 | 150.0 |

TABLE 24B
Coating Formulation

| Ingredients | Weight (mg) |
|---|---|
| Spheromer ™ III, L-Dopa/BMA | 352.0 |
| Ethylcellulose (Ethocel STD 100 FP) | 85.8 |
| Mg Stearate | 2.2 |
| Total | 440.0 |

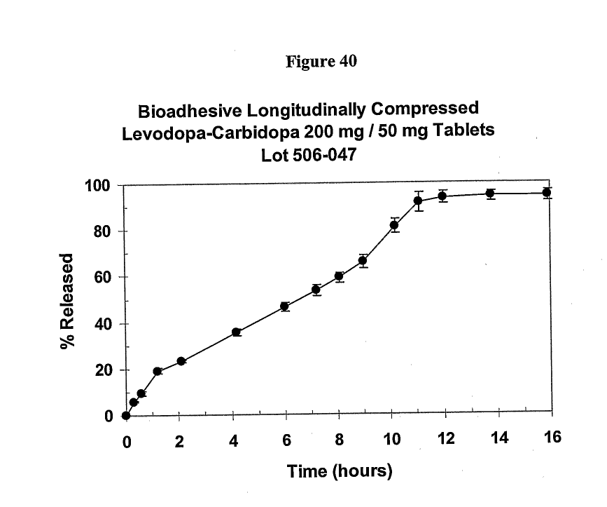

Bioadhesive longitudinally compressed Levodopa-Carbidopa tablets were placed in 0.1N HCl at 37±0.5° C., in the USP II dissolution apparatus at 50 rpm. The in vitro dissolution profile of tablets containing 200 mg Levodopa and 50 mg Carbidopa is shown in FIG. 40. As shown in FIG. 40, about 20% of drug is release in 1 hour, about 35% in 4 hours, almost 60% in 10 hours, 80% in 10 hours, and almost all of it by 12 hours.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A bioadhesive oral dosage formulation, comprising a compressed inner core comprising a therapeutic, prophylactic, or diagnostic agent and an outer bioadhesive polymeric layer disposed over at least a portion of the surface of the core.

2. The formulation of claim 1, wherein the bioadhesive polymeric layer comprises a polymer having one or more catechol moieties.

3. The formulation of claim 2, wherein the catechol moiety is a residue of 3,4-dihydroxyphenylalanine (DOPA).

4. The formulation of claim 1, wherein the bioadhesive polymeric layer comprises a hydrophobic polymer.

5. (canceled)

6. The formulation of claim 1, wherein the bioadhesive polymeric layer comprises a polymer with a hydrophobic backbone.

7. (canceled)

8. The formulation of claim 6, wherein at least 20% of the monomers in the hydrophobic backbone of the polymer contain an aromatic group.

9. The formulation of claim 1, wherein the therapeutic agent is selected from valacyclovir, gabapentin, metformin, pioglitazone, glipizide, sodium valproate, rosiglitazone, levodopa, and carbidopa.

10. (canceled)

11. The formulation of claim 1, wherein the agent is in the form of particles.

12. The formulation of claim 11, wherein the particles are microspheres that release at least 40% of the drug into a fluid of the gastrointestinal tract or into water in less than 30 minutes.

13. The formulation of claim 1, wherein the inner core comprises more than one therapeutic, prophylactic, or diagnostic agent.

14. (canceled)

15. The formulation of claim 1, wherein the inner core comprises one or more absorption enhancers.

16. (canceled)

17. The formulation of claim 1, wherein the inner core is a compressed tablet.

18. The formulation of claim 17, wherein the formulation is an immediate release formulation.

19. The formulation of claim 17, wherein the formulation is an extended release formulation.

20. The formulation of claim 17, wherein the formulation comprises an ascending release formulation.

21. The formulation of claim 17, wherein the formulation comprises a combination of immediate release and extended release formulation portions.

22. The formulation of claim 17, wherein the formulation comprises a combination of extended release formulation portions.

23. A method for preparing a bioadhesive dosage formulation, comprising applying to a compressed inner core comprising a therapeutic, prophylactic, or diagnostic agent, an outer bioadhesive polymeric layer.

24-33. (canceled)

34. A method for administering an agent to a patient, comprising administering to the patient a bioadhesive dosage formulation of claim 1.

35-50. (canceled)

51. A drug-eluting device, comprising a reservoir having a compressed core comprising a therapeutic, diagnostic, or prophylactic agent, an outer bioadhesive polymeric layer disposed over at least a portion of the surface of the core, and one or more areas uncoated by the bioadhesive polymeric layer through which agent from the core can elute from the device.
